SEQUENCE LISTING (101 sequences)

SEQ ID NO: 1 - DNA, 918 bp, Agrobacterium tumefaciens
  source 1..918: mol_type = other DNA
atggcttcacctaaacattttctcgacctgtcggccgttggcccgcaggacctgcggacaattcttgacgatgcgcgcgcgcgcaaggtcgccacgaaggcgggaacggcggaaaagccgctggccggcaagatgctggccatgatcttcgaaaagccgtccacccgcacccgcgtttccttcgatgtgggcatgcgccagctcggcggcgagacccttttcctgtcgggcactgaaatgcagctcggtcgtgcggaaacgatcggcgataccgccaaggtgctgtcgcgttatgtggatgccatcatgatccgtaccacggatcattcgcgcctgctcgagcttgccgaacacgcgacggtgcctgttatcaacggcctgaccgatgatacccatccctgccagatcatggccgacatcatgacgttcgaggaacatcgtggtccggtcaaaggcaagaccattgcctggacgggcgacggcaacaacgtgctgcactctttcgtcgagggctccgcacgtttcggttaccgcatgacgatggcggtgccgatgggttccgaaccgcatgacaagttcatgaactgggcccgcaacaatggcggcgagatcgcgctgtatcatgatgcggacaaggcggtcgcgggtgcggattgtgtcgtcaccgatacctgggtgtccatgaaccaggagcacaaggcgcgcggccacaacatcttccagccctatcaggtcaatgaggccctgatggccaaggcgcagaaggacgcgctgttcatgcactgcctgccggcgcatcgcggtgaagaagtgacggacgccgtgatcgacggcccgcaatcggtggtcttcgatgaggcggagaaccgtctccacgcgcagaaatccgtcatcgcatggtgcatgggcgtgatctga

SEQ ID NO: 2 - DNA, 645 bp, Arabidopsis thaliana
  source 1..645: mol_type = other DNA
atggtgaggggcaaaactcagatgaagagaatagagaatgcaacaagcagacaagtgactttctccaaaagaaggaatggtttgttgaagaaagcctttgagctctcagtgctttgtgatgctgaagtttctcttatcatcttctctcctaaaggcaaactttatgaattcgccagctccaatatgcaagataccatagatcgttatctgaggcatactaaggatcgagtcagcaccaaaccggtttctgaagaaaatatgcagcatttgaaatatgaagcagcaaacatgatgaagaaaattgaacaactcgaagcttctaaacgtaaactcttgggagaaggcataggaacatgctcaatcgaggagctgcaacagattgagcaacagcttgagaaaagtgtcaaatgtattcgagcaagaaagactcaagtgtttaaggaacaaattgagcagctcaagcaaaaggagaaagctctagctgcagaaaacgagaagctctctgaaaagtggggatctcatgaaagcgaagtttggtcaaataagaatcaagaaagtactggaagaggtgatgaagagagtagcccaagttctgaagtagagacgcaattgttcattgggttaccttgttcttcaagaaagtag

SEQ ID NO: 3 - DNA, 3021 bp, Arabidopsis thaliana
  source 1..3021: mol_type = other DNA
atgggcgaacaacaaatctcagttacagtgcctcgtgatagaactgatgaacaggcaatcttggctttgaagaaaggtgcacagcttttgaaatgtcgaagaagaggaaacccaaagttttgtccttttaaactctcaatggatgagaagtacttgatttggtactcaggagaagaggagagacaattgagattgagttctgttataacgattgttcgcggacaaatcacaccaaactttcagaaacaagcacaatctgatcgaaaggaacagtctttttcactcatttatgcaaatggagaacacactcttgatctcatatgtaaggataaagcacaagcagactcatggtttaaaggtcttagagctgtgataacaaagcatcataacattagaaactctgtgaatcatcggagtagtaggggagctcaaagttgcatcaatagtcctgcgggttttatgcgaaggaaacagaatcttggacttcttgaagagactcctgacgtcactcagatacgtagcttatgtggaagtccatcgacgttacttgaagagcggtgtttgtcgaacgggttgtcatgttcttcagatagttttgctgaatctgatgccttgggtccagtttcttcttactatgaaacagattatgatttcaggaactcggattgtgatcgtagtacaggttcagaactttgcaggttttcatcacaaagatttgctgcttctcctcctctttctatcataacacagccggttacaagatccaatgtccttaaggacattatgatatggggagctataacgggtcttatcgatggatcgaagaatcagaacgatgcgttgtctcctaagcttttggaatctgctacgatgtttgatgtacagagtatatctctaggtgcaaaacatgctgcgttggttacaagacaaggtgaagtgttttgttgggggaatggaaatagtgggaagcttggacttaaagttaacatcgatatcgaccatccgaaacgcgttgagtctcttgaggatgttgcagttcgatcagtggcttgtagtgatcatcaaacgtgtgctgttacggaatctggtgagctgtatttatggggaattgatggtggaaccattgaacagtcaggaagccagttcttgactcggaagatatccgatgttcttggcggatctttgactgttttaagtgttgcttgtggcgcgtggcacacggcaattgtgactagttctggtcagcttttcacttatggtagtggaacttttggagttcttggacatgggagtcttgagagtgttacaaagccgaaagaagttgagtctttgagacgcatgaaggtcatatctgtctcttgtggaccgtggcacacggctgctatagtggaaaccgcgaacgaccggaaattctacaacgccaagagttgtggaaagctgtttacttggggagatggcgataaaggacggttaggacatgctgatagcaagagaaagcttgtgccaacttgtgttactgaactcattgatcatgatttcataaaagtctcttgtggatggactttaactgttgctttaagcatttcaggaacggtttatacaatgggaagctcgatccacggacagttaggatgtccgcgggcaaaggataagtcggtaaatgttgttttaggcaatctaacacgacaatttgtcaaggatattgcttcaggttcacatcatgtagctgttctaacttcatttggaaatgtttatacttggggaaaaggcatgaatggacaacttggtttaggtgacgttagagatagaaactctcctgttcttgttgagcctttaggtgataggttggtagaaagtatagcttgcgggttgaatttgacggctgcgatctgtttacacaaagagatctctttgaatgatcaaaccgcttgtagttcttgcaaatcagcttttggattcacaaggagaaagcataactgttacaactgtggtctattgttttgcaatgcatgtagtagcaagaaagcggtaaatgcttcgttagcaccgaataaaagcaagctttctcgggtttgcgattcttgctttgatcatctatggagtataacagagttttcaagaaatgttaagatggataatcacactccgaggatgcagatggttacacgaagagtatctgaggatttgacagaaaaacaatcagagaatgagatgcagaatcttccacaagctaatcgatcttcggatggacagcctcgatggggacaagtctctggtccttctctgtttcgtttcgataagatttcgacaagttcttctttgaatctttcagtctcagcaaggcgaacatcttcaacgaagatttcaacctcgtctgagtcgaataagatcttaaccgaagaaatcgaaaggctaaaagccgtgataaaaaatctccagaggcagtgtgaacttggcaatgagaagatggaagaatgtcaacaagagctcgacaagacatgggaagttgctaaagaagaagcagagaaatcgaaagctgctaaagagataataaaagccttggcatcgaagctccaagcgaataaagagaaaccgagtaatcctctgaagaccggcattgcatgcaatccttctcaagtctcaccaatctttgatgactcaatgtccattccatacctcacaccaatcaccacagctcgttcacaacacgaaaccaaacaacatgtagagaaatgtgtcacgaaatcctcgaaccgtgacagcaacattaagctattagtagatgcatcgccagccatcacaagaacagggtatctccaaaacgaaacacaagactcgtcagcagaacaagttgagcaatacgagcccggtgtatatataactttcacggccttgccttgcggtcagaagacgcttaaacgcgtacggtttagtcggaagaggttttcagagaaggaagcacaaagatggtgggaagagaagcaggttttggtctataacaagtatgatgctgaaatatag

SEQ ID NO: 4 - DNA, 2190 bp, Arabidopsis thaliana
  source 1..2190: mol_type = other DNA
atgcgcatcttctctacattcgtctttcatcgcagacaacaaatcttcaatcttcgtcaattccaaaccaccacaatccttcgcaacccaatctccatcgctccgatccagattccgatggacgccaccgagcagtctctccgtcaatctctttccgagaaatcctcatccgtcgaagctcagggtaacgccgtccgagctctcaaggcttcccgtgcagctaaaccagaaatcgatgctgccattgagcaactcaacaaattgaagctcgagaaatctaccgtcgagaaggagcttcagtctattatcagtagctccggtaatggttcgcttaaccgtgaggcttttcgaaaagctgttgtcaatactctcgagcggcgtttgttctatatcccttcgtttaagatctacagtggcgtcgctggactttttgattatggtcctcctggctgtgctattaaatccaatgttctcagcttttggcgtcaacatttcattcttgaggagaacatgctagaagttgattgtccatgtgtgacaccagaggttgttctcaaggcatctgggcatgtagataagttcactgatcttatggttaaggatgagaaaactggaacatgttaccgtgctgaccacttgctcaaggattattgtaccgagaagctggagaaagatcttaccatctctgctgagaaagctgctgaattgaaggatgttcttgctgttatggaagatttttctcctgaacagctaggtgccaagatcagggagtatgggatcactgctccagacacgaagaatccactctctgatccttacccgtttaatttgatgtttcaaacatccattggcccatctggtttgattcctggttacatgcgtcctgaaactgctcaaggtatttttgtcaactttaaggacttgtactattacaatgggaagaaacttccttttgccgcggctcaaattggtcaagcctttagaaatgagatatctcctcgacaagggcttcttagagttcgtgaattcacgctggcagagattgagcacttcgttgatcctgagaataagtcacatcctaagttctctgacgtagcaaaattggaattccttatgttcccaagggaagaacaaatgtctggccaatctgccaaaaaactttgccttggtgaagctgttgctaaaggtactgtgaacaatgaaactctaggatacttcattgggagagtgtatcttttccttactcgtcttggcattgacaaggaacggctgcgcttccgtcagcatctagcaaatgaaatggcccactatgcagcagattgttgggatgctgaaattgaaagttcatatgggtggattgaatgtgttgggattgcagataggtctgcttacgacttacgtgctcactctgacaaaagtggtactcctcttgtggctgaagagaaatttgcagaacctaaagaagtagagaaacttgtgataactcctgtgaagaaagaactgggtcttgcattcaagggaaatcaaaagaatgtggttgaatctttggaggcgatgaatgaggaagaagctatggagatgaaagcaaccctggaatccaaaggggaagtggagttttacgtgtgtaccctaaagaaaagcgtgaacatcaagaaaaacatggtgtctatatcaaaggagaagaagaaagagcaccagcgggttttcactccatcagtgattgaaccatcgtttgggattggtcggatcatatattgtctgtatgagcattgtttcagcacaaggccaagcaaagcaggggatgagcagttgaacttgttccgtttccctcctcttgtagctcccatcaagtgcacggttttcccgcttgttcagaaccaacaattcgaggaagtagccaaagttatttccaaggaactcgcctctgtcggtatctcccataagattgacatcactggtacatcgatagggaagagatatgcgagaaccgatgagcttggagtgccatttgcaataacagtggactcggatacatcagtgacaatcagagaaagagacagcaaagatcaagtccgagtcaccttgaaggaggcagcttccgttgtgagctcagtctcagaggggaaaatgacgtggcaagacgtctgggcaaccttccctcaccattcttctgctgctgcagacgagtag

SEQ ID NO: 5 - DNA, 1767 bp, Arabidopsis thaliana
  source 1..1767: mol_type = other DNA
atgtcttatgatgagcacacatcatcatcattcacatacataagccaaatgggtgtgtggtggatagtgctggtggtggctgtcttgactcacacggcgtctgccgccgtgagagaatatcattgggaggtggagtacaagtattggtcgccggactgcaaagagggcgccgttatgaccgtcaacggcgagtttcctggtcccaccataaaagccttcgccggagacaccatcgtcgtcaatctcaccaacaaactcaccaccgaaggccttgtcatccattggcatggaatccgtcagttcggaagtccatgggcagatggagcagcaggagttactcaatgcgcaattaaccctggagagacttttacctacaatttcactgttgaaaagccgggaacacatttctaccatggacactatggcatgcagagatcagctgggctatacggatcgttgattgtggacgtggctaaaggaaagagcgagagattgagatacgatggtgagtttaatctcttactcagtgactggtggcatgaggctattccctcccaagaactcggtctttcttccaaacctatgcgctggatcggtgaagctcagagcatattgataaatgggaggggacaattcaattgttcattagcggcgcaatttagcaacaacacatcattaccaatgtgcacgtttaaagaaggtgatcagtgcgccccacagatactccacgtggagccgaacaagacttaccgaatcagactctccagcaccaccgctcttgcctccctcaacttggctgttcagggacacaagctagtggtggtcgaagccgacggcaactacataacgccgttcacgaccgacgatatcgacatatactccggcgaaagctactccgtccttctcaccaccgatcaagacccttcacaaaactattacatctccgtcggcgtccgtggccggaaaccaaacacaactcaggcactcactatattaaattacgtaactgcccctgcttcaaaactcccttcttctcctccaccggtgactccacggtgggacgatttcgaacgaagcaaaaatttctctaagaagatcttctcagcgatgggatcaccatcgccgccgaagaaatacagaaaacggttgattcttctcaacacacagaatctaatcgacggatacacgaaatgggcgatcaacaacgtctctctagtgactccggcaacgccgtatctcggttcggttaaatacaacttgaaactcggatttaaccggaaatcgccgccgaggagttaccgtatggattacgatattatgaatccaccgccgtttcctaacaccactacaggaaacggaatatacgttttcccgtttaatgtaacggtcgacgtgattatccaaaacgctaacgttttaaaaggtattgttagtgagattcatccatggcatcttcacggtcacgatttctgggttttgggttacggtgacgggaaatttaaaccgggaattgatgagaagacgtataatttgaagaatccgccgttacggaatacggcgattttgtatccgtatggatggacggcgataaggtttgtgacggataatccaggggtttggttttttcattgtcacattgaaccgcatttgcatatgggaatgggtgtggttttcgcggagggattaaaccgtattggtaaggtacctgatgaggcgttgggttgtggtttaaccaagcaatttctcatgaaccggaaccgcaattag

SEQ ID NO: 6 - DNA, 1482 bp, Arabidopsis thaliana
  source 1..1482: mol_type = other DNA
atggttttgtctaagacagtttccgaatctgatgtctcaatccattcaacttttgcttctcgttacgtccgcaactctcttccacgattcgaaatgcctgagaactcaatcccaaaagaagcagcttaccaaatcatcaacgacgagctaatgctcgatggtaacccaaggctgaacctagcttccttcgtgaccacatggatggagccagaatgtgacaagctcatgatggagtccatcaacaagaactacgtcgacatggacgagtaccctgtcaccactgagcttcagaaccgatgtgttaacatgatagcacgtctcttcaacgcgccgcttggtgacggtgaagctgccgttggtgttggcaccgtcggatcgtcggaggcgattatgttggccggtttggcttttaagagacaatggcagaataagcgtaaggcccaagggcttccttatgataagcccaatatcgtaaccggtgctaatgtccaggtttgctgggagaaattcgcaaggtatttcgaagtggagcttaaggaagtgaacctaagagaagactattacgtgatggaccctgtaaaggcggtcgaaatggtagacgaaaacacaatttgtgtcgctgccatcctcggttcaacgttaaccggtgaattcgaagacgttaagctcctcaacgacctccttgtcgagaaaaacaagcaaaccggatgggacacgccaatacacgtggacgcagcgagtggtgggtttattgctccgttcttgtatccggagctggagtgggatttccggctaccgttggttaagagtattaatgtgagtggtcacaaatacggtttggtttacgccggtattggttgggttgtatggagaaccaaaaccgatttgcctgatgaacttatcttccatatcaattatcttggcgctgatcaaccaacctttacactcaacttctccaaaggttcaagtcaagtgattgctcagtactaccagctgattcgtcttggattcgagggttatcgcaatgtgatggataattgtcgggaaaacatgatggtactaagacaaggattagagaaaacgggacgttttaaaatcgtctccaaagaaaacggtgttccgttagtggcgttttctctcaaagatagtagccgccacaacgagttcgaggtggcccatacactccgtcgcttcggctggatcgttccggcctacacgatgcctgcggatgcgcagcatgtcactgtccttcgagttgttatccgagaagatttctctcgaaccttagccgagagattggtagctgatttcgagaaggttctacacgagctcgatacgcttccggcgagggttcacgccaagatggctaatggaaaagttaacggtgttaagaagacgccagaggagacgcagagagaagtcacggcctactggaagaagttgttggagactaagaagaccaacaagaacacaatttgctaa

SEQ ID NO: 7 - DNA, 849 bp, Arabidopsis thaliana
  source 1..849: mol_type = other DNA
atgggtactagagctcagcagattcctttacttgaaggtgagactgataattacgatggtgttactgtaaccatggtggaacctatggattctgaggtttttactgaaagtcttagggcttctctttcgcattggagagaagaggggaagaagggaatttggataaagctgcctcttggattggctaatcttgtggaggctgcagttagtgaaggatttagatatcaccacgcggagcctgagtacttgatgcttgtatcttggatctctgaaactcctgatacaatcccagccaatgcttctcatgttgtaggtgctggtgctttggtcatcaacaaaaatactaaagaggtcctcgttgtccaggagaggagtgggtttttcaaagataaaaatgtgtggaagctgcctactggtgttatcaacgagggcgaggatatatggactggagtagctagggaagtggaagaagaaactggaattattgcagattttgtcgaagtactggctttcaggcaaagccacaaagccatcttaaaaaagaaaacagatatgtttttcctgtgtgtcttaagtccgcgctcttacgatattactgaacaaaaatctgagatcttgcaagctaagtggatgccgatccaagagtatgtagaccaaccatggaacaagaagaacgagatgttcaagttcatggctaacatttgccaaaagaagtgtgaggaagaatacttgggattcgccattgtgccaactaccacatcatctggtaaagagagctttatctactgcaatgcggatcatgccaagcgccttaaagtatcgcgtgaccaagcctctgcttctctctga

SEQ ID NO: 8 - DNA, 1383 bp, Arabidopsis thaliana
  source 1..1383: mol_type = other DNA
atgacagttgttggagatgttgctccaattccaaggaggaacagttctacttgctctaatgatatcgctgctccgttattaccagagtgtcatggagatgaagttgctcatgatgaattcaatggagcttcttttagtggcgcggtttttaacctcgccacgactataattggtgctggtatcatggctttgcctgctacaatgaagattcttggacttggacttggaatcacgatgattgttgttatggctttcttgactgatgcgtcgattgagttcttgcttaggtttagtaaagctgggaagaatcgatcttacggtggtttaatgggtggttcttttggtaatcctggaaggattttgcttcaagtcgcggttttagttaataacatcggtgttttgattgtatacatgatcattataggtgatgtgttggctggaaagacggaagatggtatccatcatttcggtgttctcgaaggatggtttggtcaccattggtggaatggaagagctgctattcttctgattactactcttggtgtgtttgctccattggcttgcttcaagcgaatcgattctttgaaatttacatcggccttatccgtggctctggcggttgtgtttctcattattactgcgggaatttcaattatgaagttgatcagtggcggtgtggcgatgccaagattgttaccagatgttactgacttaacatctttctggaatctcttcacagtcgtacctgttcttgtcacagcattcatttgccattacaatgttcacagtatacagaacgagctcgaagacccctctcagataagacctgttgttcgatcagcacttatgctctgctcatctgtttacataatgacaagtattttcgggttcctcttgtttggtgatgatactcttgatgatgtccttgcaaactttgacactgatcttggaatcccttttggttctatccttaatgatgcggttcgagttagctacgcgcttcatttgatgctcgtgttcccgattgttttctaccctcttcggattaacattgacgggctcttgttcccttccgctcgatcattatctacctcaaatgtgaggttcggttgcctcactgccggtctcatctctgtaatcttcttgggtgcaaacttcatcccaagcatttgggatgctttccaattcactggagcaaccgccgctgtttgtctcggcttcatcttcccagcttctatcatactaaaggatcgtcatgacaaagcaacaaacagggacacaaccttagctattttcatgattgttcttgcggtattgtccaatgcaatcgccatttacagcgatgcttatgcgctattcaagaagaacgctcctcgtgagtaa

SEQ ID NO: 9 - DNA, 402 bp, synthetic construct
  source 1..402: mol_type = other DNA; note = Arabidopsis thaliana putative AP2/EREBP transcription factor; with A33V mutation
atggatcaaggaggtcgtagcagtggtagtggaggaggaggagccgagcaagggaagtaccgtggagtaaggagacgaccttggggtaaatacgccgtggaaataagagattcgaggaagcacggagagcgtgtgtggctagggacattcgacactgcggaagacgcggctcgagcctatgaccgagccgcctattcaatgagaggcaaagctgccattctcaacttccctcacgagtataacatgggaaccggatcctcatccactgcggctaattcttcttcctcgtcgcagcaagtttttgagtttgagtacttggacgatagcgttttggatgaacttcttgaatatggagagaactataacaagactcataatatcaacatgggcaagaggcaataa

SEQ ID NO: 10 - DNA, 1074 bp, Arabidopsis thaliana
  source 1..1074: mol_type = other DNA
atggtaatatatcatcgcaaagtagtctttacgtacgtaagagcaaagcgtttctatcatttccttaatattgaaatggtgacagacttcaaatccctactaccagttatcgacatcagtcctttactggccaaatgtgatgatttcgacatggctgaggatgcgggggttgtggaagtagtcggaaaactggacagggcatgtcgggacgtcggattcttctacgtgattggtcatggaatatcggatgatcttataaataaggtgaaagagatgacgcatcagttcttcgagcttccttacgaggagaaacttaagatcaagattactccaactgctggatacagaggataccaaagaattggagtaaattttacgagtgggaaacaagatatgcacgaagccattgattgttacagagagttcaagcaagggaaacatggggacatcggaaaggtcttggaagggccaaaccagtggccaggaaatcctcaagagtacaaagatttaatggagaagtatattaagctgtgcacagatctttctagaaatatcttaaggggaatctcattagcccttggtggatcaccttatgagtttgagggaaagatgcttagagaccctttttgggtaatgcgtatcattggttatccaggtgtaaaccaagaaaatgttattggatgtggagctcacactgactatggcttgttgacgcttataaatcaagatgatgataaaactgctcttcaggtgaaaaacgtggacggtgattggataccagctattccgatccctggatcatttatctgcaacatcggtgacatgttaacgatactatcaaacggagtttaccaatccacactacataaagtaatcaataactctcctaaataccgtgtatgtgttgcatttttctacgagactaacttcgaggcggaggtagagccacttgatatctttaaagagaagcatccacgaaaagaaacatctcaagttgccaaaagagtcgtctatggacagcatctgattaacaaagtcctgacaacctttgcaaatttagtggaaaacagttaa

SEQ ID NO: 11 - DNA, 1116 bp, Arabidopsis thaliana
  source 1..1116: mol_type = other DNA
atgcctgtacttttctcctctcgttctctcatactctccattattgtccccttacttatatctatcgctctctacaaactcgacacattcgaccccgctattgttccttccgatgcattcacatcctctgctacttcactcccgccgcttataaacgacgaatttctcaccggagctgagtttatcggtgtcggtcttctcaatatacccgaagatatcgcctaccataaggaatctaatctcatctacactggttgtgttgatggatgggtgaaacgagtcaaggtcgccgactcggttaatgactcagtcgttgaggattgggtcaatactggtggtagaccgctcggaatcgcgttcggaatccacggcgaagtcattgtcgcagacgtacacaagggactgttgaatataagcggtgacggaaagaagacggaattgttgacggacgaagcggacggtgtgaagtttaagctgacggatgcagttaccgttgcagataacggcgttttgtatttcacagatgcttcttacaagtacactctcaatcaattgagtttagacatgttagaagggaaaccttttggacgactcttgagctttgatcccaccacacgtgttacaaaagtacttctcaaagacctctacttcgctaatggcatcaccatctctcctgatcaaactcatctcatcttctgtgaaactccaatgaaaaggtgcagcaagtattacatcagtgaggagcgtgtggaggtattcactcaaagcttaccaggttatccagataacattcgttacgatggagatggacattactggattgcattgccttcgggagtcacgacattgtggaatatctcgttgaaataccctttcttgaggaaactcacagcaatggtggctaagtacggcgtggatcttatgtttatggagaatgcaggcgttttacaggtggatttggatggaaatcccatcgcgtactaccatgatccaaaactctctcacatagccacatgtgacaagattgggaaatatctctattgtggaagtctctcgcaatcacatatcctccgacttgatctcctgaaatatcctgcacagaacaagaaactttga

SEQ ID NO: 12 - DNA, 1407 bp, Arabidopsis thaliana
  source 1..1407: mol_type = other DNA
atgggtcgtgtccgaaaatcagatttcggttccattgttcttgttctgtgttgtgtcctaaatagcttactctgtaatggaggcatcaccagtagatatgtcagaaaattagaagcaaccgttgatatgcctcttgatagcgatgtttttcgtgttccttgtggctacaatgctcctcaacaggtgcatataacacaaggagatgtagaaggaaaggcagtgatagtatcatgggtgacacaagaagcaaaaggatctaacaaagtcatttactggaaagagaatagcaccaaaaagcacaaagctcatggcaaaaccaatacttacaagttctacaattatacttctggttttatccatcattgccctatcagaaacttagagtatgacaccaagtactattatgtgttaggtgtgggacaaacggagcgtaagttttggttcttcactcctcctgagatcggtcctgatgttccctacacttttggtctcattggggatcttgggcagagttatgactcaaacataaccttaacacattatgagaataacccaacaaaagggcaagcggttttgttcgtcggcgatatctcatacgctgatacttatccggatcatgacaataggagatgggacagttggggaagatttgctgaaagaagcacagcttatcagccttggatttggactaccggaaaccatgaactcgattttgccccggagattggtgaaaacagaccgtttaagccgttcacgcataggtaccgaactccttaccgatcatcaggcagcaccgaaccattctggtactcgataaagagaggaccggcttacataatcgtgctagcttcatattcagcatatggaaaatacacaccacagtaccaatggctcgaagaggagttcccaaaggttaacagaacggaaactccgtggttgattgttctgatgcattcaccatggtacaacagctatgattaccattacatggaaggtgaaacaatgagggtaatgtatgaggcttggtttgtcaagtacaaagtagatgtagtttttgcaggtcatgtccacgcctacgaaagatcggaacgtgtatcaaacatagcttacaatgttgttaacggcatttgcactccagtcaaagatcaatcagctccagtctatatcaccattggtgatggaggcaatattgagggcttggctaccaaaatgactgagcctcagcctaagtactctgcctttagagaagcaagcttcgggcatgctatattctcgataaagaacaggacgcatgctcactatggatggcataggaaccatgatggttatgctgtggagggtgacagaatgtggttttataacagattttggcatcctgttgatgattctccttcttgtaattcttga

SEQ ID NO: 13 - DNA, 1500 bp, Arabidopsis thaliana
  source 1..1500: mol_type = other DNA
atgttattgaaacccggaaataagcttgtctcgccggagacgagtcatcatcgtgactctgcctcaaactcatcaaaccataaatgtcaacaacaaaaaccgcgtaaagataagcagaagcaagttgagcaaaacacaaagaagattgaagaacatcaaataaaatcagagtcgactctgttaatcagcaatcacaacgttaacatgagctcccaaagcaacaacagcgaaagcacatccactaataattcctctaagcctcacacgggaggagatattaggtgggacgcagtgaattccttgaaatctagaggaatcaagcttggaatcagcgattttcgtgttttgaaacggctaggctacggagatatcggtagtgtttatcttgttgaactcaaaggggcaaatcccacgacgtatttcgcgatgaaagtgatggacaaggcttctcttgtgagcaggaacaagctgttgagagctcagactgaaagagagatcttgtctcagcttgatcatcctttcttgcctactctttactctcacttcgaaactgataaattctattgcttggtgatggagttttgtagtggtggaaatctctattccttgagacagaagcaacccaacaagtgtttcacagaagacgctgcaaggttctttgcatcagaagtgttattagcattagagtacttacatatgctcggaatcgtataccgagatttaaagccagagaacgttctggttcgagacgacggtcacattatgctctccgatttcgatttatccctccgatgttcagtcaacccaacacttgtcaaatctttcaacggcggtggaaccaccggaatcattgacgacaatgcggcggtacaaggatgttaccaaccatccgcattcttcccacgaatgctccaatcctcgaagaaaaaccgtaaatccaaatccgatttcgatggatcgttaccagaactcatggcagagccgacaaacgtgaaatcaatgtcttttgtcggaacacacgagtatttagcgccggagattatcaaaaacgaaggtcacggaagcgccgtcgattggtggacgttcgggatattcatctacgagcttcttcatggagcaacaccatttaaaggacaaggaaacaaagccacgctttataatgtaatcggacaacctcttagattcccggaatattctcaggttagctcgacggcgaaggatttgatcaaaggtttattggtaaaagagccgcaaaacagaattgcgtataagcgaggcgcgacggagatcaagcaacatccattttttgaaggtgtgaattgggcgttgattcgtggagaaacaccgccacatttgccggagccggtggatttctcgtgttatgtgaagaaggagaaggagtctttgccgccggcggcaacggaaaagaagagcaagatgtttgatgaagctaataagagcggtagtgatcctgattacattgtttttgaatatttttag

SEQ ID NO: 14 - DNA, 4659 bp, Chlamydomonas reinhardtii
  source 1..4659: mol_type = other DNA
atggaggcggtcgcgaccgttgtggccaacgagcccaaggttgctgatatggaggagattctcgccgagcgcgatgcctgcggcgtgggcttcatcgcgaacctgaagaacgttcagagccacaccgtcgtgaagcaggctctgaccgctctgggctgcatggagcaccgcggtgcttgctctgcggacgatgactcgggagatggtgcgggtcttatgactcaaatcccctggaagctgctgaagaaggagatgcccgcgctgaacgagacgaccacgggtgtcggcatggtgttcatgcccaatgatgacgctctagaggcgcagtgcaagcagatcctggagcaggtctgcgccaaggagggcgtgaaggtggtgggctggcgcaaggtgcccgtcaaccacgacatcgtgggccgcttcgccaaagtgacggagccgcgcatctggcaggtcctgattgagggcaagtccggtcaggtcggcgacgagctggagcgtgagctgttcctggtgcgcaagctggtggagaaggctaagaacgccgcgctgcccgctgagttcgctcccgacttctacatttgcacgctgtccagccgcaccattgtgtacaagggcatgctgcgctcggcggtcgtgggcaccttcttccgcgacctggaaaaccccgacttcgagtcggcgttcgccatctaccaccgccgcttctccaccaacaccacccccaagtggccactggcgcagcccatgcgcgtgctgggccacaacggcgagatcaacaccctgcagggcaacctgaactgggttgcctcgcgcgagcacgagctgagcaaccccatctggaagggccgcgaggcggagctgacgccgctgtgcaacgccgcgcagtcggacagcgccaacctggacaacgtggcggagctgctggtgcgcaccggcaccgacccgcaggacgcgctcatgctgctggtgccggaggcctaccgcaaccaccccgacctcatgaaggagtaccccgaggtggtggacttctacgagttctacgagggcctgcaggagggctgggacggccccgcgctgctggtgttctccgacggcaagcgcgtgggcgcccgcctggaccgcaacggcctgcgccccgcgcgcttctggcagaccaaggacgacatgatttacgtggcctccgaggtgggcgtgctgggtgacgccatcaccaacgccgagaacatcgtggccaagggccgcctgggccccggccagatggtgtgcgccgatctggagaagggcattttctcggagacgtcggccatcagcaagctggtggctggccgcaagccctacaaggagtggctggccgcctcgctgcgccgcctgacggacctgggcgagagcaccttcctgaacgagcccatgtacgacgccgctaccatgctgcgcctgcagtccgccatcggcatggacgccgagaacgcgcagatggtggtggagagccaggcgcagaccggcgtggagcccacctactgcatgggtgacgacatcccgctggccgtgctgtcggacaagccgcacatgctgtacgactacttcaagcagcgcttcgcgcaggtgaccaacccgcccatcgacccgctgcgtgagggcctggtcatgagcctggagatgcgcctgggcgcgcgcggcaacctgctgaaccccggcgccgacagctacaagcaggtgctactggactcgcccatcctgctggagagcgagatgcaggccatctccaccgacaaggtgctgggctccaagaccttcaagctgttcttcgaggccggcaagcccggcgccatggaggcggcgctgaagaagctgtgctcggacgtggaggccgccgtcaaggccggctgccagtgcgtggtgctgtcggaccgccccgacggcggtatggacgccggtaaggcgcccatccccgcgctgctggccaccggcgccgtgcaccaccacctgatccgcacctcgctgcgcagcgacacctcgatcgtggtggacaccgccacctgctacagcacccaccacgccgccatgctgatcggcttcggcgcgcacgccatctgcccctacctgggctacgagaccagccgccagtggcgcctgtcggcgcgcacgcagagcctgatcaaggccggcaaggtgccggacatcagcgtcaaggtggcgcagaagaacttcaagaagtcgctggagaagggcgtgctcaagatcctgtccaagatgggcatctcgctgctgtcgtgctaccacggcgcgcagatcttcgaggcgtacggcctgggcaaggacgtgatggacatgtgcttcaagggcaccgtgtcgcgcattggcggcatgagcctggcggacctgcagcgcgagtccgagagcctgtgggccaagggcttccccgagaaggccatgaccaagctggaggactacggcttcatccagtccaagcccaagggtgagttccactccaacaaccagaccatggccaagctgctgcacaaggccatcggcctgggcaacggctccgctgccgacaaggacgcctacaaggcctaccagcagcacttcgcggactcgcccgtggccgtcctgcgcgactgcctggagttcaagtcggaccgcggccccatctccattgaccaggtggagcccgccgccgccatcatggagcgcttctgcaccggtggcatgtcgctgggcgccatctcgcgcgagacgcacgagaccatcgccatcgccatgaaccgcatcggcggcaagtccaactcgggcgagggcggcgaggacccgatccgctggctgcacctgtcggacgtggacggcgagggcaagtccgccaccgcctcctacctgcgcggcctgcgcaacggcgacaccgccacctccaagatcaagcaggtggcgtcgggccgcttcggcgtcacgcccgagtacatcatgaacgcggagcagatggagattaagattgcgcagggcgccaagcccggtgagggcggccagctgcccggccagaaggtgtcgccctacatcgcgcagctgcgccgctccaagcccggcgtgccgctcatctcgccccctccccaccacgacatctactccattgaggacctggcgcagctcatctacgacctgcaccaggtcaacccccgcgccaaggtgtcggtcaagctggtggccgaggccggcatcggcgtcgtcgccagcggcgtggccaaggccaacgccgacatcatccaggtgtctggccacgacggcggcaccggcgcctcgcccatctcgtccatcaagcacgcgggcggccccatggagatgggcctggcggagacgcaccagacgctggtgcgcaacgagctgcgcgagcgcgtggtgctgcgcgtggacggcggcgtgcgcaacggccgcgacgtgctgatgggcgcgctgatgggcgccgacgagttcggcttcggcaccgtggccatgattgccaccggctgcatcatggcgcgcgtgtgccacaccaacaactgccccgtgggcgtggcgtcgcagcgcgaggagctgcgcgcgcgcttccctggcgcccccgaggacctggtcaactacttccacttcgttgccgaggaggtccgtgccgagctggccaacatgggctaccgcagcctggatgaggtcattggccgcgccgacctgctgaagcagcgctccgtcaagctggccaagaccgagggcctggacctgtccttcctcaccaccttcgccggcgccagcggcaagtcctcgacccgccgcgcgcaggaggtgcacgacaacggcccgcagctggacgaccgcatcctggcggaacccgaggtgatggccgccatcaaggaccacaagaccgtgtccaaggccttcgagatcgtcaacgtggaccgctcctcgctaggccgcgttgccggtgtcatcgccaagcaccacggcgacagcggcttccagggcaaggtcaagctgacgctgaccggctcgggcggccagtcgttcggctgcttctgcgtcaagggcctggaggtgaagctggtgggtgaggccaacgactacgtgggcaagggcatgaacggcggcgagatcgccatcgtgccgcccgccaactcgcccttcaagcccgaggaggcctcgctggtcggcaacacttgcctgtacggcgccaccggcggccggctgttcgtgaacggccgcgccggcgagcgcttcgcggtgcgcaactcgctagcggaggcggtggtggagggcgcgggcgaccactgctgcgagtacatgactggcggctgcgtgatcgtgctgggctcggtgggccgcaacgtggcggcgggcatgacgggcggcctgggctacttcctggatgaggacggctcgttcacggacaaggtcaacacggagatcgttagcgtgcagcgcgtgatcaccaaggccggagaggcgcagctgcgggggctgctggaggcgcacgtggcgcacactggcagcgccaaggccaagagcctgctggccaactgggaggcgtcgctgggcaagttctggcagctggtgccgcccgcggagaagaacaccgccgaggtcaacccctcggtggcgcagccggcggcggctggcgccaaggtggctgtcagcgcatga

SEQ ID NO: 15 - DNA, 1350 bp, synthetic construct
  source 1..1350: mol_type = other DNA; note = Chlorella sorokiniana NADP-specific glutamate dehydrogenase (NADP-GDH), N terminus residues 1 to 74 truncated
atgcacggcatcaagaaccccgagctgcgccagctgctgaccgagatcttcatgaaggacccggagcagcaggagttcatgcaggcggtgcgcgaggtggccgtctccctgcagcccgtgttcgagaagcgccccgagctgctgcccatcttcaagcagatcgttgagcctgagcgcgtgatcaccttccgcgtgtcctggctggacgacgccggcaacctgcaggtcaaccgcggcttccgcgtgcagtactcgtccgccatcggcccctacaagggcggcctgcgcttccacccctccgtgaacctgtccatcatgaagttccttgcctttgagcagatcttcaagaacagcctgaccaccctgcccatgggcggcggcaagggcggctccgacttcgaccccaagggcaagagcgacgcggaggtgatgcgcttctgccagtccttcatgaccgagctgcagcgccacatcagctacgtgcaggacgtgcccgccggcgacatcggcgtgggcgcgcgcgagattggctaccttttcggccagtacaagcgcatcaccaagaactacaccggcgtgctgaccggcaagggccaggagtatggcggctccgagatccgccccgaggccaccggctacggcgccgtgctgtttgtggagaacgtgctgaaggacaagggcgagagcctcaagggcaagcgctgcctggtgtctggcgcgggcaacgtggcccagtactgcgcggagctgctgctggagaagggcgccatcgtgctgtcgctgtccgactcccagggctacgtgtacgagcccaacggcttcacgcgcgagcagctgcaggcggtgcaggacatgaagaagaagaacaacagcgcccgcatctccgagtacaagagcgacaccgccgtgtatgtgggcgaccgccgcaagccttgggagctggactgccaggtggacatcgccttcccctgcgccacccagaacgagatcgatgagcacgacgccgagctgctgatcaagcacggctgccagtacgtggtggagggcgccaacatgccctccaccaacgaggccatccacaagtacaacaaggccggcatcatctactgccccggcaaggcggccaacgccggcggcgtggcggtcagcggcctggagatgacccagaaccgcatgagcctgaactggactcgcgaggaggttcgcgacaagctggagcgcatcatgaaggacatctacgactccgccatgggcgcgtcccgcgagtacaatgttgacctggctgcgggcgccaacatcgcgggcttcaccaaggtggctgatgccgtcaaggcccagggcgctgtttag

SEQ ID NO: 16 - DNA, 1344 bp, Escherichia coli
  source 1..1344: mol_type = other DNA
atggatcagacatattctctggagtcattcctcaaccatgtccaaaagcgcgacccgaatcaaaccgagttcgcgcaagccgttcgtgaagtaatgaccacactctggccttttcttgaacaaaatccaaaatatcgccagatgtcattactggagcgtctggttgaaccggagcgcgtgatccagtttcgcgtggtatgggttgatgatcgcaaccagatacaggtcaaccgtgcatggcgtgtgcagttcagctctgccatcggcccgtacaaaggcggtatgcgcttccatccgtcagttaacctttccattctcaaattcctcggctttgaacaaaccttcaaaaatgccctgactactctgccgatgggcggtggtaaaggcggcagcgatttcgatccgaaaggaaaaagcgaaggtgaagtgatgcgtttttgccaggcgctgatgactgaactgtatcgccacctgggcgcggataccgacgttccggcaggtgatatcggggttggtggtcgtgaagtcggctttatggcggggatgatgaaaaagctctccaacaataccgcctgcgtcttcaccggtaagggcctttcatttggcggcagtcttattcgcccggaagctaccggctacggtctggtttatttcacagaagcaatgctaaaacgccacggtatgggttttgaagggatgcgcgtttccgtttctggctccggcaacgtcgcccagtacgctatcgaaaaagcgatggaatttggtgctcgtgtgatcactgcgtcagactccagcggcactgtagttgatgaaagcggattcacgaaagagaaactggcacgtcttatcgaaatcaaagccagccgcgatggtcgagtggcagattacgccaaagaatttggtctggtctatctcgaaggccaacagccgtggtctctaccggttgatatcgccctgccttgcgccacccagaatgaactggatgttgacgccgcgcatcagcttatcgctaatggcgttaaagccgtcgccgaaggggcaaatatgccgaccaccatcgaagcgactgaactgttccagcaggcaggcgtactatttgcaccgggtaaagcggctaatgctggtggcgtcgctacatcgggcctggaaatggcacaaaacgctgcgcgcctgggctggaaagccgagaaagttgacgcacgtttgcatcacatcatgctggatatccaccatgcctgtgttgagcatggtggtgaaggtgagcaaaccaactacgtgcagggcgcgaacattgccggttttgtgaaggttgccgatgcgatgctggcgcagggtgtgatttaa

SEQ ID NO: 17 - DNA, 861 bp, synthetic construct
  source 1..861: mol_type = other DNA; note = Homeobox fusion protein, Arabidopsis HB-17 N terminus residues 1 to 91 fused to soybean HB-17 C terminus residues 20-213
atgataaaactactatttacgtacatatgcacatacacatataaactatatgctctatatcatatggattacgcatgcgtgtgtatgtataaatataaaggcatcgtcacgcttcaagtttgtctcttttatattaaactgagagttttcctctcaaactttaccttttcttcttcgatcctagctcttaagaaccctaataattcattgatcaaaataatggcgattttgccggaaaactcttcaaacttggatcttactatctccgttccaggttttgcttcttcaccaacccttcttccctcatcatctgtgaaagaattggacataaatcaagtacctcttgaagaagattggatggcatcaaacatggaagatgaagaagaaagcagcaatggagaacctcctcgaaagaaactccgtctcacaaaggaacaatctcttctccttgaagaaagctttagacaaaaccacacgttgaacccaaagcagaaagagtctttggcaatgcaactgaagctgcgaccaaggcaagtggaggtgtggtttcagaaccgtagggccaggagcaagctgaagcagacagagatggagtgcgagtacctcaagaggtggttcggttccctcacagagcagaaccggaggctccagagggaagtggaggagctgcgagccattaaggtgggcccacccaccgtgatctcccctcactcctgcgaaccgctcccggcctccacactttccatgtgtccccgctgcgagcgtgtcacctccaccgccgacaaaccgccctccgccgcggccactttgtccgctaaagtgccgccaactcaatcccgccaaccctccgcggcctgttag

SEQ ID NO: 18 - DNA, 1038 bp, Glycine max
  source 1..1038: mol_type = other DNA
atgtgtggaggagctatcatctctgatttcattccggcggcggcgatcgccgggtctcgccgcctgaccgccgattacctgtggccggatttgaagaagcggaagtctgacttggacgttgacttcgaggctgatttcagggattttaaagacgattctgatatcgacgacgacgacgacgatcaccaagtcaagccctttgctttcgccgcctcttctcgtctgtctacggcagcgaaatctgtggcattccaaggtcgggctgagatatctgcaaatagaaagaggaagaatcagtataggggaatccgtcaacgcccttggggaaaatgggcagctgagattcgggatccaagaaagggggttcgtgtctggcttggaaccttcaacactgctgaagaagctgcaagagcttatgatgctgaagcacggaggattcgtggcaagaaagccaaggtgaattttcctgaggcaccaggtacttcttctgtaaaacgttccaaggtaaatccacaggaaaatcttaagactgttcagcccaatctgggtcacaagttcagtgctggcaacaatcacatggatctggtggaacagaaacccctagttagccagtatgctaacatggcttccttccctggcagtggaaatgggctaagatcccttccttcgtctgatgatgcaaccctttacttcagttcagatcaagggagtaattcatttgattatgctcctgagatctcatccatgctttctgctcctttggattgtgaatctcattttgtgcaaaatgccaaccagcagcagcctaactctcagaatgtggtatctattgaagatgattctgcaaagacactctctgaggagcttgtggatattgaatctgagttgaagttctttcagatgccttatcttgaagggagctggggcgatacttcattggaatccttgctatctggtgacacaactcaggatggtggaaacctcatgaacctttggtgctttgatgacattccttccatggctggtggagttttctga

SEQ ID NO: 19 - DNA, 774 bp, Oryza sativa
  source 1..774: mol_type = other DNA
atgtcgatgggaccagcagccggagaaggatgtggcctgtgcggcgccgacggtggcggctgttgctcccgccatcgccacgatgatgatggattccccttcgtcttcccgccgagtgcgtgccaggggatcggcgccccggcgccaccggtgcacgagttccagttcttcggcaacgacggcggcggcgacgacggcgagagcgtggcctggctgttcgatgactacccgccgccgtcgcccgttgctgccgccgccgggatgcatcatcggcagccgccgtacgacggcgtcgtggcgccgccgtcgctgttcaggaggaacaccggcgccggcgggctcacgttcgacgtctccctcggcggacggcccgacctggacgccgggctcggcctcggcggcggcagcggccggcacgccgaggccgcggccagcgccaccatcatgtcatattgtgggagcacgttcactgacgcagcgagctcgatgcccaaggagatggtggccgccatggccgatgttggggagagcttgaacccaaacacggtggttggcgcaatggtggagagggaggccaagctgatgaggtacaaggagaagaggaagaagaggtgctacgagaagcaaatccggtacgcgtccagaaaagcctatgccgagatgaggccccgagtgagaggtcgcttcgccaaagaagctgatcaggaagctgtcgcaccgccatccacctatgtcgatcctagtaggcttgagcttggacaatggttcagatag

SEQ ID NO: 20 - DNA, 2916 bp, Zea mays
  source 1..2916: mol_type = other DNA
atgatcgtgcaacccatcgagctacgcgcgtggactgccttccctgggtcggcgcaggaggggatcggaaggatggcggcgtcggtttccagggccatctgcgttcagaagccgggctcaaaatgcaccagggacagggaagcgacctccttcgcccgccgatcggtcgcagcgccgaggcccccgcacgccaaagccgccggcgtcatccgctccgactccggcgcgggacggggccagcattgctcgccgctgagggccgtcgttgacgccgcgccgatacagacgaccaaaaagagggtgttccacttcggcaagggcaagagcgagggcaacaagaccatgaaggaactgctgggcggcaagggcgcgaacctggcggagatggcgagcatcgggctgtcggtgccgccggggttcacggtgtcgacggaggcgtgccagcagtaccaggacgccgggtgcgccctccccgcggggctctgggccgagatcgtcgacggcctgcagtgggtggaggagtacatgggcgccaccctgggcgatccgcagcgcccgctcctgctctccgtccgctccggcgccgccgtgtccatgcccggcatgatggacacggtgctcaacctggggctcaacgacgaagtggccgccgggctggcggccaagagcggggagcgcttcgcctacgactccttccgccgcttcctcgacatgttcggcaacgtcgtcatggacatcccccgctcactgttcgaagagaagcttgagcacatgaaggaatccaaggggctgaagaacgacaccgacctcacggcctctgacctcaaagagctcgtgggtcagtacaaggaggtctacctctcagccaagggagagccattcccctcagaccccaagaagcagctggagctagcagtgctggctgtgttcaactcgtgggagagccccagggccaagaagtacaggagcatcaaccagatcactggcctcaggggcaccgccgtgaacgtgcagtgcatggtgttcggcaacatggggaacacttctggcaccggcgtgctcttcactaggaaccccaacaccggagagaagaagctgtatggcgagttcctggtgaacgctcagggtgaggatgtggttgccggaataagaaccccagaggaccttgacgccatgaagaacctcatgccacaggcctacgacgagcttgttgagaactgcaacatcctggagagccactataaggaaatgcaggatatcgagttcactgtccaggaaaacaggctgtggatgttgcagtgcaggacagggaaacgtacgggcaaaagtgccgtgaagatcgccgtggacatggttaacgagggccttgttgagccccgctcagcgatcaagatggtagagccaggccacctggaccagcttctccatcctcagtttgagaacccgtcggcgtacaaggatcaagtcattgccactggtctgccagcctcacctggggctgctgtgggccaggttgtgttcactgctgaggatgctgaagcatggcattcccaagggaaagctgctattctggtaagggcggagaccagccctgaggacgttggtggcatgcacgctgctgtggggattcttacagagaggggtggcatgacttcccacgctgctgtggtcgcacgtgggtgggggaaatgctgcgtctcgggatgctcaggcattcgcgtaaacgatgcggagaagctcgtgacgatcggaggccatgtgctgcgcgaaggtgagtggctgtcgctgaatgggtcgactggtgaggtgatccttggaaagcagccgctttccccaccagcccttagtggcgatctgggaactttcatggcctgggtggatgatgttagaaagctcaaggtcctggctaacgccgatacccctgatgatgcattgactgcgcgaaacaatggggcacaaggaattggattatgccggacagagcacatgttctttgcttcagacgagaggattaaggctgtcaggcagatgattatggctcccacgcttgagctgaggcagcaggcgctcgaccgtctcttgccgtatcagaggtctgacttcgaaggcattttccgtgctatggatggactcccggtgaccatccgactcctggaccctcccctccacgagttccttccagaagggaacatcgaggacattgtaagtgaattatgtgctgagacgggagccaaccaggaggatgccctcgcgcgaattgaaaagctttcagaagtaaacccgatgcttggcttccgtgggtgcaggcttggtatatcgtaccctgaattgacagagatgcaagcccgggccatttttgaagctgctatagcaatgaccaaccagggtgttcaagtgttcccagagataatggttcctcttgttggaacaccacaggaactggggcatcaagtgactcttatccgccaagttgctgagaaagtgttcgccaatgtgggcaagactatcgggtacaaagttggaacaatgattgagatccccagggcagctctggtggctgatgagatagcggagcaggctgaattcttctccttcggaacgaacgacctgacgcagatgacctttgggtacagcagggatgatgtggggaagttcattcccgtctatcctgctcagggcatcctccaacatgaccccttcgaggtcctggaccagaggggagtgggcgagctggtgaagtttgctacagagaggggccgcaaagctaggcctaacttgaaggtgggcatttgtggagaacacggtggagagccttcctctgtggccttcttcgcgaaggctgggctggattacgtttcttgctcccctttcagggttccgattgctaggctagctgcagctcaggtgcttgtctag

SEQ ID NO: 21 - DNA, 792 bp, Zea mays
  source 1..792: mol_type = other DNA
atgttggagttgcgtctggtgcaggggagcctcctcaagaaggtcttggaggcgatccgcgagctggtcacggacgccaacttcgactgctccgggaccgggttctcgctgcaggccatggactcgagccacgtcgcgctcgtcgcgctgctccttcgcgccgagggcttcgagcactaccgctgcgaccgcaacctctccatgggcatgaacctcaacaacatggccaagatgctccgctgcgctggcaacgaggacatcatcaccatcaaggccgacgacggctccgacaccgtcactttcatgttcgagtcgcccaagcaagataagatcgcggatttcgagatgaaactgatggacattgatagcgagcacctcggaatcccggattccgagtaccaggccatcgtccgcatgccttctgctgagtttatgaggatctgcaaagatcttagcagcatcggagacacagtcgtcatctcggtgactaaggagggcgtgaagttctccacatctggagaaattgggagtgcaaacattgtctgcaggcagaaccaaactattgacaagccagaagaggctaccattatagagatgcaggagccggtttccctgacctttgccctgcggtacatgaactccttcaccaaggcgtcttcactgtctgagcaagtcactatcagcctgtcgtccgagcttccagtggtggtcgagtacaagatcgctgagatgggttacattagattttacctggcccccaagatcgacgatgacgaggagatgaagccctag

SEQ ID NO: 22 - DNA, 2670 bp, Zea mays
  source 1..2670: mol_type = other DNA
atggcgaagtcgagcgcggacgacgccgagctgcggcgcgcgtgcgccgcggccgtggcggcgtcaggcgcgcgcggggaggaggtggccttctccatccgcgtcgccaagggccgcggcatcttcgagaagctcggcaggctcgccaagccccgtgtcctcgcgctcaccgttaaacaatcatcaagaggcgaggcgaacaaagcttttctccgagtattaaaatattcatctggggcagtgctcgagccagccaaactttacaagctgaaacatctaacaaaggttgaggttatttccaatgatcccagtggttgtacatttgttctgggatttgataaccttaggagtcagagtgttgccccgcctcaatggacaatgcgtaacattgatgacagaaatcgtctacttttttgtattctgaatatgtgcaaggagatactcagttatcttccaaaagttgttggaattgatattgtggagctagctctttgggcgaaggaaaacacactgaccatagataatcaagtgagtacccaagatggtcaagaaacatcagttgctactcaaaccgagaggaaagtaacagtaactgttgaaaatgatctcgtgtcccaagcaaaagaggaggaagaagacatggaggcacttcttgacacgtatgttatgggaataggtgaagcagatgcattctctgagagattgaagcaggagcttgtggctttggaggctgcaaatgtatatcaattactggaaagcgagcctttaatagaagaggtcttgcagggtctggatgctgctagtgcaactgtagatgatatggatgagtggttacggattttcaatctgaagctgaggcacatgagagaagatattgcatcgattgaatcacgtaacaatggcttggagatgcagtctgtaaacaacaaagggcttatggaagagctagataaattgcttgagcgtctgcggattccacaagagtttgcagcatcattaactggaggctcatttgaagaatcacggatgctgaaaaatgttgaggcatgtgaatggttgacaggggccattcgcagtcttgaagttcctaatttggacccatgctatgtcaacatgcgcgctgttagagagaaaaaggcagagctggagaaactgaaaacaacttttgttcgacgagcatcagagtttttgcggaactacttctccagtttggtagacttcatgatcagtgacaaaagctacttttcacagcgtggacaattgaagcggcctgatcatgcagaccttaggtataaatgcaggacatatgcccgacttctgcagcacttaaagagtctggacaagagctgcttaggtcccttaaggaaggcatactgtcattcccttaatttactactacgacgagaggcacgtgaatttgccaatgaacttcgtgcaagtacaaaagcaccgaagaatcctgctgtttggcttgaaggttctggcggctctggtcataatggaagcagttctgatacttcacaggtctcagatgcatactcaaagatgcttacaatatttatcccgcttcttgtggatgagagttccttttttgcacattttatgtgctttgaagtacctgcacttgttccagctggttctcctaatgctaataagagcaaatccggaggaaatgacccggatgatgatctgggtcttatggatccagatggcaacgatctgaaacctgatagtacctctgctgaactgggtacattgaatgaagctcttcaagaattactcgatggaatccaggaagacttctatgcggtcgtagattgggcttataaaattgaccccttgcgttgcatctcaatgcatgggattacagagcgttacctttctggacagaaagctgatgctgcaggatttgttcgcaaactacttgatgacttggaatcaagaatatcagtacagttcagccggtttattgatgaagcatgccatcaaattgagcgtaacgaaagaaatgtgcggcaaactggaattctagcctacattccaagatttgctgtccttgcatcacgtatggaacaatatatccaagggcagtccagggatttaattgataaagcatacacaaagctagttagcacaatgttcgcaactttggagaaaattgcacagagtgatcctaaaactgctgatattgtgctgattgagaattatgctgctttccagaacagtctttatgacttggctaatgttgtgccaacgcttgcaaagttctaccatcaagccagtgaatcatatgaactagcttgcactcgccatatcagctcactcatttatctgcaatttgagaggttatttcaattcaaccggaaagttgacgaattgacttacactattgctgctgaggagataccgtttcagctgggattatcaaaaactgacctaaggagggtactgaaatccagcttatctgggattgataagtcgattagcgccatgtacaggaggttacagaagacacttacctccgatgagttgtttccttcgctgtgggacaagtgcaagaaggagtttttggacaaatacgagagttttgttcagatggtgacacggatatatgggaatgagccgatcatgtcagtcaacgaaatgaaagacgtccttgctggtttttag

SEQ ID NO: 23 - DNA, 1179 bp, Zea mays
  source 1..1179: mol_type = other DNA
atgggcactcaaatagtaagggaacaaacgagtggtagcctcactgccattgttgtggacgaaaatctatgccatgccagggctgcaagctgcatgcttgccaacctccagtgtaaagtgattgtgtatgcgagtcctgttgatgcgctcaagtttcttaaggatcaccaaagagatactgattttgcgttagtggaagtcaacatgaaagagatgcatggttttcagttccttgacatgtccaggaagttgcacaaaagccttcaagtgatcatgatgtcagctgatacgacatggcccacgatgaagagaagtgtcgagcttggtgcacgtttcttgattaaaaagcctcttgatgcaaatacgatgaataacctgtggcagcatcttgacctaaaatttcaacggaccgacaagatcaaggccttattcccaggtattgaagggaaaacggggaatgcatttgaggaagggaccaacaagcagaagggaactcacctgatgtggactccattcctgcagaggaagtttttgcaagctgttgaactacttggggaagacgcatctccgaagaagatacagttgctcatgaatgtgaactccgttagtcgcaaacagatctctgctcatcttcagaaacaccgaaagaaggtcgagaaagaactacgtaattcaaatgcaaacaactccagccatggcattggcggcgcctcgaattcacggccttcgaggatttttgaaataagccatggcagattccagtacaatcgtcccgacgtccaaccagaacatagatctgatgaatccgtatccgtggagcagacagaaaccatcgaggaaacacaaagcaacaggttgtatgaagcgatgcgaagagccttgcagcttgggagtgttttcgaggaaccacagttgcccaatgatcctcctgctggtaaagatgcaagagaagttgaagaagtcgaaatgacgatgagggatggtaattatcgagatgctggcacagatgcatttggtgataagaatgaggcatctggaactcacagttcagatggtaataacgcgaaggtcaagagcaaggatgattctgctgacaagctggtttcttgtcacgatgagctgcgaccagttgtgacgcttgtgacctattcagactcggaagatggtgaaacgctctag

SEQ ID NO: 24 - DNA, 768 bp, Zea mays
  source 1..768: mol_type = other DNA
atggggaggggacgagttgagctcaagcggatcgagaacaagatcaaccgccaggtcaccttctccaagcgccgcaacggcctgctcaagaaggcctacgagctctccgtgctctgcgacgccgaggtcgcgctcatcatcttctccagccgcggcaagctctacgagttcggcagcgccggcataacaaaaactttagaaaggtaccaacattgctgctacaatgctcaagattccaatggcgcactctctgaaactcagagctggtaccaggaaatgtcaaaactgagggcaaaattcgaggccttgcagcgcactcagaggcacttgcttggggaggaacttggcccactgagtgtgaaggagttgcagcagctagagaaacagctcgaatgtgctttgtcacaggcaagacagagaaagacacaacttatgatggagcaagtggaagagctccgcagaaaggagcgacacctgggagaaatgaacaggcaactcaaacacaagcttgaagctgaaggttgtagcaactacagaaccctgcagcatgcagcctggccagctcccggcagcaccatggtggagcatgacggcgccacctatcatgtgcatccaacaactgctcaatcggttgcaatggactgtgaacccactctgcaaatcgggtaccctcctcatcaccagtttctgccttccgaggcagccaataatatcccaaggagcccccctggaggcgagaacaacttcatgctgggatgggttctttag

SEQ ID NO: 25 - DNA, 600 bp, Zea mays
  source 1..600: mol_type = other DNA
atggctgaggaggaggccaagaaggtggaggtggaggtcaccaaggagcccgaggcggcagcgaaggaggacgtagccgatgacaaggccgtcatccccgcgaccgacccgccgccgccgccgccgccggccgacgactccaaggccctggccatcgtcgagaaagttgcagatgaacctgctcccgagaagcctgcccctgcgaagcaagggggctccaatgacagggatctcgctcttgcaagggtggaaacagagaagaggaactctttgatcaaagcttgggaagagaatgagaagacaaaagctgagaacaaggctgctaagaaagtatccgctattctttcatgggaaaacacaaagaaagcaaacatagaagctgaactgaagaagattgaggaacaactggaaaagaagaaggctgaatatgcagagaagatgaagaacaaggttgcaatgatacacaaggaagccgaagagaagcgagcgatggtggaggcaaaacgcggtgaggaggtcctgaaggccgaggagatggctgccaagtaccgggccaccggccacgctcctaagaagctcatcggttgcttcggagcctag

SEQ ID NO: 26 - DNA, 537 bp, Zea mays
  source 1..537: mol_type = other DNA
atggcggaagctccggcgagccctggcggcggcggcgggagccacgagagcgggagccccaggggaggcggaggcggtggcagcgtcagggagcaggacaggttcctgcccatcgccaacatcagtcgcatcatgaagaaggccatcccggctaacgggaagatcgccaaggacgctaaggagaccgtgcaggagtgcgtctccgagttcatctccttcatcactagcgaagcgagtgacaagtgccagagggagaagcggaagaccatcaatggcgacgatctgctgtgggccatggccacgctggggtttgaagactacattgaacccctcaaggtgtacctgcagaagtacagagagatggagggtgatagcaagttaactgcaaaatctagcgatggctcaattaaaaaggatgcccttggtcatgtgggagcaagtagctcagctgcacaagggatgggccaacagggagcatacaaccaaggaatgggttatatgcaaccccagtaccataacggggatatctcaaactga

SEQ ID NO: 27 - DNA, 642 bp, Zea mays
  source 1..642: mol_type = other DNA
atgtcgagcggcagcggcagcgggaataagcgcgccgcggcggagaggtcggccggcgccggcgccggtagcggcgacgaggacgacgacggtgccgcacgcaagaagctgcggctgtccaaagaccaggccgccgtgctcgaggagtgcttcaagacgcaccacacgctcactccgaagcagaaggtggcgctggccagcagcctgggcctccggccgcggcaggtggaggtgtggttccagaaccggcgcgcccggaccaagctgaagcagacggaggtggactgcgagtacctcaagcgctggtgcgagcagctcgccgaggagaaccgccgcctgggcaaggaggtcgccgagctcagggcgctcagcgccgcgccggcggccccgctcaccaccctcacgatgtgcctctcctgccggcgcgtcgcctcctcgtccccgtcgtcgtcgtcgtcgcccaggcctagcatccccggcgccgcagctgccagtggcgggagcatggcctctccggcggcggcggcgacgttgcccgcccacaggcagttcttctgcgggttcagagacgccggggcggcggccgcggcgtacgggacagcctcggcggggctcgcgaagcctgtcagggctgccagatag

SEQ ID NO: 28 - DNA, 1272 bp, synthetic construct
  source 1..1272: mol_type = other DNA; note = corn putative glutamine synthetase (with C299A mutation)
atggcgcaggcggtggtgccggcgatgcagtgccgggtcggagtgaaggcggcggcggggagggtgtggagcgccggcaggactaggaccggccgcggcggcgcctcgccggggttcaaggtcatggccgtcagcacgggcagcaccggggtggtgccgcgcctcgagcagctgctcaacatggacaccacgccctacaccgacaaggtcatcgccgagtacatctgggtcggaggatctggaatcgacatccgaagcaaatcaaggacgatttcgaaacccgtggaggatccctcggaactaccaaaatggaactacgatggatctagcacaggacaagccccgggagaagacagtgaagtcattctatacccccaggctatcttcaaggacccattccgaggtggcaacaacgttttggttatctgtgacacctacacgccacagggggaaccccttccaactaacaaacgccacagggctgcgcaaattttcagtgacccaaaggtcgctgaacaagtgccatggtttggcatagagcaagagtacactttgctccagaaagatgtaaattggcctcttggttggcctgttggaggcttccctggtccccagggtccatactactgtgccgtaggagccgacaaatcatttggccgtgacatatcagatgctcactacaaggcatgcctctacgctggaatcaacattagtggaacaaacggggaggtcatgcctggtcagtgggagtaccaagttggacctagtgttggtattgaagcaggagatcacatatggatttcgagatacattctcgagagaatcacagagcaagctggggttgtccttacccttgatccaaaaccaattcagggtgactggaacggagctggcgcacacacaaattacagcacaaagaccatgcgcgaagacggcgggtttgaagagatcaagagagcaatcctgaacctttctctgcgccatgatctgcatattagtgcatacggagaaggaaatgaaagaagactgactgggaaacatgagactgcgagcatcggaacgttctcatggggtgtggcaaaccgcggctgctctatccgtgtggggcgggataccgaggcaaaagggaaaggttacctggaagaccgtcggccggcatcaaacatggacccgtacattgtgacggggctactggccgagaccacgatcctctggcagccatccctcgaggcggaggctcttgccgccaagaagctggcgctgaaggtgtga

SEQ ID NO: 29 - DNA, 1785 bp, Zea mays
  source 1..1785: mol_type = other DNA
atggtgaagaggagcaagaagagcaagagcaagcgagtcacgctgcggcagaagcacaaggtgcagcgcaaggtgaaggagcaccaccgcaagaagcgcaaggaggccaagaaggcgggcaaggccggccagcggaggaaggtcgagaaggaccccgggatccccaacgagtggcccttcaaggagcaggagctcaaggcgctcgaggcccgacgcgcgcaggcgctccaggagctcgagctcaagaagcaggcgcgcaaggagagggctcagaagaggaaggcaggattgcttgaggacgaggacattgctagtttggcatctgcagcttccgcacagggcagtgagtttgcagcaaaggagaatgctcccttattagtggcaaagatcaacgatcattcagagaggtctttttacaaggagcttgttaaagtaattgaagcttccgatgtgattatggaggttcttgatgcaagggatccgctgggcacccgttgcattgacatggaaaagatggttaggaaggctgatccaagtaaacggattgtactacttctcaacaagatagatcttgttcccaaggaggcggcagagaaatggcttacatatctaagagaagaattgccaacagttgcttttaagtgcaatacccaggagcagaggaccaagctgggatggaaatcttcaaaattagataaaacaagcaacatcccacaaagcagtgattgtcttggtgccgagaatctaatcaaattgcttaagaattattccagaagccatgagctcaaactgacaattacggtgggtattgttggacttcctaatgttggcaagagcagtctgatcaacagcctgaagaggtcccgagtggtcaatgtcggttctactccagggattactagatcaatgcaagaagttcaattagacaagaaggtaaagttgttggattgtcctggtgttgttatgctcaaatcttccaacagtggtgtgtctgtagcccttcgaaactgcaaaaaggttgagaaaatagaagatccagtcgctcctgttaagcagatcctaagtatttgcccacatgagaagttgctgtctctatacaaggttccaaactttggttcagttgatgattttcttcagaaagtagcaactgtacgtgggaaattgaaaaagggtggtgtagtggatgttgaagctgcagcgagaattgtgcttcatgactggaatgaaggtaaaataccatattttacactgccgcctaaaagagatgctggggaggactcagatgcagtgattatatccgaggatgggaaagaatttaatatcgatgagatttacaaagctgaatcttcatatattggtggtttgaagtccattgaggagttccatcatattgagattcctcctaatgctccactggcgatcgatgaagagatgctagaggatggtggcaagaaaccaagcgaagccatccaggaaagccgggacagggaggagcaaatgcccgacgtgaaggactcaggaggaagcaaggcagccagcgccagcacgcagaacgacaagttgtacaccgccgagggcgtactcgaccctcgcaagagcaaagcagagaagaaacggcgcaaggcgagcaggcccagcgcgctgaacgacatggacgcggactacgatttcaaggtggattatcggatggaggacggtgggagcgagggtgcacacgcggatgatgaagatggcggagatggatccgaggataatgaaccaatgaccggtgtcgatgatgcatga

SEQ ID NO: 30 - protein, 305 aa, Agrobacterium tumefaciens
MASPKHFLDLSAVGPQDLRTILDDARARKVATKAGTAEKPLAGKMLAMIFEKPSTRTRVSFDVGMRQLGGETLFLSGTEMQLGRAETIGDTAKVLSRYVDAIMIRTTDHSRLLELAEHATVPVINGLTDDTHPCQIMADIMTFEEHRGPVKGKTIAWTGDGNNVLHSFVEGSARFGYRMTMAVPMGSEPHDKFMNWARNNGGEIALYHDADKAVAGADCVVTDTWVSMNQEHKARGHNIFQPYQVNEALMAKAQKDALFMHCLPAHRGEEVTDAVIDGPQSVVFDEAENRLHAQKSVIAWCMGVI

SEQ ID NO: 31 - protein, 214 aa, Arabidopsis thaliana
MVRGKTQMKRIENATSRQVTFSKRRNGLLKKAFELSVLCDAEVSLIIFSPKGKLYEFASSNMQDTIDRYLRHTKDRVSTKPVSEENMQHLKYEAANMMKKIEQLEASKRKLLGEGIGTCSIEELQQIEQQLEKSVKCIRARKTQVFKEQIEQLKQKEKALAAENEKLSEKWGSHESEVWSNKNQESTGRGDEESSPSSEVETQLFIGLPCSSRK

SEQ ID NO: 32 - protein, 1006 aa, Arabidopsis thaliana
MGEQQISVTVPRDRTDEQAILALKKGAQLLKCRRRGNPKFCPFKLSMDEKYLIWYSGEEERQLRLSSVITIVRGQITPNFQKQAQSDRKEQSFSLIYANGEHTLDLICKDKAQADSWFKGLRAVITKHHNIRNSVNHRSSRGAQSCINSPAGFMRRKQNLGLLEETPDVTQIRSLCGSPSTLLEERCLSNGLSCSSDSFAESDALGPVSSYYETDYDFRNSDCDRSTGSELCRFSSQRFAASPPLSIITQPVTRSNVLKDIMIWGAITGLIDGSKNQNDALSPKLLESATMFDVQSISLGAKHAALVTRQGEVFCWGNGNSGKLGLKVNIDIDHPKRVESLEDVAVRSVACSDHQTCAVTESGELYLWGIDGGTIEQSGSQFLTRKISDVLGGSLTVLSVACGAWHTAIVTSSGQLFTYGSGTFGVLGHGSLESVTKPKEVESLRRMKVISVSCGPWHTAAIVETANDRKFYNAKSCGKLFTWGDGDKGRLGHADSKRKLVPTCVTELIDHDFIKVSCGWTLTVALSISGTVYTMGSSIHGQLGCPRAKDKSVNVVLGNLTRQFVKDIASGSHHVAVLTSFGNVYTWGKGMNGQLGLGDVRDRNSPVLVEPLGDRLVESIACGLNLTAAICLHKEISLNDQTACSSCKSAFGFTRRKHNCYNCGLLFCNACSSKKAVNASLAPNKSKLSRVCDSCFDHLWSITEFSRNVKMDNHTPRMQMVTRRVSEDLTEKQSENEMQNLPQANRSSDGQPRWGQVSGPSLFRFDKISTSSSLNLSVSARRTSSTKISTSSESNKILTEEIERLKAVIKNLQRQCELGNEKMEECQQELDKTWEVAKEEAEKSKAAKEIIKALASKLQANKEKPSNPLKTGIACNPSQVSPIFDDSMSIPYLTPITTARSQHETKQHVEKCVTKSSNRDSNIKLLVDASPAITRTGYLQNETQDSSAEQVEQYEPGVYITFTALPCGQKTLKRVRFSRKRFSEKEAQRWWEEKQVLVYNKYDAEI

SEQ ID NO: 33 - protein, 729 aa, Arabidopsis thaliana
MRIFSTFVFHRRQQIFNLRQFQTTTILRNPISIAPIQIPMDATEQSLRQSLSEKSSSVEAQGNAVRALKASRAAKPEIDAAIEQLNKLKLEKSTVEKELQSIISSSGNGSLNREAFRKAVVNTLERRLFYIPSFKIYSGVAGLFDYGPPGCAIKSNVLSFWRQHFILEENMLEVDCPCVTPEVVLKASGHVDKFTDLMVKDEKTGTCYRADHLLKDYCTEKLEKDLTISAEKAAELKDVLAVMEDFSPEQLGAKIREYGITAPDTKNPLSDPYPFNLMFQTSIGPSGLIPGYMRPETAQGIFVNFKDLYYYNGKKLPFAAAQIGQAFRNEISPRQGLLRVREFTLAEIEHFVDPENKSHPKFSDVAKLEFLMFPREEQMSGQSAKKLCLGEAVAKGTVNNETLGYFIGRVYLFLTRLGIDKERLRFRQHLANEMAHYAADCWDAEIESSYGWIECVGIADRSAYDLRAHSDKSGTPLVAEEKFAEPKEVEKLVITPVKKELGLAFKGNQKNVVESLEAMNEEEAMEMKATLESKGEVEFYVCTLKKSVNIKKNMVSISKEKKKEHQRVFTPSVIEPSFGIGRIIYCLYEHCFSTRPSKAGDEQLNLFRFPPLVAPIKCTVFPLVQNQQFEEVAKVISKELASVGISHKIDITGTSIGKRYARTDELGVPFAITVDSDTSVTIRERDSKDQVRVTLKEAASVVSSVSEGKMTWQDVWATFPHHSSAAADE

SEQ ID NO: 34 - protein, 588 aa, Arabidopsis thaliana
MSYDEHTSSSFTYISQMGVWWIVLVVAVLTHTASAAVREYHWEVEYKYWSPDCKEGAVMTVNGEFPGPTIKAFAGDTIVVNLTNKLTTEGLVIHWHGIRQFGSPWADGAAGVTQCAINPGETFTYNFTVEKPGTHFYHGHYGMQRSAGLYGSLIVDVAKGKSERLRYDGEFNLLLSDWWHEAIPSQELGLSSKPMRWIGEAQSILINGRGQFNCSLAAQFSNNTSLPMCTFKEGDQCAPQILHVEPNKTYRIRLSSTTALASLNLAVQGHKLVVVEADGNYITPFTTDDIDIYSGESYSVLLTTDQDPSQNYYISVGVRGRKPNTTQALTILNYVTAPASKLPSSPPPVTPRWDDFERSKNFSKKIFSAMGSPSPPKKYRKRLILLNTQNLIDGYTKWAINNVSLVTPATPYLGSVKYNLKLGFNRKSPPRSYRMDYDIMNPPPFPNTTTGNGIYVFPFNVTVDVIIQNANVLKGIVSEIHPWHLHGHDFWVLGYGDGKFKPGIDEKTYNLKNPPLRNTAILYPYGWTAIRFVTDNPGVWFFHCHIEPHLHMGMGVVFAEGLNRIGKVPDEALGCGLTKQFLMNRNRN

SEQ ID NO: 35 - protein, 493 aa, Arabidopsis thaliana
MVLSKTVSESDVSIHSTFASRYVRNSLPRFEMPENSIPKEAAYQIINDELMLDGNPRLNLASFVTTWMEPECDKLMMESINKNYVDMDEYPVTTELQNRCVNMIARLFNAPLGDGEAAVGVGTVGSSEAIMLAGLAFKRQWQNKRKAQGLPYDKPNIVTGANVQVCWEKFARYFEVELKEVNLREDYYVMDPVKAVEMVDENTICVAAILGSTLTGEFEDVKLLNDLLVEKNKQTGWDTPIHVDAASGGFIAPFLYPELEWDFRLPLVKSINVSGHKYGLVYAGIGWVVWRTKTDLPDELIFHINYLGADQPTFTLNFSKGSSQVIAQYYQLIRLGFEGYRNVMDNCRENMMVLRQGLEKTGRFKIVSKENGVPLVAFSLKDSSRHNEFEVAHTLRRFGWIVPAYTMPADAQHVTVLRVVIREDFSRTLAERLVADFEKVLHELDTLPARVHAKMANGKVNGVKKTPEETQREVTAYWKKLLETKKTNKNTIC

SEQ ID NO: 36 - protein, 282 aa, Arabidopsis thaliana
MGTRAQQIPLLEGETDNYDGVTVTMVEPMDSEVFTESLRASLSHWREEGKKGIWIKLPLGLANLVEAAVSEGFRYHHAEPEYLMLVSWISETPDTIPANASHVVGAGALVINKNTKEVLVVQERSGFFKDKNVWKLPTGVINEGEDIWTGVAREVEEETGIIADFVEVLAFRQSHKAILKKKTDMFFLCVLSPRSYDITEQKSEILQAKWMPIQEYVDQPWNKKNEMFKFMANICQKKCEEEYLGFAIVPTTTSSGKESFIYCNADHAKRLKVSRDQASASL

SEQ ID NO: 37 - protein, 460 aa, Arabidopsis thaliana
MTVVGDVAPIPRRNSSTCSNDIAAPLLPECHGDEVAHDEFNGASFSGAVFNLATTIIGAGIMALPATMKILGLGLGITMIVVMAFLTDASIEFLLRFSKAGKNRSYGGLMGGSFGNPGRILLQVAVLVNNIGVLIVYMIIIGDVLAGKTEDGIHHFGVLEGWFGHHWWNGRAAILLITTLGVFAPLACFKRIDSLKFTSALSVALAVVFLIITAGISIMKLISGGVAMPRLLPDVTDLTSFWNLFTVVPVLVTAFICHYNVHSIQNELEDPSQIRPVVRSALMLCSSVYIMTSIFGFLLFGDDTLDDVLANFDTDLGIPFGSILNDAVRVSYALHLMLVFPIVFYPLRINIDGLLFPSARSLSTSNVRFGCLTAGLISVIFLGANFIPSIWDAFQFTGATAAVCLGFIFPASIILKDRHDKATNRDTTLAIFMIVLAVLSNAIAIYSDAYALFKKNAPRE

SEQ ID NO: 38 - protein, 133 aa, synthetic construct
  MUTAGEN 33: note = A33V mutation
  source 1..133: note = Arabidopsis thaliana putative AP2/EREBP transcription factor, with A33V mutation
MDQGGRSSGSGGGGAEQGKYRGVRRRPWGKYAVEIRDSRKHGERVWLGTFDTAEDAARAYDRAAYSMRGKAAILNFPHEYNMGTGSSSTAANSSSSSQQVFEFEYLDDSVLDELLEYGENYNKTHNINMGKRQ

SEQ ID NO: 39 - protein, 357 aa, Arabidopsis thaliana
MVIYHRKVVFTYVRAKRFYHFLNIEMVTDFKSLLPVIDISPLLAKCDDFDMAEDAGVVEVVGKLDRACRDVGFFYVIGHGISDDLINKVKEMTHQFFELPYEEKLKIKITPTAGYRGYQRIGVNFTSGKQDMHEAIDCYREFKQGKHGDIGKVLEGPNQWPGNPQEYKDLMEKYIKLCTDLSRNILRGISLALGGSPYEFEGKMLRDPFWVMRIIGYPGVNQENVIGCGAHTDYGLLTLINQDDDKTALQVKNVDGDWIPAIPIPGSFICNIGDMLTILSNGVYQSTLHKVINNSPKYRVCVAFFYETNFEAEVEPLDIFKEKHPRKETSQVAKRVVYGQHLINKVLTTFANLVENS

SEQ ID NO: 40 - protein, 371 aa, Arabidopsis thaliana
MPVLFSSRSLILSIIVPLLISIALYKLDTFDPAIVPSDAFTSSATSLPPLINDEFLTGAEFIGVGLLNIPEDIAYHKESNLIYTGCVDGWVKRVKVADSVNDSVVEDWVNTGGRPLGIAFGIHGEVIVADVHKGLLNISGDGKKTELLTDEADGVKFKLTDAVTVADNGVLYFTDASYKYTLNQLSLDMLEGKPFGRLLSFDPTTRVTKVLLKDLYFANGITISPDQTHLIFCETPMKRCSKYYISEERVEVFTQSLPGYPDNIRYDGDGHYWIALPSGVTTLWNISLKYPFLRKLTAMVAKYGVDLMFMENAGVLQVDLDGNPIAYYHDPKLSHIATCDKIGKYLYCGSLSQSHILRLDLLKYPAQNKKL

SEQ ID NO: 41 - protein, 468 aa, Arabidopsis thaliana
MGRVRKSDFGSIVLVLCCVLNSLLCNGGITSRYVRKLEATVDMPLDSDVFRVPCGYNAPQQVHITQGDVEGKAVIVSWVTQEAKGSNKVIYWKENSTKKHKAHGKTNTYKFYNYTSGFIHHCPIRNLEYDTKYYYVLGVGQTERKFWFFTPPEIGPDVPYTFGLIGDLGQSYDSNITLTHYENNPTKGQAVLFVGDISYADTYPDHDNRRWDSWGRFAERSTAYQPWIWTTGNHELDFAPEIGENRPFKPFTHRYRTPYRSSGSTEPFWYSIKRGPAYIIVLASYSAYGKYTPQYQWLEEEFPKVNRTETPWLIVLMHSPWYNSYDYHYMEGETMRVMYEAWFVKYKVDVVFAGHVHAYERSERVSNIAYNVVNGICTPVKDQSAPVYITIGDGGNIEGLATKMTEPQPKYSAFREASFGHAIFSIKNRTHAHYGWHRNHDGYAVEGDRMWFYNRFWHPVDDSPSCNS

SEQ ID NO: 42 - protein, 499 aa, Arabidopsis thaliana
MLLKPGNKLVSPETSHHRDSASNSSNHKCQQQKPRKDKQKQVEQNTKKIEEHQIKSESTLLISNHNVNMSSQSNNSESTSTNNSSKPHTGGDIRWDAVNSLKSRGIKLGISDFRVLKRLGYGDIGSVYLVELKGANPTTYFAMKVMDKASLVSRNKLLRAQTEREILSQLDHPFLPTLYSHFETDKFYCLVMEFCSGGNLYSLRQKQPNKCFTEDAARFFASEVLLALEYLHMLGIVYRDLKPENVLVRDDGHIMLSDFDLSLRCSVNPTLVKSFNGGGTTGIIDDNAAVQGCYQPSAFFPRMLQSSKKNRKSKSDFDGSLPELMAEPTNVKSMSFVGTHEYLAPEIIKNEGHGSAVDWWTFGIFIYELLHGATPFKGQGNKATLYNVIGQPLRFPEYSQVSSTAKDLIKGLLVKEPQNRIAYKRGATEIKQHPFFEGVNWALIRGETPPHLPEPVDFSCYVKKEKESLPPAATEKKSKMFDEANKSGSDPDYIVFEYF

SEQ ID NO: 43 - protein, 1552 aa, Chlamydomonas reinhardtii
MEAVATVVANEPKVADMEEILAERDACGVGFIANLKNVQSHTVVKQALTALGCMEHRGACSADDDSGDGAGLMTQIPWKLLKKEMPALNETTTGVGMVFMPNDDALEAQCKQILEQVCAKEGVKVVGWRKVPVNHDIVGRFAKVTEPRIWQVLIEGKSGQVGDELERELFLVRKLVEKAKNAALPAEFAPDFYICTLSSRTIVYKGMLRSAVVGTFFRDLENPDFESAFAIYHRRFSTNTTPKWPLAQPMRVLGHNGEINTLQGNLNWVASREHELSNPIWKGREAELTPLCNAAQSDSANLDNVAELLVRTGTDPQDALMLLVPEAYRNHPDLMKEYPEVVDFYEFYEGLQEGWDGPALLVFSDGKRVGARLDRNGLRPARFWQTKDDMIYVASEVGVLGDAITNAENIVAKGRLGPGQMVCADLEKGIFSETSAISKLVAGRKPYKEWLAASLRRLTDLGESTFLNEPMYDAATMLRLQSAIGMDAENAQMVVESQAQTGVEPTYCMGDDIPLAVLSDKPHMLYDYFKQRFAQVTNPPIDPLREGLVMSLEMRLGARGNLLNPGADSYKQVLLDSPILLESEMQAISTDKVLGSKTFKLFFEAGKPGAMEAALKKLCSDVEAAVKAGCQCVVLSDRPDGGMDAGKAPIPALLATGAVHHHLIRTSLRSDTSIVVDTATCYSTHHAAMLIGFGAHAICPYLGYETSRQWRLSARTQSLIKAGKVPDISVKVAQKNFKKSLEKGVLKILSKMGISLLSCYHGAQIFEAYGLGKDVMDMCFKGTVSRIGGMSLADLQRESESLWAKGFPEKAMTKLEDYGFIQSKPKGEFHSNNQTMAKLLHKAIGLGNGSAADKDAYKAYQQHFADSPVAVLRDCLEFKSDRGPISIDQVEPAAAIMERFCTGGMSLGAISRETHETIAIAMNRIGGKSNSGEGGEDPIRWLHLSDVDGEGKSATASYLRGLRNGDTATSKIKQVASGRFGVTPEYIMNAEQMEIKIAQGAKPGEGGQLPGQKVSPYIAQLRRSKPGVPLISPPPHHDIYSIEDLAQLIYDLHQVNPRAKVSVKLVAEAGIGVVASGVAKANADIIQVSGHDGGTGASPISSIKHAGGPMEMGLAETHQTLVRNELRERVVLRVDGGVRNGRDVLMGALMGADEFGFGTVAMIATGCIMARVCHTNNCPVGVASQREELRARFPGAPEDLVNYFHFVAEEVRAELANMGYRSLDEVIGRADLLKQRSVKLAKTEGLDLSFLTTFAGASGKSSTRRAQEVHDNGPQLDDRILAEPEVMAAIKDHKTVSKAFEIVNVDRSSLGRVAGVIAKHHGDSGFQGKVKLTLTGSGGQSFGCFCVKGLEVKLVGEANDYVGKGMNGGEIAIVPPANSPFKPEEASLVGNTCLYGATGGRLFVNGRAGERFAVRNSLAEAVVEGAGDHCCEYMTGGCVIVLGSVGRNVAAGMTGGLGYFLDEDGSFTDKVNTEIVSVQRVITKAGEAQLRGLLEAHVAHTGSAKAKSLLANWEASLGKFWQLVPPAEKNTAEVNPSVAQPAAAGAKVAVSA

SEQ ID NO: 44 - protein, 449 aa, synthetic construct
  source 1..449: note = Chlorella sorokiniana NADP-specific glutamate dehydrogenase (NADP-GDH), N terminus residues 1 to 74 truncated
MHGIKNPELRQLLTEIFMKDPEQQEFMQAVREVAVSLQPVFEKRPELLPIFKQIVEPERVITFRVSWLDDAGNLQVNRGFRVQYSSAIGPYKGGLRFHPSVNLSIMKFLAFEQIFKNSLTTLPMGGGKGGSDFDPKGKSDAEVMRFCQSFMTELQRHISYVQDVPAGDIGVGAREIGYLFGQYKRITKNYTGVLTGKGQEYGGSEIRPEATGYGAVLFVENVLKDKGESLKGKRCLVSGAGNVAQYCAELLLEKGAIVLSLSDSQGYVYEPNGFTREQLQAVQDMKKKNNSARISEYKSDTAVYVGDRRKPWELDCQVDIAFPCATQNEIDEHDAELLIKHGCQYVVEGANMPSTNEAIHKYNKAGIIYCPGKAANAGGVAVSGLEMTQNRMSLNWTREEVRDKLERIMKDIYDSAMGASREYNVDLAAGANIAGFTKVADAVKAQGAV

SEQ ID NO: 45 - protein, 447 aa, Escherichia coli
MDQTYSLESFLNHVQKRDPNQTEFAQAVREVMTTLWPFLEQNPKYRQMSLLERLVEPERVIQFRVVWVDDRNQIQVNRAWRVQFSSAIGPYKGGMRFHPSVNLSILKFLGFEQTFKNALTTLPMGGGKGGSDFDPKGKSEGEVMRFCQALMTELYRHLGADTDVPAGDIGVGGREVGFMAGMMKKLSNNTACVFTGKGLSFGGSLIRPEATGYGLVYFTEAMLKRHGMGFEGMRVSVSGSGNVAQYAIEKAMEFGARVITASDSSGTVVDESGFTKEKLARLIEIKASRDGRVADYAKEFGLVYLEGQQPWSLPVDIALPCATQNELDVDAAHQLIANGVKAVAEGANMPTTIEATELFQQAGVLFAPGKAANAGGVATSGLEMAQNAARLGWKAEKVDARLHHIMLDIHHACVEHGGEGEQTNYVQGANIAGFVKVADAMLAQGVI

SEQ ID NO: 46 - protein, 286 aa, synthetic construct
  MUTAGEN 1..91: note = source: Arabidopsis HB-17 N terminus residues 1 to 91
  MUTAGEN 92..286: note = source: soybean HB-17 C terminus residues 20-213
  source 1..286: note = Homeobox fusion protein, Arabidopsis HB-17 N terminus residues 1 to 91 fused to soybean HB-17 C terminus residues 20-213
MIKLLFTYICTYTYKLYALYHMDYACVCMYKYKGIVTLQVCLFYIKLRVFLSNFTFSSSILALKNPNNSLIKIMAILPENSSNLDLTISVPGFASSPTLLPSSSVKELDINQVPLEEDWMASNMEDEEESSNGEPPRKKLRLTKEQSLLLEESFRQNHTLNPKQKESLAMQLKLRPRQVEVWFQNRRARSKLKQTEMECEYLKRWFGSLTEQNRRLQREVEELRAIKVGPPTVISPHSCEPLPASTLSMCPRCERVTSTADKPPSAAATLSAKVPPTQSRQPSAAC

SEQ ID NO: 47 - protein, 345 aa, Glycine max
MCGGAIISDFIPAAAIAGSRRLTADYLWPDLKKRKSDLDVDFEADFRDFKDDSDIDDDDDDHQVKPFAFAASSRLSTAAKSVAFQGRAEISANRKRKNQYRGIRQRPWGKWAAEIRDPRKGVRVWLGTFNTAEEAARAYDAEARRIRGKKAKVNFPEAPGTSSVKRSKVNPQENLKTVQPNLGHKFSAGNNHMDLVEQKPLVSQYANMASFPGSGNGLRSLPSSDDATLYFSSDQGSNSFDYAPEISSMLSAPLDCESHFVQNANQQQPNSQNVVSIEDDSAKTLSEELVDIESELKFFQMPYLEGSWGDTSLESLLSGDTTQDGGNLMNLWCFDDIPSMAGGVF

SEQ ID NO: 48 - protein, 257 aa, Oryza sativa
MSMGPAAGEGCGLCGADGGGCCSRHRHDDDGFPFVFPPSACQGIGAPAPPVHEFQFFGNDGGGDDGESVAWLFDDYPPPSPVAAAAGMHHRQPPYDGVVAPPSLFRRNTGAGGLTFDVSLGGRPDLDAGLGLGGGSGRHAEAAASATIMSYCGSTFTDAASSMPKEMVAAMADVGESLNPNTVVGAMVEREAKLMRYKEKRKKRCYEKQIRYASRKAYAEMRPRVRGRFAKEADQEAVAPPSTYVDPSRLELGQWFR

SEQ ID NO: 49 - protein, 971 aa, Zea mays
MIVQPIELRAWTAFPGSAQEGIGRMAASVSRAICVQKPGSKCTRDREATSFARRSVAAPRPPHAKAAGVIRSDSGAGRGQHCSPLRAVVDAAPIQTTKKRVFHFGKGKSEGNKTMKELLGGKGANLAEMASIGLSVPPGFTVSTEACQQYQDAGCALPAGLWAEIVDGLQWVEEYMGATLGDPQRPLLLSVRSGAAVSMPGMMDTVLNLGLNDEVAAGLAAKSGERFAYDSFRRFLDMFGNVVMDIPRSLFEEKLEHMKESKGLKNDTDLTASDLKELVGQYKEVYLSAKGEPFPSDPKKQLELAVLAVFNSWESPRAKKYRSINQITGLRGTAVNVQCMVFGNMGNTSGTGVLFTRNPNTGEKKLYGEFLVNAQGEDVVAGIRTPEDLDAMKNLMPQAYDELVENCNILESHYKEMQDIEFTVQENRLWMLQCRTGKRTGKSAVKIAVDMVNEGLVEPRSAIKMVEPGHLDQLLHPQFENPSAYKDQVIATGLPASPGAAVGQVVFTAEDAEAWHSQGKAAILVRAETSPEDVGGMHAAVGILTERGGMTSHAAVVARGWGKCCVSGCSGIRVNDAEKLVTIGGHVLREGEWLSLNGSTGEVILGKQPLSPPALSGDLGTFMAWVDDVRKLKVLANADTPDDALTARNNGAQGIGLCRTEHMFFASDERIKAVRQMIMAPTLELRQQALDRLLPYQRSDFEGIFRAMDGLPVTIRLLDPPLHEFLPEGNIEDIVSELCAETGANQEDALARIEKLSEVNPMLGFRGCRLGISYPELTEMQARAIFEAAIAMTNQGVQVFPEIMVPLVGTPQELGHQVTLIRQVAEKVFANVGKTIGYKVGTMIEIPRAALVADEIAEQAEFFSFGTNDLTQMTFGYSRDDVGKFIPVYPAQGILQHDPFEVLDQRGVGELVKFATERGRKARPNLKVGICGEHGGEPSSVAFFAKAGLDYVSCSPFRVPIARLAAAQVLV

SEQ ID NO: 50 - protein, 263 aa, Zea mays
MLELRLVQGSLLKKVLEAIRELVTDANFDCSGTGFSLQAMDSSHVALVALLLRAEGFEHYRCDRNLSMGMNLNNMAKMLRCAGNEDIITIKADDGSDTVTFMFESPKQDKIADFEMKLMDIDSEHLGIPDSEYQAIVRMPSAEFMRICKDLSSIGDTVVISVTKEGVKFSTSGEIGSANIVCRQNQTIDKPEEATIIEMQEPVSLTFALRYMNSFTKASSLSEQVTISLSSELPVVVEYKIAEMGYIRFYLAPKIDDDEEMKP

SEQ ID NO: 51 - protein, 889 aa, Zea mays
MAKSSADDAELRRACAAAVAASGARGEEVAFSIRVAKGRGIFEKLGRLAKPRVLALTVKQSSRGEANKAFLRVLKYSSGAVLEPAKLYKLKHLTKVEVISNDPSGCTFVLGFDNLRSQSVAPPQWTMRNIDDRNRLLFCILNMCKEILSYLPKVVGIDIVELALWAKENTLTIDNQVSTQDGQETSVATQTERKVTVTVENDLVSQAKEEEEDMEALLDTYVMGIGEADAFSERLKQELVALEAANVYQLLESEPLIEEVLQGLDAASATVDDMDEWLRIFNLKLRHMREDIASIESRNNGLEMQSVNNKGLMEELDKLLERLRIPQEFAASLTGGSFEESRMLKNVEACEWLTGAIRSLEVPNLDPCYVNMRAVREKKAELEKLKTTFVRRASEFLRNYFSSLVDFMISDKSYFSQRGQLKRPDHADLRYKCRTYARLLQHLKSLDKSCLGPLRKAYCHSLNLLLRREAREFANELRASTKAPKNPAVWLEGSGGSGHNGSSSDTSQVSDAYSKMLTIFIPLLVDESSFFAHFMCFEVPALVPAGSPNANKSKSGGNDPDDDLGLMDPDGNDLKPDSTSAELGTLNEALQELLDGIQEDFYAVVDWAYKIDPLRCISMHGITERYLSGQKADAAGFVRKLLDDLESRISVQFSRFIDEACHQIERNERNVRQTGILAYIPRFAVLASRMEQYIQGQSRDLIDKAYTKLVSTMFATLEKIAQSDPKTADIVLIENYAAFQNSLYDLANVVPTLAKFYHQASESYELACTRHISSLIYLQFERLFQFNRKVDELTYTIAAEEIPFQLGLSKTDLRRVLKSSLSGIDKSISAMYRRLQKTLTSDELFPSLWDKCKKEFLDKYESFVQMVTRIYGNEPIMSVNEMKDVLAGF

SEQ ID NO: 52 - protein, 392 aa, Zea mays
MGTQIVREQTSGSLTAIVVDENLCHARAASCMLANLQCKVIVYASPVDALKFLKDHQRDTDFALVEVNMKEMHGFQFLDMSRKLHKSLQVIMMSADTTWPTMKRSVELGARFLIKKPLDANTMNNLWQHLDLKFQRTDKIKALFPGIEGKTGNAFEEGTNKQKGTHLMWTPFLQRKFLQAVELLGEDASPKKIQLLMNVNSVSRKQISAHLQKHRKKVEKELRNSNANNSSHGIGGASNSRPSRIFEISHGRFQYNRPDVQPEHRSDESVSVEQTETIEETQSNRLYEAMRRALQLGSVFEEPQLPNDPPAGKDAREVEEVEMTMRDGNYRDAGTDAFGDKNEASGTHSSDGNNAKVKSKDDSADKLVSCHDELRPVVTLVTYSDSEDGETL

SEQ ID NO: 53 - protein, 255 aa, Zea mays
MGRGRVELKRIENKINRQVTFSKRRNGLLKKAYELSVLCDAEVALIIFSSRGKLYEFGSAGITKTLERYQHCCYNAQDSNGALSETQSWYQEMSKLRAKFEALQRTQRHLLGEELGPLSVKELQQLEKQLECALSQARQRKTQLMMEQVEELRRKERHLGEMNRQLKHKLEAEGCSNYRTLQHAAWPAPGSTMVEHDGATYHVHPTTAQSVAMDCEPTLQIGYPPHHQFLPSEAANNIPRSPPGGENNFMLGWVL

SEQ ID NO: 54 - protein, 199 aa, Zea mays
MAEEEAKKVEVEVTKEPEAAAKEDVADDKAVIPATDPPPPPPPADDSKALAIVEKVADEPAPEKPAPAKQGGSNDRDLALARVETEKRNSLIKAWEENEKTKAENKAAKKVSAILSWENTKKANIEAELKKIEEQLEKKKAEYAEKMKNKVAMIHKEAEEKRAMVEAKRGEEVLKAEEMAAKYRATGHAPKKLIGCFGA

SEQ ID NO: 55 - protein, 178 aa, Zea mays
MAEAPASPGGGGGSHESGSPRGGGGGGSVREQDRFLPIANISRIMKKAIPANGKIAKDAKETVQECVSEFISFITSEASDKCQREKRKTINGDDLLWAMATLGFEDYIEPLKVYLQKYREMEGDSKLTAKSSDGSIKKDALGHVGASSSAAQGMGQQGAYNQGMGYMQPQYHNGDISN

SEQ ID NO: 56 - protein, 213 aa, Zea mays
MSSGSGSGNKRAAAERSAGAGAGSGDEDDDGAARKKLRLSKDQAAVLEECFKTHHTLTPKQKVALASSLGLRPRQVEVWFQNRRARTKLKQTEVDCEYLKRWCEQLAEENRRLGKEVAELRALSAAPAAPLTTLTMCLSCRRVASSSPSSSSSPRPSIPGAAAASGGSMASPAAAATLPAHRQFFCGFRDAGAAAAAYGTASAGLAKPVRAAR

SEQ ID NO: 57 - protein, 423 aa, synthetic construct
  MUTAGEN 299: note = C299A mutation
  source 1..423: note = corn putative glutamine synthetase (with C299A mutation)
MAQAVVPAMQCRVGVKAAAGRVWSAGRTRTGRGGASPGFKVMAVSTGSTGVVPRLEQLLNMDTTPYTDKVIAEYIWVGGSGIDIRSKSRTISKPVEDPSELPKWNYDGSSTGQAPGEDSEVILYPQAIFKDPFRGGNNVLVICDTYTPQGEPLPTNKRHRAAQIFSDPKVAEQVPWFGIEQEYTLLQKDVNWPLGWPVGGFPGPQGPYYCAVGADKSFGRDISDAHYKACLYAGINISGTNGEVMPGQWEYQVGPSVGIEAGDHIWISRYILERITEQAGVVLTLDPKPIQGDWNGAGAHTNYSTKTMREDGGFEEIKRAILNLSLRHDLHISAYGEGNERRLTGKHETASIGTFSWGVANRGCSIRVGRDTEAKGKGYLEDRRPASNMDPYIVTGLLAETTILWQPSLEAEALAAKKLALKV

SEQ ID NO: 58 - protein, 594 aa, Zea mays
MVKRSKKSKSKRVTLRQKHKVQRKVKEHHRKKRKEAKKAGKAGQRRKVEKDPGIPNEWPFKEQELKALEARRAQALQELELKKQARKERAQKRKAGLLEDEDIASLASAASAQGSEFAAKENAPLLVAKINDHSERSFYKELVKVIEASDVIMEVLDARDPLGTRCIDMEKMVRKADPSKRIVLLLNKIDLVPKEAAEKWLTYLREELPTVAFKCNTQEQRTKLGWKSSKLDKTSNIPQSSDCLGAENLIKLLKNYSRSHELKLTITVGIVGLPNVGKSSLINSLKRSRVVNVGSTPGITRSMQEVQLDKKVKLLDCPGVVMLKSSNSGVSVALRNCKKVEKIEDPVAPVKQILSICPHEKLLSLYKVPNFGSVDDFLQKVATVRGKLKKGGVVDVEAAARIVLHDWNEGKIPYFTLPPKRDAGEDSDAVIISEDGKEFNIDEIYKAESSYIGGLKSIEEFHHIEIPPNAPLAIDEEMLEDGGKKPSEAIQESRDREEQMPDVKDSGGSKAASASTQNDKLYTAEGVLDPRKSKAEKKRRKASRPSALNDMDADYDFKVDYRMEDGGSEGAHADDEDGGDGSEDNEPMTGVDDA

SEQ ID NO: 59 - protein, 423 aa, Zea mays
MAQAVVPAMQCRVGVKAAAGRVWSAGRTRTGRGGASPGFKVMAVSTGSTGVVPRLEQLLNMDTTPYTDKVIAEYIWVGGSGIDIRSKSRTISKPVEDPSELPKWNYDGSSTGQAPGEDSEVILYPQAIFKDPFRGGNNVLVICDTYTPQGEPLPTNKRHRAAQIFSDPKVAEQVPWFGIEQEYTLLQKDVNWPLGWPVGGFPGPQGPYYCAVGADKSFGRDISDAHYKACLYAGINISGTNGEVMPGQWEYQVGPSVGIEAGDHIWISRYILERITEQAGVVLTLDPKPIQGDWNGAGCHTNYSTKTMREDGGFEEIKRAILNLSLRHDLHISAYGEGNERRLTGKHETASIGTFSWGVANRGCSIRVGRDTEAKGKGYLEDRRPASNMDPYIVTGLLAETTILWQPSLEAEALAAKKLALKV

SEQ ID NO: 60 - protein, 971 aa, Zea mays
MIVQPIELRAWTAFPGSAQEGIGRMAASVSRAICVQKPGSKCTRDREATSFARRSVAAPRPPHAKAAGVIRSDSGAGRGQHCSPLRAVVDAAPIQTTKKRVFHFGKGKSEGNKTMKELLGGKGANLAEMASIGLSVPPGFTVSTEACQQYQDAGCALPAGLWAEIVDGLQWVEEYMGATLGDPQRPLLLSVRSGAAVSMPGMMDTVLNLGLNDEVAAGLAAKSGERFAYDSFRRFLDMFGNVVMDIPRSLFEEKLEHMKESKGLKNDTDLTASDLKELVGQYKEVYLSAKGEPFPSDPKKQLELAVLAVFNSWESPRAKKYRSINQITGLRGTAVSVQCMVFGNMGNTSDTGVLFTRNPNTGEKKLYGEFLVNAQGEDVVAGIRTPEDLDAMKNLMPQAYDELVENCNILESHYKEMQDIEFTVQENRLWMLQCRTGKRTGKSAVKIAVDMVNEGLVEPRSAIKMVEPGHLDQLLHPQFENPSTYKDQVIATGLPASPGAAVGQVVFTAEDAEAWHPQGKAAILVRAETSPEDVGGMHAAVGILTERGGMTSHAAVVARGWGKCCVSGCSGIRVNDAEKLVTIGGHVLREGEWLSLNGSAGEVILGKQPLSPPALSGDLGTFMAWVDDVRKLKVLANADTPDDALTARNNGAQGIGLCRTEHMFFASDERIKAVRQMIMAPTLELRQQALDRLLPYQRSDFEGIFRAMDGLPVTIRLLDPPLHEFLPEGNIEDIVSELCAETGANQEDALARIEKLSEVNPMLGFRGCRLGISYPELTEMQARAIFEAAIAMTNQGVQVFPEIMVPLVGTPQELGHQVTLIRQVAEKVFANVGKTIGYKVGTMIEIPRAALVADEIAEQAEFFSFGTNDLTQMTFGYSRDDVGKFIPVYLAQGILKHDPFEVLDQRGVGELVKFATERGRKARPNLKVGICGEHGGEPSSVAFFAKAGLDYVSCSPFRVPIARLAAAQVLV

SEQ ID NO: 61 - protein, 263 aa, Sorghum bicolor
MLELRLVQGSLLKKVLEAIRELVTDANFDCSGTGFSLQAMDSSHVALVALLLRAEGFEHYRCDRNLSMGMNLNNMAKMLRCAGNDDIITIKADDGSDTVTFMFESPKQDKIADFEMKLMDIDSEHLGIPDSEYQAIVRMPSAEFMRICKDLSSIGDTVVISVTKEGVKFSTSGEIGSANIVCRQNQTIDKPEEATIIEMQEPVSLTFALRYMNSFTKASSLSEQVTISLSSELPVVVEYKIAEMGYIRFYLAPKIEEDEEMKA

SEQ ID NO: 62 - protein, 447 aa, Escherichia coli
  source 1..447: note = Escherichia coli CFT073; note = strain = CFT073
MDQTYSLESFLNHVQKRDPNQTEFAQAVREVMTTLWPFLEQNPKYRQMSLLERLVEPERVIQFRVVWVDDRNQVQVNRAWRVQFSSAIGPYKGGMRFHPSVNLSILKFLGFEQTFKNALTTLPMGGGKGGSDFDPKGKSEGEVMRFCQALMTELYRHLGADTDVPAGDIGVGGREVGFMAGMMKKLSNNTACVFTGKGLSFGGSLIRPEATGYGLVYFTEAMLKRHGMGFEGMRVSVSGSGNVAQYAIEKAMEFGARVITASDSSGTVVDESGFTKEKLARLIEIKASRDGRVADYAKEFGLVYLEGQQPWSVPVDIALPCATQNELDVDAAHQLIANGVKAVAEGANMPTTIEATELFQQAGVLFAPGKAANAGGVATSGLEMAQNAARLGWKAEKVDARLHHIMLDIHHACVEHGGEGEQTNYVQGANIAGFVKVADAMLSQGVI

SEQ ID NO: 63 - protein, 594 aa, Zea mays
MVKRSKKSKSKRVTLRQKHKVQRKVKEHHRKKRKEAKKAGKAGQRRKVEKDPGIPNEWPFKEQELKALEARRAQALQELELKKQARKERAQKRKAGLLEDEDIASLASAASAQGSEFAAKENAPLLVAKINDHSERSFYKELVKVIEASDVIVEVLDARDPLGTRCIDMEKMVRKADPSKRIVLLLNKIDLVPKEAAEKWLTYLREELPTVAFKCNTQEQRTKLGWKSSKLDKTSNIPQSSDCLGAENLIKLLKNYSRSHELKLAITVGIVGLPNVGKSSLINSLKRSRVVNVGSTPGITRSMQEVQLDKKVKLLDCPGVVMLKSSNSGVSVALRNCKKVEKIEDPVAPVKEILSICPHEKLLSLYKVPNFGSVDDFLQKVATVRGKLKKGGVVDVEAAARIVLHDWNEGKIPYFTLPPKRDAGEDSDAVIISEDGKEFNIDDIYKAESSYIGGLKSIEEFHHIEIPPNAPPAIDEEMLEDGGKKPSEAIQESRDREEQMPDVKDSGGSKAASASTQNDKLYTAEGVLDPRKSKAEKKRRKASRPSALNDMDADYDFKVDYRMEDGGSEGAHADDEDGGDGSEDNEPMTGVDDA

SEQ ID NO: 64 - protein, 178 aa, Zea mays
MAEAPASPGGGGGSHESGSPRGGGGGGSVREQDRFLPIANISRIMKKAIPANGKIAKDAKETVQECVSEFISFITSEASDKCQREKRKTINGDDLLWAMATLGFEDYIEPLKVYLQKYREMEGDSKLTAKSSDGSIKKDALGHVGASSSAAEGMGQQGAYNQGMGYMQPQYHNGDISN

SEQ ID NO: 65 - protein, 447 aa, Escherichia coli
  source 1..447: note = strain = O157:H7 EDL933; note = Escherichia coli O157:H7 EDL933
MDQTYSLESFLNHVQKRDPNQTEFAQAVREVMTTLWPFLEQNPKYRQMSLLERLVEPERVIQFRVVWVDDRNQVQVNRAWRVQFSSAIGPYKGGMRFHPSVNLSILKFLGFEQTFKNALTTLPMGGGKGGSDFDPKGKSEGEVMRFCQALMTELYRHLGADTDVPAGDIGVGGREVGFMAGMMKKLSNNTACVFTGKGLSFGGSLIRPEATGYGLVYFTEAMLKRHGMGFEGMRVSVSGSGNVAQYAIEKAMEFGARVITASDSSGTVVDESGFTKEKLARLIEIKSSRDGRVADYAKEFGLVYLEGQQPWSVPVDIALPCATQNELDVDAAHQLIANGVKAVAEGANMPTTIEATELFQQAGVLFAPGKAANAGGVATSGLEMAQNAARLGWKAEKVDARLHHIMLDIHHACVEHGGEGEQTNYVQGANIAGFVKVADAMLAQGVI

SEQ ID NO: 66 - protein, 447 aa, Escherichia coli
MDQTYSLESFLNHVQKRDPNQTEFAQAVREVMTTLWPFLEQNPKYRQMSLLERLVEPERVIQFRVVWVDDRNQIQVNRAWRVQFSSAIGPYKGGMRFHPSVNLSILKFLGFEQTFKNALTTLPMGGGKGGSDFDPKGKSEGEVMRFCQALMTELYRHLGADTDVPAGDIGVGGREVGFMAGMMKKLSNNTACVFTGKGLSFGGSLIRPEATGYGLVYFTEAMLKRHGMGFEGMRVSVSGSGNVAQYAIEKAMEFGARVITASDSSGTVVDESGFTKEKLARLIEIKASRDGRVADYAKEFGLVYLEGQQPWSLPVDIALPCATQNELDVDAAHQLIANGVKAVAEGANMPTTIEATELFQQAGVLFAPGKAANAGGVATSGLEMPQNAARLGWKAEKVDARLHHIMLDIHHACVEHGGEGEQTNYVQGANIAGFVKVADAMLAQGVI

SEQ ID NO: 67 - protein, 947 aa, Zea mays
MAASVSRAICVQKPGSKCTRDREATSFARRSVAAPRPPHAKAAGVIRSDSGAGRGQHCSPLRAVVDAAPIQTTKKRVFHFGKGKSEGNKTMKELLGGKGANLAEMASIGLSVPPGFTVSTEACQQYQDAGCALPAGLWAEIVDGLQWVEEYMGATLGDPQRPLLLSVRSGAAVSMPGMMDTVLNLGLNDEVAAGLAAKSGERFAYDSFRRFLDMFGNVVMDIPRSLFEEKLEHMKESKGLKNDTDLTASDLKELVGQYKEVYLSAKGEPFPSDPKKQLELAVLAVFNSWESPRAKKYRSINQITGLRGTAVNVQCMVFGNMGNTSGTGVLFTRNPNTGEKKLYGEFLVNAQGEDVVAGIRTPEDLDAMKNLMPQAYDELVENCNILESHYKEMQDIEFTVQENRLWMLQCRTGKRTGKSAVKIAVDMVNEGLVEPRSAIKMVEPGHLDQLLHPQFENPSAYKDQVIATGLPASPGAAVGQVVFTAEDAEAWHSQGKAAILVRAETSPEDVGGMHAAVGILTERGGMTSHAAVVARWWGKCCVSGCSGIRVNDAEKLVTIGSHVLREGEWLSLNGSTGEVILGKQPLSPPALSGDLGTFMAWVDDVRKLKVLANADTPDDALTARNNGAQGIGLCRTEHMFFASDERIKAVRQMIMAPTLELRQQALDRLLTYQRSDFEGIFRAMDGLPVTIRLLDHPSYEFLPEGNIEDIVSELCAETGANQEDALARIEKLSEVNPMLGFRGCRLGISYPELTEMQARAIFEAAIAMTNQGVQVFPEIMVPLVGTPQELGHQVTLIRQVAEKVFANVGKTIGYKVGTMIEIPRAALVADEIAEQAEFFSFGTNDLTQMTFGYSRDDVGKFIPVHLAQGILQHDPFEVLDQRGVGELVKFATERGRKARPNLKVGICGEHGGEPSSVAFFAKAGLDFVSCSPFRVPIARLAAAQVLV

SEQ ID NO: 68 - protein, 179 aa, Zea mays
MAEAPASPGGGGGSHESGSPRGGGGGGSVREQDRFLPIANISRIMKKAIPANGKIAKDAKETVQECVSEFISFITSEASDKCQREKRKTINGDDLLWAMATLGFEDYIEPLKVYLQKYREMEGDSKLTAKSSDGSIKKDALGHVGASSSAAEGMGQQGAYNQGMGYMQPQYHNGDISNV

SEQ ID NO: 69 - protein, 447 aa, Escherichia coli
  source 1..447: note = strain = 042; note = Escherichia coli 042
MDQTYSLESFLNHVQKRDPNQTEFAQAVREVMTTLWPFLEQNPKYRQMSLLERLVEPERVIQFRVVWVDDRNQVQVNRAWRVQFSSAIGPYKGGMRFHPSVNLSILKFLGFEQTFKNALTTLPMGGGKGGSDFDPKGKSEGEVMRFCQALMTELYRHLGADTDVPAGDIGVGGREVGFMAGMMKKLSNNTACVFTGKGLSFGGSLIRPEATGYGLVYFTEAMLKRHGMGFEGMRVSVSGSGNVAQYAIEKAMEFGARVITASDSSGTVVDESGFTKEKLARLIEIKASRDGRVADYAKEFGLVYLEGQQPWSVPVDIALPCATQNELDVDAAHQLIATGVKAVAEGANMPTTIEATELFQQAGVLFAPGKAANAGGVATSGLEMAQNAARLGWKAEKVDARLHHIMLDIHHACVEHGGEGEQTNYVQGANIAGFVKVADAMLAQGVI

SEQ ID NO: 70 - protein, 345 aa, Glycine max
MCGGAIISDFIPAAAIAGSRRLTADYLWPDLKKRKSDLDVDFEADFRDFKDDSDIDDDDDDHQVKPFAFAASSRLSTAAKSVAFQGRAEISANRKRKNQYRGIRQRPWGKWAAEIRDPRKGVRVWLGTFNTAEEAARAYDAEARRIRGKKAKVNFPEAPGTSSVKRSKVNPQENLKTVQPNLGHKFSAGNNHMDLVEQKPLVSQYANMASFPGSGNGLRSLPSSDDATLYFSSDQGSNSFDYAPEISSMLSAPLDCESHFVQNANQQQPNSQNVVSIEDDSAKTLSEELVDIESELKFFQMPYLEGSWGDTSLESLLSGDTTQDGGNLMNLWCLDDIPSMAGGVF

SEQ ID NO: 71 - protein, 185 aa, Zea mays
MAEAPASPGGGGGSHESGSPRGGGGGGSVREQDRFLPIANISRIMKKAIPANGKTIPANGKIAKDAKETVQECVSEFISFITSEASDKCQREKRKTINGDDLLWAMATLGFEDYIEPLKVYLQKYREMEGDSKLTAKSSDGSIKKDALGHVGASSSAAQGMGQQGAYNQGMGYMQPQYHNGDISN

SEQ ID NO: 72 - protein, 355 aa, Arabidopsis thaliana
IYHRKVVFTYVRAKRFYHFLNIEMVTDFKSLLPVIDISPLLAKCDDFDMAEDAGVVEVVGKLDRACRDVGFFYVIGHGISDDLINKVKEMTHQFFELPYEEKLKIKITPTAGYRGYQRIGVNFTSGKQDMHEAIDCYREFKQGKHGDIGKVLEGPNQWPGNPQEYKDLMEKYIKLCTDLSRNILRGISLALGGSPYEFEGKMLRDPFWVMRIIGYPGVNQENVIGCGAHTDYGLLTLINQDDDKTALQVKNVDGDWIPAIPIPGSFICNIGDMLTILSNGVYQSTLHKVINNSPKYRVCVAFFYETNFEAEVEPLDIFKEKHPRKETSQVAKRVVYGQHLINKVLTTFANLVENS

SEQ ID NO: 73 - protein, 131 aa, Arabidopsis thaliana
MDQGGRSSGSGGGGAEQGKYRGVRRRPWGKYAAEIRDSRKHGERVWLGTFDTAEDAARAYDRAAYSMRGKAAILNFPHENNMGTGSSSTAANSSSSSQQVFEFEYLDDSVLDELLEYGENYNKTHNINMGK

SEQ ID NO: 74 - protein, 286 aa, Oryza sativa
  source 1..286: note = Oryza sativa Japonica Group
MGMANEESPNYQVKKGGRIPPRSSLIYPFMSMGPAAGEGCGLCGADGGGCCSRHRHDDDGFPFVFPPSACQGIGAPAPPVHEFQFFGNDGGGDDGESVAWLFDDYPPPSPVAAAAGMHHRQPPYDGVVAPPSLFRRNTGAGGLTFDVSLGERPDLDAGLGLGGGGGRHAEAAASATIMSYCGSTFTDAASSMPKEMVAAMADDGESLNPNTVVGAMVEREAKLMRYKEKRKKRCYEKQIRYASRKAYAEMRPRVRGRFAKEPDQEAVAPPSTYVDPSRLELGQWFR

SEQ ID NO: 75 - protein, 272 aa, Zea mays
MAPQSLDLGLSLGLGVAAFQPSSFCHPGNAVVVPAAAEREASPAAAEERERRCSPAGSPVSSGSGSGNKRAAAERSAGAGAGSGDEDDDGAARKKLRLSKDQAAVLEECFKTHHTLTPKQKVALASSLGLRPRQVEVWFQNRRARTKLKQTEVDCEYLKRWCEQLAEENRRLGKEVAELRALSAAPAAPLTTLTMCLSCRRVASSSPSSSSSPRPSIPGAAAASGGSMASPAAAATLPAHRQFFCGFRDAGAAAAAYGTASAGLAKPVRAAR

SEQ ID NO: 76 - protein, 594 aa, Zea mays
MVKRSKKSKSKRVTLRQKHKVQRKVKEHHRKKRKEAKKAGKAGQRRKVEKDPGIPNEWPFKEQELKALEARRAQALQELELKKQARKERAQKRKAGLLEDEDIASLASAASAQGSEFAAKENAPLLVAKINDHSERSFYKELVKVIEASDVIMEVLDARDPLGTRCIDMEKMVRKADPSKRIVLLLNKIDLVPKEAVEKWLTYLREELPTVAFKCNTQEQRTKLGWKSSKLDKTSNIPQSSDCLGAENLIKLLKNYSRSHELKLAITVGIVGLPNVGKSSLINSLKRSRVVNVGSTPGITRSMQEVQLDKKVKLLDCPGVVMLKSSNSGVSVALRNCKRVEKIEDPVAPVKEILSICPHEKLLSLYKVPNFGSVDDFLQKVATVRGKLKKGGVVDVEAAARIVLHDWNEGKIPYFTLPPKRDAGEDSDAVIISEDGKEFNIDEIYKAESSYIGGLKSIEEFHHIEIPPNAPLAIDEEMLEDGGKKPSEAIQESRDREEQMPDVKDSGGSKAASASTQNDKLYTADAILDPHKRKAEKKRRKASRPSALNDMDADYDFKVDYRMEDGGSEGAHADDEDGGDGSEDNEPMTGVDDA

SEQ ID NO: 77 - protein, 133 aa, Arabidopsis thaliana
MDQGGRSSGSGGGGAEQGKYRGVRRRPWGKYAAEIRDSRKHGERVWLGTFDTAEDAARAYDRAAYSMRGKAAILNFPHEYNMGTGSSSTAANSSSSSQQVFEFEYLDDSVLDELLEYGENYNKTHNINMGKRQ

SEQ ID NO: 78 - protein, 497 aa, Arabidopsis thaliana
MIIYILLLLNRTSHHRDSASNSSNHKCQQQKPRKDKQKQVEQNTKKIEEHQIKSESTLLISNHNVNMSSQSNNSESTSTNNSSKPHTGGDIRWDAVNSLKSRGIKLGISDFRVLKRLGYGDIGSVYLVELKGANPTTYFAMKVMDKASLVSRNKLLRAQTEREILSQLDHPFLPTLYSHFETDKFYCLVMEFCSGGNLYSLRQKQPNKCFTEDAARFFASEVLLALEYLHMLGIVYRDLKPENVLVRDDGHIMLSDFDLSLRCSVNPTLVKSFNGGGTTGIIDDNAAVQGCYQPSAFFPRMLQSSKKNRKSKSDFDGSLPELMAEPTNVKSMSFVGTHEYLAPEIIKNEGHGSAVDWWTFGIFIYELLHGATPFKGQGNKATLYNVIGQPLRFPEYSQVSSTAKDLIKGLLVKEPQNRIAYKRGATEIKQHPFFEGVNWALIRGETPPHLPEPVDFSCYVKKEKESLPPAATEKKSKMFDEANKSGSDPDYIVFEYF

SEQ ID NO: 79 - protein, 131 aa, Arabidopsis thaliana
MDQGGRSSGSGGGGAEQGKYRGVRRRPWGKYAAEIRDSRKHGERVWLGTFDTAEDAARAYDRAAYSMRGKAAILNFPHEYNMGTGSSSTAANSSSSSQQVFEFEYLDDSVLDELLEYGENYNKTHNINVGK

SEQ ID NO: 80 - protein, 690 aa, Arabidopsis thaliana
MDATEQSLRQSLSEKSSSVEAQGNAVRALKASRAAKPEIDAAIEQLNKLKLEKSTVEKELQSIISSSGNGSLNREAFRKAVVNTLERRLFYIPSFKIYSGVAGLFDYGPPGCAIKSNVLSFWRQHFILEENMLEVDCPCVTPEVVLKASGHVDKFTDLMVKDEKTGTCYRADHLLKDYCTEKLEKDLTISAEKAAELKDVLAVMEDFSPEQLGAKIREYGITAPDTKNPLSDPYPFNLMFQTSIGPSGLIPGYMRPETAQGIFVNFKDLYYYNGKKLPFAAAQIGQAFRNEISPRQGLLRVREFTLAEIEHFVDPENKSHPKFSDVAKLEFLMFPREEQMSGQSAKKLCLGEAVAKGTVNNETLGYFIGRVYLFLTRLGIDKERLRFRQHLANEMAHYAADCWDAEIESSYGWIECVGIADRSAYDLRAHSDKSGTPLVAEEKFAEPKEVEKLVITPVKKELGLAFKGNQKNVVESLEAMNEEEAMEMKATLESKGEVEFYVCTLKKSVNIKKNMVSISKEKKKEHQRVFTPSVIEPSFGIGRIIYCLYEHCFSTRPSKAGDEQLNLFRFPPLVAPIKCTVFPLVQNQQFEEAAKVISKELASVGISHKIDITGTSIGKRYARTDELGVPFAITVDSDTSVTIRERDSKDQVRVTLKEAASVVSSVSEGKMTWQDVWATFPHHSSAAADE

SEQ ID NO: 81 - protein, 460 aa, Arabidopsis thaliana
MTVVGDVAPIPRRNSSTCSNDIAAPLLPECHGDEVAHDEFNGASFSGAVFNLATTIIGAGIMALPATMKILGLGLGITMIVVMAFLTDASIEFLLRFSKAGKNRSYGGLMGGSFGNPGRILLQVAVLVNNIGVLIVYMIIIGDVLAGKTEDGIHHFGVLEGWFGHHWWNGRAAILLITTLGVFAPLACFKRIDSLKFTSALSVALAVVFLIITAGISIMKLISGGVAMPRLLPDVTDLTSFWNLFTVVPVLVTAFICHYNVHSIQNELEDPSQIRPVVRSALMLCSSVYIMTSIFGFLLFGDDTLDDVLANFDTDLGIPFGSILNDAVRVSYALHLMLVFPIVFCPLRINIDGLLFPSARSLSTSNVRFGCLTAGLISVIFLGANFIPSIWDAFQFTGATAAVCLGFIFPASIILKDRHDKATNRDTTLAIFMIVLAVLSNAIAIYSDAYALFKKNAPRE

SEQ ID NO: 82 - protein, 180 aa, Zea mays
MADAPASPGGGGGSHESGSPRGGGGGGGGSVREQDRFLPIANISRIMKKAIPANGKIAKDAKETVQECVSEFISFITSEASDKCQREKRKTINGDDLLWAMATLGFEDYIEPLKVYLQKYREMEGDSKLTSKSSDGSIKKDALGHVGASSSAVQGMGQQGTYNQGMGYMQPQYHNGDISN

SEQ ID NO: 83 - protein, 971 aa, Zea mays
MIVQPIELRAWTAFPGSAQEGIGRMAASVSRAICVQKPGSKCTRDREATSFARRSVAAPRPPHAKAAGVIRSDSGAGRGQHCSPLRAVVDAAPIQTTKKRVFHFGKGKSEGNKTMKELLGGKGANLAEMASIGLSVPPGFTVSTEACQQYQDAGCALPAGLWAEIVDGLQWVEEYMGATLGDPQRPLLLSVRSGAAVSMPGMMDTVLNLGLNDEVAAGLAAKSGERFAYDSFRRFLDMFGNVVMDIPRSLFEEKLEHMKESKGLKNDTDLTASDLKELVGQYKEVYLSAKGEPFPSDPKKQLELAVLAVFNSWESPRAKKYRSINQITGLRGTAVNVQCMVFGNMGNTSGTGVLFTRNPNTGEKKLYGEFLVNAQGEDVVAGIRTPEDLDAMKNLMPQAYDELVENCNILESHYKEMQDIEFTVQENRLWMLQCRTGKRTGKSAVKIAVDMVNEGLVEPRSAIKMVEPGHLDQLLHPQFENPSAYKDQVIATGLPASPGAAVGQVVFTAEDAEAWHSQGKAAILVRAETSPEDVGGMHAAVGILTERGGMTSHAAVVARGWGKCCVSGCSGIRVNDAEKLVTIGGHVLREGEWLSLNGSTGEVILGKQPLSPPALSGDLGTFMAWVDDVRKLKVLANADTPDDALTARNNGAQGIGLCRTEHMFFASDERIKAVRQMIMAPTLELRQQALDRLLPYQRSDFEGIFRAMDGLPVTIRLLDPPLHEFLPEGNIEDIVSELCAETGANQEDALARIEKLSEVNPMLGFRGCRLGISYPELTEMQARAIFEAAIAMTNQGVQVFPEIMVPLVGTPQELGHQVTLIRQVAEKVFANVGKTIGYKVGTMIEIPRAALVADEIAEQAEFFSFGTNDLTQMTFGYSRDDVGKFIPVYLAQGILQHDPFEVLDQRGVGELVKFATERGRKARPNLKVGICGEHGGEPSSVAFFAKAGLDYVSCSPFRVPIARLAAAQVLV

SEQ ID NO: 84 - protein, 523 aa, Chlorella sorokiniana
CRPPSSPSLSWPPGLRSPALPRAVACARGRSAKRDVAAKRLRSRSPRMDATTGDFTALQKAVKQMATKAGTEGLVHGIKNPELRQLLTEIFMKDPEQQEFMQAVREVAVSLQPVFEKRPELLPIFKQIVEPERVITFRVSWLDDAGNLQVNRGFRVQYSSAIGPYKGGLRFHPSVNLSIMKFLAFEQIFKNSLTTLPMGGGKGGSDFDPKGKSDAEVMRLWQSFMTELQRHISYVQDVPAGDIGVGAREIGYLFGQYKRITKNYTGVLTGKGQEYGGSEIRPEATGYGAVLFVENVLKDKGESLKGKRCLVSGAGNVAQYCAELLLEKGAIVLSLSDSQGYVYEPNGFTREQLQAVQDMKKKNNSARISEYKSDTAVYVGDRRKPWELDCQVDIAFPCATQNEIDEHDAELLIKHGCQYVVEGANMPSTNEAIHKYNKAGIIYCPGKAANAGGVAVSGLEMTQNRMSLNWTREEVRDKLERIMKDIYDSAMGPSREYNVDLAAGANIAGFTKVADAVKAQGAV

SEQ ID NO: 85 - protein, 947 aa, Zea mays
MTASVSRAICVQKPGSKCTRDREATSFARRSVAAPRPPHAKAAGVIRSDSGAGRGQHCSPLRAVVDAAPIQTTKKRVFHFGKGKSEGNKTMKELLGGKGANLAEMASIGLSVPPGFTVSTEACQQYQDAGCALPAGLWAEIVDGLQWVEEYMGATLGDPQRPLLLSVRSGAAVSMPGMMDTVLNLGLNDEVAAGLAAKSGERFAYDSFRRFLDMFGNVVMDIPRSLFEEKLEHMKESKGLKNDTDLTASDLKELVGQYKEVYLSAKGEPFPSDPKKQLELAVLAVFNSWESPRAKKYRSINQITGLRGTAVNVQCMVFGNMGNTSGTGVLFTRNPNTGEKKLYGEFLVNAQGEDVVAGIRTPEDLDAMKNLMPQAYDELVENCNILESHYKEMQDIEFTVQENRLWMLQCRTGKRTGKSAVKIAVDMVNEGLVEPRSAIKMVEPGHLDQLLHPQFENPSAYKDQVIATGLPASPGAAVGQVVFTAEDAEAWHSQGKAAILVRAETSPEDVGGMHAAVGILTERGGMTSHAAVVARGWGKCCVSGCSGIRVNDAEKLVTIGGHVLREGEWLSLNGSTGEVILGKQPLSPPALSGDLGTFMAWVDDVRKLKVLANADTPDDALTARNNGAQGIGLCRTEHMFFASDERIKAVRQMIMAPTLELRQQALDRLLPYQRSDFEGIFRAMDGLPVTIRLLDPPLHEFLPEGNIEDIVSELCAETGANQEDALARIEKLSEVNPMLGFRGCRLGISYPELTEMQARAIFEAAIAMTNQGVQVFPEIMVPLVGTPQELGHQVTLIRQVAEKVFANVGKTIGYKVGTMIEIPRAALVADEIAEQAEFFSFGTNDLTQMTFGYSRDDVGKFIPVYLAQGILQHDPFEVLDQRGVGELVKLATERGRKARPNLKVGICGEHGGEPSSVAFFAKAGLDYVSCSPFRVPIARLAAAQVLV

SEQ ID NO: 86 - protein, 947 aa, Zea mays
MAASVSRAICVQKPGSKCTRDREATSFARRSVAAPRPPHAKARRRHPLRLRRGTGPHCSPLRAVVDAAPIQTTKKRVFHFGKGKSEGNKTMKELLGGKGANLAEMASIGLSVPPGFTVSTEACQQYQDAGCALPAGLWAEIVDGLQWVEEYMGATLGDPQRPLLLSVRSGAAVSMPGMMDTVLNLGLNDEVAAGLAAKSGERFAYDSFRRFLDMFGNVVMDIPRSLFEEKLEHMKESKGLKNDTDLTASDLKELVGQYKEVYLSAKGEPFPSDPKKQLELAVLAVFNSWESPRAKKYRSINQITGLRGTAVNVQCMVFGNMGNTSGTGVLFTRNPNTGEKKLYGEFLVNAQGEDVVAGIRTPEDLDAMKNLMPQAYDELVENCNILESHYKEMQDIEFTVQENRLWMLQCRTGKRTGKSAVKIAVDMVNEGLVEPRSAIKMVEPGHLDQLLHPQFENPSAYKDQVIATGLPASPGAAVGQVVFTAEDAEAWHSQGKAAILVRAETSPEDVGGMHAAVGILTERGGMTSHAAVVARWWGKCCVSGCSGIRVNDAEKLVTIGSHVLREGEWLSLNGSTGEVILGKQPLSPPALSGDLGTFMAWVDDVRKLKVLANADTPDDALTARNNGAQGIGLCRTEHMFFASDERIKAVRQMIMAPTLELRQQALDRLLTYQRSDFEGIFRAMDGLPVTIRLLDHPSYEFLPEGNIEDIVSELCAETGANQEDALARIEKLSEVNPMLGFRGCRLGISYPELTEMQARAIFEAAIAMTNQGVQVFPEIMVPLVGTPQELGHQVTLIRQVAEKVFANVGKTIGYKVGTMIEIPRAALVADEIAEQAEFFSFGTNDLTQMTFGYSRDDVGKFIPVHLAQGILQHDPFEVLDQRGVGELVKFATERGRKARPNLKVGICGEHGGEPSSVAFFAKAGLDFVSCSPFRVPIARLAAAQVLV

SEQ ID NO: 87 - protein, 282 aa, Arabidopsis thaliana
MGTRALQIPLLEGETDNYDGVTVTMVEPMDSEVFTESLRASLSHWREEGKKGIWIKLPLGLANLVEAAVSEGFRYHHAEPEYLMLVSWISETPDTIPANASHVVGAGALVINKNTKEVLVVQERSGFFKDKNVWKLPTGVINEGEDIWTGVAREVEEETGIIADFVEVLAFRQSHKAILKKKTDMFFLCVLSPRSYDITEQKSEILQAKWMPIQEYIDQPWNKKNEMFKFMANICQKKCEEEYLGFAIVPTTTSSGKESFIYCNADHAKRLKVSRDQASASL

SEQ ID NO: 88 - protein, 567 aa, Arabidopsis thaliana
IVLVVAVLTHTASAAVREYHWEVEYKYWSPDCKEGAVMTVNGEFPGPTIKAFAGDTIVVNLTNKLTTEGLVIHWHGIRQFGSPWADGAAGVTQCAINPGETFTYNFTVEKPGTHFYHGHYGMQRSAGLYGSLIVDVAKGKSERLRYDGEFNLLLSDWWHEAIPSQELGLSSKPMRWIGEAQSILINGRGQFNCSLAAQFSNNTSLPMCTFKEGDQCAPQILHVEPNKTYRIRLSSTTALASLNLAVQGHKLVVVEADGNYITPFTTDDIDIYSGESYSVLLTTDQDPSHNYYISVGVRGRKPNTTQALTILNYVTAPASKLPSSPPPVTPRWDDFERSKNFSKKIFSAMGSPSPPKKYRKRLILLNTQNLIDGYTKWAINNVSLVTPATPYLGSVKYNLKLGFNRKSPPRSYRMDYDIMNPPPFPNTTTGNGIYVFPFNVTVDVIIQNANVLKGIVSEIHPWHLHGHDFWVLGYGDGKFKPGIDEKTYNLKNPPLRNTAILYPYGWTAIRFVTDNPGVWFFHCHIEPHLHMGMGVVFAEGLNRIGKVPDEALGCGLTKQFLMNRNRN

SEQ ID NO: 89 - protein, 523 aa, Chlorella sorokiniana
CRPPSSPSLSWPPGLRSPALPRAVACARGRSAKRDVAAKRLRSRSPRMDATTGDFTALQKAVKQMATKAGTEGLVHGIKNPELRQLLTEIFMKDPEQQEFMQAVREVAVSLQPVFEKRPELLPIFKQIVEPERVITFRVSWLDDAGNLQVNRGFRVQYSSAIGPYKGGLRFHPSVNLSIMKFLAFEQIFKNSLTTLPMGGGKGGSDFDPKGKSDAEVMRFCQSFMTELQRHISYVQDVPAGDIGVGAREIGYLFGQYKRITKNYTGVLTGKGQEYGGSEIRPEATGYGAVLFVENVLKDKGESLKGKRCLVSGAGNVAQYCAELLLEKGAIVLSLSDSQGYVYEPNGFTREQLQAVQDMKKKNNSARISEYKSDTAVYVGDRRKPWELDCQVDIAFPCATQNEIDEHDAELLIKHGCQYVVEGANMPSTNEAIHKYNKAGIIYCPGKAANAGGVAVSGLEMTQNRMSLNWTREEVRDKLERIMKDIYDSAMGPSREYNVDLAAGANIAGFTKVADAVKAQGAV

SEQ ID NO: 90 - protein, 131 aa, Arabidopsis thaliana
MDQGGRSSGSGGGGAEQGKYRGVRRRPWGKYAAEIRDSRKHGERVWLGTFDTAEDAARAYDRAAYSMRGKAAILNFPHEYNMGTGSSSTAANSSSSSQQVFEFEYLDDSVLDELLEYGENYNKTHNINMGK

SEQ ID NO: 91 - protein, 423 aa, Zea mays
MAQAVVPAMQCRVGVKAAAGRVWSAGRTRTGRGGASPGFKVMAVSTGSTGVVPRLEQLLNMDTTPYTDKVIAEYIWVGGSGIDIRSKSRTISKPVEDPSELPKWNYDGSSTGQAPGEDSEVILYPQAIFKDPFRGGNNVLVICDTYTPQGEPLPTNKRHRAAQIFSDPKVGEQVPWFGIEQEYTLLQKDVNWPLGWPVGGFPGPQGPYYCAVGADKSFGRDISDAHYKACLYAGINISGTNGEVMPGQWEYQVGPSVGIEAGDHIWISRYILERITEQAGVVLTLDPKPIQGDWNGAGCHTNYSTKTMREDGGFEEIKRAILNLSLRHDLHISAYGEGNERRLTGKHETASIGTFSWGVANRGCSIRVGRDTEAKGKGYLEDRRPASNMDPYIVTGLLAETTILWQPSLEAEALAAKKLALKV

SEQ ID NO: 92 - protein, 263 aa, Zea mays
MLELRLVQGSLLKKVLEAIRELVNDANFDCSGTGFSLQAMDSSHVALVALLLRAEGFEHYRCDRNLSMGMNLNNMAKMLRCAGNDDIITIKADDGSDTVTFMFESPKQDKIADFEMKLMDIDSEHLGIPDSEYQAIVRMPSSEFMRICKDLSSIGDTVVISVTKEGVKFSTSGEIGSANIVCRQNQTIDKPEEATIIEMQEPVSLTFALRYMNSFTKASSLSEQVTISLSSELPVVVEYKIAEMGYIRFYLAPKIEDDEEMKP

SEQ ID NO: 93 - DNA, 1388 bp, synthetic construct
  source 1..1388: mol_type = other DNA; note = seed preferred promoter
atcaacaaattactcctcaatcacactcctatagaaaacggtttaagctatcattacatgtctagttggttttactcagccctagaagtgttgtttattgcatcactttccacgaagcacaatttttcttttttacaatcaccagacctcacaggctcacacatatgctttagagcacattctaaactttgaactataaaagctgttaacactaatacactatgcgttcttttttgctccaaacacttttgatccattattaggagacactccacttagaaagattttctaatcctttggtcaactaggaagttcaaggtttttctaaacagaaattcatttcacaagtaatttaatttataaggaaatgaatagagaaatcaaatcattgaagaactacaaaatatagattcaaggtcaggtctaagaaaatattcctgaagctcaaaaaagagttttcctctcacattatagaattggcctttacttcaacattttcccacctattccacatttggtcagaacatttttaattacttgtggatcaatttccggttgaaatgggtttggtgaatatccggttcagttatatggtggccgttggaattggcttattagttgtggccgttgttgaagccgttggtattggtaagggagaagcagacttgtggctatgagtctatgaccatgactcgtgattatggagctgtcttatgaccctgaccatcaccttgatctggtggattccaatgttttcttcttcttctaataaaatattatggtcaatacaggtgctaattaagatggtaataatttcttatgtttctgtggtaaagtttgattcaattccgtagttttagataatcttatttccatacataaattttatagttttatctactttgttcttatgttttatctctagccaagagttattattattatcagaagaagaaaaaaaaaagaagcatatatacaaaaggtttaataaaatgtattatacaaggcaattatccaaattttttttgttttggtttacattgatgctctcaggatttcataaggatagagagatctattcgtatacgtgtcacgtcatgagtgggtgtttcgccaatccatgaaacgcacctagatatctaaaacacatatcaattgcgaatctgcgaagtgcgagccattaaccacgtaagcaaacaaacaatctaaaccccaaaaaaaatctatgactagccaatagcaacctcagagattgatatttcaagataagacagtatttagatttctgtattatatatagcgaaaatcgcatcaataccaaaccacccatttcttggcttacaacaacaaatcttaaacgttttactttgtgctgcactactcaacct

SEQ ID NO: 94 - DNA, 1785 bp, synthetic construct
  source 1..1785: mol_type = other DNA; note = root preferred promoter
taggaaaaaagtttatattcctacccaaactttcggcaccagagacaaattaggtttgttcagaaaatcagtcgctcatcgacagacttaacaccacaaaatcatcagaatttctttgggacaaaatggaaaatgttcttcacggctccacctaccaaaatttcgatatcaagctcaaaagcatacacaaactataactaattccatagttacgtaatcttaacttcgaattcaacaacacatgcatgcatcgactgaatcttcaacaaatgcaatcaaacacacaaaattgctaccaaaaaaatatgattttttttttatgatttcaattttcatccggcacttagtccaaaactttttttgtgtgtcaacattttttaaaaaaatctttaaacggatatctgatctaagagcatgttcataggtgatacttacaaaatatttttaagaaattttttagtattatttataatgtttgtttaataaatatatataagattttttgcttttatcaaatgtgaccaatcagaagaaaccacgtcagatgatactgatatgacaaatatgatactgatcaaacatattctaattgctttactaatataaaaataatttttggacttgtgatactctaaaaatatcacccatatacatggtctaatatatggatcgtaaaaaactcatatataatattaataagtagtagaagagcgtagaccatgtcctgggtcgtcgtccaaatgaccacaagaagatttcaaaacagaggaaaatatttctcattaaataagttttcctgacgcataagataacattattacaagattcagaaaaagaaaggtgaaaggataatgtttctcctactatataagatgtgtacatctgaaaaaatatgaatatatttgtaacgtttgactgttattacatgattaatacgatataaatattaacattttttttcaaaataaaagtaatatagtaaggaaatgaaaagaggcatgaagcatgcctctttttttggtcggctgccgtttacaattgccaattgcgatagttactcttcttgcgtgtacgacttttgtttttttttacatattcgccaataatttgacgttttctattagtttgtttgatactctgttgtcttgctaaaactcaataaaacattaaattactttcttgaatgaagctggaacaaatctaacataaatagaaaatgatgggcaagttgatgttattcgtaaatttatttagattatattatataaaaagcaatccaattatatatctcatatatacaatttcttatcttactttgtcaatgtcatatacgtaactaaaacttgcggaaatagaaaatgccacgtgtatggtggacataatccgaatctctctctttcttctataaatagtggccattcccattggttgaaatcacaaaagcatcataagaagaagaagaaactacaatagttaatcaatcaaagagaagtaagagaacaggtaaacccctagattctctcttctttacatttatatgcatatatgtagactatgtagttcgagcttcatggtacaaaattcaataaactcttcttatgacttcgtttacaattgtgtttgtgaatagacacaaaagattagttttgtttactttagattcaaaacacttcgggcctatcctgtatatatgctgatcagatgcatgtggttgattaatctttaatctcatcatattaattactaattttttgtttgtttgattaatttgtgtgtggtaaaggt

SEQ ID NO: 95 - DNA, 1243 bp, synthetic construct
  source 1..1243: mol_type = other DNA; note = meristem and endosperm preferred promoter
aaaaccaccgagggacctgatctgcaccggttttgatagttgagggacccgttgtgtctggttttccgatcgagggacgaaaatcggattcggtgtaaagttaagggacctcagatgaacttattccggagcatgattgggaagggaggacataaggcccatgtcgcatgtgtttggacggtccagatctccagatcactcagcaggatcggccgcgttcgcgtagcacccgcggtttgattcggcttcccgcaaggcggcggccggtggccgtgccgccgtagcttccgccggaagcgagcacgccgccgccgccgacccggctctgcgtttgcaccgccttgcacgcgatacatcgggatagatagctactactctctccgtttcacaatgtaaatcattctactattttccacattcatattgatgttaatgaatatagacatatatatctatttagattcattaacatcaatatgaatgtaggaaatgctagaatgacttacattgtgaattgtgaaatggacgaagtacctacgatggatggatgcaggatcatgaaagaattaatgcaagatcgtatctgccgcatgcaaaatcttactaattgcgctgcatatatgcatgacagcctgcatgcgggcgtgtaagcgtgttcatccattaggaagtaaccttgtcattacttataccagtactacatactatatagtattgatttcatgagcaaatctacaaaactggaaagcaataaggaatacgggactggaaaagactcaacattaatcaccaaatatttcgccttctccagcagaatatatatctctccatcttgatcactgtacacactgacagtgtacgcataaacgcagcagccagcttaactgtcgtctcaccgtcgcacactggccttccatctcaggctagctttctcagccacccatcgtacatgtcaactcggcgcgcgcacaggcacaaattacgtacaaaacgcatgaccaaatcaaaaccaccggagaagaatcgctcccgcgcgcggcggcggcgcgcacgtacgaatgcacgcacgcacgcccaaccccacgacacgatcgcgcgcgacgccggcgacaccggccatccacccgcgccctcacctcgccgactataaatacgtaggcatctgcttgatcttgtcatccatctcaccaccaaaaaaaaaggaaaaaaaaacaaaacacaccaagccaaataaaagcgacaa

SEQ ID NO: 96 - DNA, 2439 bp, synthetic construct
  source 1..2439: mol_type = other DNA; note = root specific promoter
ggaagctaactagtcacggcgaatacatgacgacatcggcctacaacgcacaacttcttggcataaaagcttcaatttcaatgcccctatctggaagccctaggcgccgcgcaaatgtaaaacattcgcttcgcttggcttgttatccaaaatagagtatggacctccgacagattggcaacccgtgggtaatcgaaaatggctccatctgcccctttgtcgaaggaatcaggaaacggccctcacctcctggcggagtgtagatatgtgaaagaatctaggcgacacttgcagactggacaacatgtgaacaaataagaccaacgttatggcaacaagcctcgacgctactcaagtggtgggaggccaccgcatgttccaacgaagcgccaaagaaagccttgcagactctaatgctattagtcgcctaggatatttggaatgaaaggaaccgcagagtttttcagcaccaagagcttccggtggctagtctgatagccaaaattaaggaggatgccaaaacatgggtcttggcgggcgcgaaacaccttgataggtggcttaccttttaacatgttcgggccaaaggccttgagacggtaaagttttctatttgcgcttgcgcatgtacaattttattcctctattcaatgaaattggtggctcactggttcattaaaaaaaaaagaatctagcctgttcgggaagaagaggattttattcgtgagagagagagagagagagagagagagagggagagagaaggaggaggaggattttcaggcttcgcattgcccaacctctgcttctgttggcccaagaagaatcccaggcgcccatgggctggcagtttaccacggacctacctagcctaccttagctatctaagcgggccgacctagtagctacgtgcctagtgtagattaaagttggcgggccagcaggaagccacgctgcaatggcatcttcccctgtccttcgcgtacgtgaaaacaaacccaggtaagcttagaatcttcttgcccgttggactgggacacccaccaatcccaccatgccccgatattcctccggtctcggttcatgtgatgtcctctcttgtgtgatcacggagcaagcattcttaaacggcaaaagaaaatcaccaacttgctcacgcagtcacgctgcaccgcgcgaagcgacgcccgataggccaagatcgcgagataaaataacaaccaatgatcataaggaaacaagcccgcgatgtgtcgtgtgcagcaatcttggtcatttgcgggatcgagtgcttcacggctaaccaaatattcggccgatgatttaacacattatcagcgtagatgtacgtacgatttgttaattaatctacgagccttgctagggcaggtgttctgccagccaatccagatcgccctcgtatgcacgctcacatgatggcagggcagggttcacatgagctctaacggtcgattaattaatcccggggctcgactataaatacctccctaatcccatgatcaaaaccatctcaagcagcctaatcatctccagctgatcaagagctcttaattagctagctagtgattagctgcgcttgtgatcgatcgatctcgggtacgtagcaatagatctaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcgg

SEQ ID NO: 97 - DNA, 2327 bp, synthetic construct
  source 1..2327: mol_type = other DNA; note = endosperm specific promoter
tgtttggactccagaaaatttacgggagttggtggagcaggtcattaagtactataaaaaatcatgtagctgaagctgcaagtatttagaagacatttagataagttattttatttatcatttagattaagaaaatttaaaactatttaaattgatattataaactacagctccacactggagctagatcctggagtcattacaaacacccccttaatgggaaaagagaagataatgtatatctaattattgtttctgtgtcacctatagctattagttcaaaacttcataatcactggtacaaataagctctagagaggcggttcggaacccatttttattgttgtttttcaaaaccactagtgttagggaccgccagtggaaactgaaacgccattggaaattgattttcactgatggtgagctaagaaaaccgccattggtaatcctttgcagaaaacataaactaggttttaaaaatagtaaacaaatatttttattaggagaggccccacatagtcgcaccatttttcgcgcattattcacgcgctacgcaaccaatggtaattgaacctcagagacttcactcttgtgtagcctcctttgccactccactaaacacttacttgtgtcttgattgcattttgttgcccacatattagaacaaacagagtgtaaattgattgtttgaggctgtaaacaaattcaaatgaaaaagtagtcaactactaaattgaataattgtttatgttctaccacttttattttggtacttttcccatcggaggcggtttgtaaaatttgcattttaagttttacaaatttcaatgaaattttgagagcccaaatgatttcaaataaaaaagttgtcaactacaatgttttataacttttaatttggtggttttttaaacaagctcatttgaaaaactaaaatgatcgattctacatgatttttaggtcgattttttaaggaatcgcctgtacaaatatttctactgacagtttttaagaaaccacctgtggaaatcatagatttgtactagcggtttttctcaagtaactgctagtagaaatatggtggttttcttaagaaaactgtttgtaggaatgcacgatttatataaatggatttgttaagaaaaccgctagtggaatgttctttcaactaacggttattgagtcgtgacagccaatttaatttccttgataactaaaagcggctgtaaaaattagaccatgatgtaggcacggagctgttttgtactgaatgcgcccactgttttgttggaaaagtgcatgtacttattattcattctgtttatttctagctggcattcagttcttacagccacagattatgcaaaacgcctatttctgccagcaaatttacaggaaaagtcatggacttttccgggttattttcctataagtacagccattcctttcacttacaggccccaacattagcacaaagaacacaatagaccactgatttaacacctgcaggaccaggtgggcccaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcgg

SEQ ID NO: 98 - DNA, 1713 bp, synthetic construct
  source 1..1713: mol_type = other DNA; note = endosperm specific promoter
atatagatttcttaacgacaatctcgcattgtttattgcctcacatggtgacacacagctcttacatttgtccttatactcacggtgcagtgcactactactgtaaccccatggtctggaaaacagcaacgcaacctttctgccttggaacagcaagctatacagcgctatataacaaacttgcacaataatatgctacatttctgaaagcaaactccgcttatcttcgcgttaaggaatggtattacacttaaaatgacaaaatatcacactttagaaccccacgcctttacctaaaaaaaagttgattgatacaagaacaaatgtttacagcttgttacacctcccatcaagcaaagaaatggaatcttatatagatttcttaacgacaatctcgcattgtttattgcctcacatggtgacacacagctcttacatttgtccttatactcacggtgcagtgcactactactgtaaccccatggtctggaaaacagcaacgcaacctttctgccttggaacagcaagctatacagcgctatataacaaacttgcacaataatatgctacatttctgaaagcaaactccgcttatcttcgcgttaaggaatggtattacacttaaaatgacaaaatatcacactttagaaccccacgcctttacctaaaaaaaagttgattgatacaagaacaaatgtttacagcttgttacacctcccatcaagcaaagaaatggaatcttttattttatgtacacgtgtacgggtagctattgtttataaattgcagaagacacctcaacatgatggataattgtatacgcaaaactactttcctcgaattccccattctataaattccagttaaacttaattcgtaagatcaatcataattctcgagttgcaaaccaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcgg

SEQ ID NO: 99 - DNA, 2516 bp, synthetic construct
  source 1..2516: mol_type = other DNA; note = seed specific promoter
acacttttattatcgcgtcaaatcagtacctcaatcgatattgtagcctagtgttcttattaaatgggaagaattcgaggacacactaattccttgctaacacacacttatgctccatttggatgtcgatattggagggcatggaactgaattggtttcaattacaaatcagccatgatattgtaatgagatgtaatttcaattctattctttggatgtcactgaattggagtttggaattgtgtggtccaattccaccttatatagaagagggatgctctgtattgggagagtgagtttctagttatagtctagcttcgggaaattgagtctctcgttccaaatctcaattccatgtgcaaccaaacaatagaattctggaaagctgattccaattcctaattccgtgctccaatatctacatccaaacgggtgttacataaatatagaaatgacatatcaaccatgcaaaaccacattggcgatgttgaacaaaggcgaacacccacatactatgtaccgcacacggcatctctttctcaaaggtcgaaccacgtgtgttccatgcatgcgtggaacatgcaaggttgtcacgtatagggaatgatgacacacgagagcgcctacaaggcaacaaacaccttacgtaccacgtagagtgcattttgctaccacctgccaccggatgacatgtatgcatgcatgcgttgtgtacgcatacactgctgtctgctggtgcccaaagaccatctagaacagcatcttttaattctccatttccctcacgccattgctagtgccttgcacatgctcgcactccctaacacatcttcctccctttatttttcgttgccaattgctagttgttcaaatgccacgttttccttacacagctgtagggcaccgtaccacgtagaatgcattcctcgccaccaacagacaacacggccgggcatatgtacgtcttacgccggaccatcaccagtatatatgatgctagggatcagtgggcgccctttttgcctcgtcctcccggggcggcattcctatgtcctaactgaagcaacccacgcgccgccatttctgttgcgaatgagtccatggacatatgtgccaacagaacccctcggaaggcaccatctatctatctatctctcaagcaatattatatttggcacctacgctcaagtacatagacagtgtgcacggcattgtgcagctggaaagcccgcccgacacgagggctgccaaatcgacagctccgcgcccttggaaatcctagtcacttgttcacaattgaccaatctacccttgaagcacacggtggatggtactgccacatttggcttataggggcatagaggacaatgaatgcaactggagcgggaaggagagctttaatttgtaagtactcggtgaacacggcacctgatgatgatgatgatggacagcgaggaattgttataaaaggcgcccgtccctcccatggctcaagaacaagggaatcgaagccattccctcttcaagaggggatcatcagattgggcttattattccttattactccaggtaattcttagtttgttgcccttccaaacctttacatctcatataagaatgattattacatgcaagattatgttgacatgcgtcgtcatggtattttttttaggcaaggatcggagttgctctgaattgactgaaccagatctaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcgg

SEQ ID NO: 100 - DNA, 2282 bp, synthetic construct
  source 1..2282: mol_type = other DNA; note = seed preferred promoter
gaggtgcataaggctggcaagacgacagttaggaacgcatgcgagcgaggttgacggacgcgataaggttagcgcatgcgtcgaacgcgggctggcgagggtggaaggcatgataaggctattgggtagcgcaaaatgtgtagacagcgggcgagtgaggatggcagtggtggcatgcatggacgcggttggagcatacgcgacaagaatggagcacgacgtagatttcgggaggccgtggttggagcgcccgcgggcgagatggcggccatggttagagcgcccgtggttacgggtgggttcatggcgagcggaggggttgcaagatttccagggcgctcgggtcggttgcaagctccacggtggaggcgtgacggagacgacgtggggagggaggtcgtggggaaattcggacgagcagaggcgtggcaggtgtggcatggggagggaggtcgcggggagggcgcagggaggtggcatggggagggaggctggggacgaagatgatgtgggcccagagggacgcgggacaaagaattgcgtatgataacgggttgattcgtagaattttaggcggtatttataaaaatgacgcaggacagccattggtactgatactttaatatagtagagaagagatataaattaggacgggtacaacaagaccacacgtactaacatttttttttgtcacaggctgctctaatacatatctctatgataagcgagctagggatgctagcgtgtccatttgattcctatataaatctccaattatagctgtagcaattaatttaataaacacccaacaatagatcaaatctcatagcaaatcataatcatgaatgctccaaaatcagctagctggctctcccttatcttcgtttttccttcttctcctgcaacgaaaagaaaaaaaaagaaaagaaaagaaaacggccgcttgtggtactaactcccaactacgcacctaccgcgcgcataactcttggccgcctgccctcatcacctccgcgtcgccgtcgactcatccttatcctccccatcacgctcaccccgcgcccgcaccgcgccatccgtactttcccggccgccccaccgctggccgccccgacgtgtcgcgccgccaccggaaggtcccgggccgtcgggcgggcagagcgcctgcagcggtggacccacgccacgctgacgcgggcgcgcgtccgtccaagaaacctgacgtaagcagtgacagaattggcgccgcctctcggcgtccacgtgtcgtggtcaacctgtcagagtggggctccgtgtgtgcgctaccgcaggggcccggcgcacgggccacacgtgtcgcggtcgaccgcggctataaatgcccggctccgcactcggaacaagtttcaagctctcctcccctcttcctaccattagcagtagccacagccagaacaccagcagacagcagcatcagcagggaggaacagcggcccaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcgg

SEQ ID NO: 101 - DNA, 1546 bp, synthetic construct
  source 1..1546: mol_type = other DNA; note = meristem and cob enhanced promoter
cctagcgtcccctgcccaaaagtgggctcaacaagcctaaatacatataatttataccacgtgcaacacatttattcatccatatcacatgtcatgcaaggcataagcatcatgttaacttagttatactgacatacatttatgagttgagatgtccaggatgtgagcgcatgagcccattgtccattcaggaccaagacaggctactaagcactttctacataacttgtatgtgctaactatagcatgcttatatggctctctccaaagttcaaagctagctcaaatcttttgatttaataaaacttaaatttgtttgatttcagataaactgataatttttataatatttagagtgagttgaaaacagaaactggccgcaaatccacctcaagccttttgatttgacctaaaaaaaagaagcccccacaaacaccactccacactagtgcactgtctctctccaaaggcagctgcattggcctccagccttttccctactgtgccgcgcgccctcccttctctctaataatagcatagggagagaaggcatactccgaggcatccttctcctttccctctccttccccaaacccttttcctctttccctcgccccaagaacttcatctcatctccaggcgccccttttgcgcttgcgcaggaggagctcacggggacagtgggcggagagctcgatcgctgcaccactacttcactggaggtccgcccactcccgggcccaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcgg

(19) United States
(12) Patent Application Publication
(10) Pub. No.: US 2024/0093221 A1
(43) Pub. Date: Mar. 21, 2024

(54) TRANSGENIC PLANTS WITH ENHANCED TRAITS

(71) Applicant: Monsanto Technology LLC, St. Louis, MO (US)

(72) Inventors: Edwards M. Allen, O'Fallon, MO (US); Bettina Darveaux, Hillsborough, NC (US); Stephen M. Duff, St. Louis, MO (US); Mary Fernandes, St. Louis, MO (US); Barry S. Goldman, St. Louis, MO (US); Cara L. Griffith, Catawissa, MO (US); Balasulojini Karunanandaa, Creve Coeur, MO (US); Saritha V. Kuriakose, Kottayam (IN); Paul J. Loida, Kirkwood, MO (US); Linda L. Lutfiyya, St. Louis, MO (US); Robert J. Meister, St. Peters, MO (US); Monnanda S. Rajani, Chesterfield, MO (US); Dhanalakshmi Ramachandra, Bangalore (IN); Elena A. Rice, Olivette, MO (US); Daniel Ruzicka, St. Louis, MO (US); Anagha M. Sant, St. Louis, MO (US); Jon J. Schmuke, St. Louis, MO (US); Rebecca L. Thompson, St. Charles, MO (US); Srikanth Babu Venkatachalayya, Bangalore (IN); Tyamagondlu V. Venkatesh, St. Louis, MO (US); Huai Wang, Chesterfield, MO (US); Xiao Yang, St. Charles, MO (US); Qin Zeng, Chesterfield, MO (US); Jianmin Zhao, St. Louis, MO (US)

(21) Appl. No.: 18/526,363

(22) Filed: Dec. 1, 2023

Related U.S. Application Data
(62) Division of application No. 17/694,370, filed on Mar. 14, 2022, now Pat. No. 11,873,502.
Application No. 17/694,370 is a division of application No. 16/085,527, filed on Sep. 14, 2018, now abandoned.
(60) Provisional application No. 62/310,136, filed on Mar. 18, 2016.

(51) Int. Cl. C12N 15/82; A01H 5/10; C12N 15/00

(52) U.S. Cl. CPC C12N 15/8273; A01H 5/10; C12N 15/00; C12N 15/8213; C12N 15/8218; C12N 15/825

(57) ABSTRACT

This disclosure provides recombinant DNA constructs and transgenic plants having enhanced traits such as increased yield, increased nitrogen use efficiency, and enhanced drought tolerance or water use efficiency. Transgenic plants may include field crops as well as plant propagules and progeny of such transgenic plants. Methods of making and using such transgenic plants are also provided. This disclosure also provides methods of producing seed from such transgenic plants, growing such seed, and selecting progeny plants with enhanced traits. Also disclosed are transgenic plants with altered phenotypes which are useful for screening and selecting transgenic events for the desired enhanced trait.

### INCORPORATION OF SEQUENCE LISTING

The sequence listing file named “61673US0000_ST25.txt”, which is 323 kilobytes (measured in MS-WINDOWS) and was created on Mar. 18, 2016, is filed herewith and incorporated herein by reference in its entirety.

### FIELD OF THE INVENTION

Disclosed herein are recombinant DNA constructs, plants having altered phenotypes, enhanced traits, increased yield, increased nitrogen use efficiency and increased water use efficiency; propagules, progenies and field crops of such plants; and methods of making and using such plants. Also disclosed are methods of producing seed from such plants, growing such seed and/or selecting progeny plants with altered phenotypes, enhanced traits, increased yield, increased nitrogen use efficiency and increased water use efficiency.

### SUMMARY

In one aspect, the present disclosure provides recombinant DNA constructs each comprising: a polynucleotide sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 1-29; or a polynucleotide sequence that encodes a polypeptide having an amino acid sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 30-92. The recombinant DNA construct may comprise a promoter, such as a heterologous promoter, functional in a plant cell and operably linked to the polynucleotide sequence. Vectors, plasmids, plants, propagules and plant cells are further provided comprising such a recombinant DNA construct.

Plants comprising a recombinant DNA construct may be a field crop plant, such as corn, soybean, cotton, canola, rice, barley, oat, wheat, turf grass, alfalfa, sugar beet, sunflower, quinoa and sugarcane. A plant comprising a recombinant DNA construct may have an altered phenotype or an enhanced trait as compared to a control plant. The enhanced trait may be, for example, decreased days from planting to maturity, increased stalk size, increased number of leaves, increased plant height growth rate in vegetative stage, increased ear size, increased ear dry weight per plant, increased number of kernels per ear, increased weight per kernel, increased number of kernels per plant, decreased ear void, extended grain fill period, reduced plant height, increased number of root branches, increased total root length, increased yield, increased nitrogen use efficiency, and increased water use efficiency as compared to a control plant. The altered phenotype may be, for example, plant height, biomass, canopy area, anthocyanin content, chlorophyll content, water applied, water content, and water use efficiency.

According to another aspect, the present disclosure provides methods for altering a phenotype, enhancing a trait, increasing yield, increasing nitrogen use efficiency, or increasing water use efficiency in a plant comprising producing a transgenic plant comprising a recombinant DNA construct, wherein the recombinant DNA construct comprises: a polynucleotide sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 1-29; or a polynucleotide sequence that encodes a polypeptide having an amino acid sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 30-92. The step of producing a transgenic plant may further comprise transforming a plant cell or tissue with the recombinant DNA construct, and regenerating or developing the transgenic plant from the plant cell or tissue comprising the recombinant DNA construct. The transgenic plant may then be crossed to (a) itself; (b) a second plant from the same plant line; (c) a wild type plant; or (d) a second plant from a different plant line, to produce one or more progeny plants; and a plant may be selected from the progeny plants having increased yield, increased nitrogen use efficiency, or increased water use efficiency as compared to a control plant. Plants produced by this method are further provided. According to some embodiments, the transgenic plant may be produced by site-directed integration of the recombinant DNA construct into the genome of a plant cell or tissue using a donor template comprising the recombinant DNA construct, and then regenerating or developing the transgenic plant from the transgenic plant cell or tissue.

According to another aspect, the present disclosure provides recombinant DNA molecules for use as a donor template in site-directed integration, wherein a recombinant DNA molecule comprises an insertion sequence comprising: a polynucleotide sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 1-29; or a polynucleotide sequence that encodes a polypeptide having an amino acid sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 30-92. The insertion sequence of the recombinant DNA molecule may comprise a promoter, such as a heterologous promoter, functional in a plant cell and operably linked to the polynucleotide sequence. The recombinant DNA molecule may further comprise at least one homology arm flanking the insertion sequence. Plants, propagules and plant cells are further provided comprising the insertion sequence. According to some embodiments, the recombinant DNA molecule may further comprise an expression cassette encoding a site-specific nuclease and/or one or more guide RNAs.

According to another aspect, the present disclosure provides recombinant DNA molecules for use as a donor template in site-directed integration, wherein a recombinant DNA molecule comprises an insertion sequence for modulation of expression of an endogenous gene, wherein the endogenous gene comprises: a polynucleotide sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 1-29, or a portion thereof; or a polynucleotide sequence that encodes a polypeptide having an amino acid sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 30-92. The insertion sequence may comprise a promoter, an enhancer, an intron, and/or a terminator region, which may correspond to a promoter, an enhancer, an intron, or a terminator region of an endogenous gene. Plants, propagules and plant cells are further provided comprising the insertion sequence. According to some embodiments, the recombinant DNA molecule may further comprise an expression cassette encoding a site-specific nuclease and/or one or more guide RNAs.

According to another aspect, the present disclosure provides methods for altering a phenotype, enhancing a trait, increasing yield, increasing nitrogen use efficiency, or increasing water use efficiency in a plant comprising: (a) modifying the genome of a plant cell by: (i) identifying an endogenous gene of the plant corresponding to a gene selected from the list of genes in Tables 1 and 12 herein, and their homologs, and (ii) modifying a sequence of the endogenous gene in the plant cell via site-directed integration to modify the expression level of the endogenous gene; and (b) regenerating or developing a plant from the plant cell.

### DETAILED DESCRIPTION

In the attached sequence listing:
SEQ ID NOs 1 to 29 are nucleotide sequences of the coding strand of the DNA used in the recombinant DNA constructs imparting an enhanced trait in plants, each representing a coding sequence for a protein.
SEQ ID NOs 30 to 58 are amino acid sequences of the cognate proteins of the DNA molecules with nucleotide sequences of SEQ ID NOs 1 to 29 respectively in the same order.
SEQ ID NOs 59 to 92 are amino acid sequences of proteins homologous to the proteins with amino acid sequences of SEQ ID NOs 30 to 58.
SEQ ID NOs 93 to 101 are nucleotide sequences of DNA molecules used in the recombinant DNA constructs imparting an enhanced trait or altered phenotype in plants, each representing a promoter with a specific expression pattern.

Unless otherwise stated, nucleic acid sequences in the text of this specification are given, when read from left to right, in the 5′ to 3′ direction. One of skill in the art would be aware that a given DNA sequence is understood to define a corresponding RNA sequence which is identical to the DNA sequence except for replacement of the thymine (T) nucleotide of the DNA with uracil (U) nucleotide. Thus, providing a specific DNA sequence is understood to define the exact RNA equivalent. A given first polynucleotide sequence, whether DNA or RNA, further defines the sequence of its exact complement (which can be DNA or RNA), i.e., a second polynucleotide that hybridizes perfectly to the first polynucleotide by forming Watson-Crick base-pairs. By “essentially identical” or “essentially complementary” to a target gene or a fragment of a target gene is meant that a polynucleotide strand (or at least one strand of a double-stranded polynucleotide) is designed to hybridize (generally under physiological conditions such as those found in a living plant or animal cell) to a target gene or to a fragment of a target gene or to the transcript of the target gene or the fragment of a target gene; one of skill in the art would understand that such hybridization does not necessarily require 100% sequence identity or complementarity. A first nucleic acid sequence is “operably” connected or “linked” with a second nucleic acid sequence when the first nucleic acid sequence is placed in a functional relationship with the second nucleic acid sequence. For example, a promoter sequence is “operably linked” to a DNA sequence if the promoter provides for transcription or expression of the DNA sequence. Generally, operably linked DNA sequences are contiguous.

As used herein, the term “expression” refers to the production of a polynucleotide or a protein by a plant, plant cell or plant tissue which can give rise to an altered phenotype or enhanced trait. Expression can also refer to the process by which information from a gene is used in the synthesis of functional gene products, which may include but are not limited to other polynucleotides or proteins which may serve, e.g., an enzymatic, structural or regulatory function. Gene products having a regulatory function include but are not limited to elements that affect the occurrence or level of transcription or translation of a target protein. In some cases, the expression product is a non-coding functional RNA.

“Modulation” of expression refers to the process of effecting either overexpression or suppression of a polynucleotide or a protein.

The term “suppression” as used herein refers to a lower expression level of a target polynucleotide or target protein in a plant, plant cell or plant tissue, as compared to the expression in a wild-type or control plant, cell or tissue, at any developmental or temporal stage for the gene. The term “target protein” as used in the context of suppression refers to a protein which is suppressed; similarly, “target mRNA” refers to a polynucleotide which can be suppressed or, once expressed, degraded so as to result in suppression of the target protein it encodes. The term “target gene” as used in the context of suppression refers to either “target protein” or “target mRNA”. In alternate non-limiting embodiments, suppression of the target protein or target polynucleotide can give rise to an enhanced trait or altered phenotype directly or indirectly. In one exemplary embodiment, the target protein is one which can indirectly increase or decrease the expression of one or more other proteins, the increased or decreased expression, respectively, of which is associated with an enhanced trait or an altered phenotype. In another exemplary embodiment, the target protein can bind to one or more other proteins associated with an altered phenotype or enhanced trait to enhance or inhibit their function and thereby affect the altered phenotype or enhanced trait indirectly.

Suppression can be applied using numerous approaches. Non-limiting examples include: suppressing an endogenous gene(s) or a subset of genes in a pathway, suppressing one or more mutation that has resulted in decreased activity of a protein, suppressing the production of an inhibitory agent, to elevate, reduce or eliminate the level of substrate that an enzyme requires for activity, producing a new protein, activating a normally silent gene; or accumulating a product that does not normally increase under natural conditions.

Conversely, the term “overexpression” as used herein refers to a greater expression level of a polynucleotide or a protein in a plant, plant cell or plant tissue, compared to expression in a wild-type plant, cell or tissue, at any developmental or temporal stage for the gene. Overexpression can take place in plant cells normally lacking expression of polypeptides functionally equivalent or identical to the present polypeptides. Overexpression can also occur in plant cells where endogenous expression of the present polypeptides or functionally equivalent molecules normally occurs, but such normal expression is at a lower level. Overexpression thus results in a greater than normal production, or “overproduction” of the polypeptide in the plant, cell or tissue.

The term “target protein” as used herein in the context of overexpression refers to a protein which is overexpressed; “target mRNA” refers to an mRNA which encodes and is translated to produce the target protein, which can also be overexpressed. The term “target gene” as used in the context of overexpression refers to either “target protein” or “target mRNA”. In alternative embodiments, the target protein can effect an enhanced trait or altered phenotype directly or indirectly. In the latter case it may do so, for example, by affecting the expression, function or substrate available to one or more other proteins. In an exemplary embodiment, the target protein can bind to one or more other proteins associated with an altered phenotype or enhanced trait to enhance or inhibit their function.

Overexpression can be achieved using numerous approaches. In one embodiment, overexpression can be achieved by placing the DNA sequence encoding one or more polynucleotides or polypeptides under the control of a promoter, examples of which include but are not limited to endogenous promoters, heterologous promoters, inducible promoters, development specific promoters, and tissue specific promoters. In one exemplary embodiment, the promoter is a constitutive promoter, for example, the cauliflower mosaic virus 35S promoter and other constitutive promoters known in the art. Thus, depending on the promoter used, overexpression can occur throughout a plant, in specific tissues of the plant, in specific stages of development of the plant, or in the presence or absence of different inducing or inducible agents, such as hormones or environmental signals.

Gene Suppression Elements: The gene suppression element can be transcribable DNA of any suitable length, and generally includes at least about 19 to about 27 nucleotides (for example 19, 20, 21, 22, 23, or 24 nucleotides) for every target gene that the recombinant DNA construct is intended to suppress. In many embodiments, the gene suppression element includes more than 23 nucleotides (for example, more than about 30, about 50, about 100, about 200, about 300, about 500, about 1000, about 1500, about 2000, about 3000, about 4000, or about 5000 nucleotides) for every target gene that the recombinant DNA construct is intended to suppress.

Suitable gene suppression elements useful in the recombinant DNA constructs of the invention include at least one element (and, in some embodiments, multiple elements) selected from the group consisting of: (a) DNA that includes at least one anti-sense DNA segment that is anti-sense to at least one segment of the at least one first target gene; (b) DNA that includes multiple copies of at least one anti-sense DNA segment that is anti-sense to at least one segment of the at least one first target gene; (c) DNA that includes at least one sense DNA segment that is at least one segment of the at least one first target gene; (d) DNA that includes multiple copies of at least one sense DNA segment that is at least one segment of the at least one first target gene; (e) DNA that transcribes to RNA for suppressing the at least one first target gene by forming double-stranded RNA and includes at least one anti-sense DNA segment that is anti-sense to at least one segment of the at least one target gene and at least one sense DNA segment that is at least one segment of the at least one first target gene; (f) DNA that transcribes to RNA for suppressing the at least one first target gene by forming a single double-stranded RNA and includes multiple serial anti-sense DNA segments that are anti-sense to at least one segment of the at least one first target gene and multiple serial sense DNA segments that are at least one segment of the at least one first target gene; (g) DNA that transcribes to RNA for suppressing the at least one first target gene by forming multiple double strands of RNA and includes multiple anti-sense DNA segments that are anti-sense to at least one segment of the at least one first target gene and multiple sense DNA segments that are at least one segment of the at least one first target gene, and wherein the multiple anti-sense DNA segments and the multiple sense DNA segments are arranged in a series of inverted repeats; (h) DNA that includes nucleotides derived from a miRNA, preferably a plant miRNA; (i) DNA that includes nucleotides of a siRNA; (j) DNA that transcribes to an RNA aptamer capable of binding to a ligand; and (k) DNA that transcribes to an RNA aptamer capable of binding to a ligand, and DNA that transcribes to regulatory RNA capable of regulating expression of the first target gene, wherein the regulation is dependent on the conformation of the regulatory RNA, and the conformation of the regulatory RNA is allosterically affected by the binding state of the RNA aptamer.

Any of these gene suppression elements, whether transcribing to a single double-stranded RNA or to multiple double-stranded RNAs, can be designed to suppress more than one target gene, including, for example, more than one allele of a target gene, multiple target genes (or multiple segments of at least one target gene) from a single species, or target genes from different species.

Anti-Sense DNA Segments: In one embodiment, the at least one anti-sense DNA segment that is anti-sense to at least one segment of the at least one first target gene includes DNA sequence that is anti-sense or complementary to at least a segment of the at least one first target gene, and can include multiple anti-sense DNA segments, that is, multiple copies of at least one anti-sense DNA segment that is anti-sense to at least one segment of the at least one first target gene. Multiple anti-sense DNA segments can include DNA sequence that is anti-sense or complementary to multiple segments of the at least one first target gene, or to multiple copies of a segment of the at least one first target gene, or to segments of multiple first target genes, or to any combination of these. Multiple anti-sense DNA segments can be fused into a chimera, e.g., including DNA sequences that are anti-sense to multiple segments of one or more first target genes and fused together.

The anti-sense DNA sequence that is anti-sense or complementary to (that is, can form Watson-Crick base-pairs with) at least a segment of the at least one first target gene has at least about 80%, or at least about 85%, or at least about 90%, or at least about 95% complementarity to at least a segment of the at least one first target gene. In one embodiment, the DNA sequence that is anti-sense or complementary to at least a segment of the at least one first target gene has between about 95% to about 100% complementarity to at least a segment of the at least one first target gene. Where the at least one anti-sense DNA segment includes multiple anti-sense DNA segments, the degree of complementarity can be, but need not be, identical for all of the multiple anti-sense DNA segments.

Sense DNA Segments: In another embodiment, the at least one sense DNA segment that is at least one segment of the at least one first target gene includes DNA sequence that corresponds to (that is, has a sequence that is identical or substantially identical to) at least a segment of the at least one first target gene, and can include multiple sense DNA segments, that is, multiple copies of at least one sense DNA segment that corresponds to (that is, has the nucleotide sequence of) at least one segment of the at least one first target gene. Multiple sense DNA segments can include DNA sequence that is or that corresponds to multiple segments of the at least one first target gene, or to multiple copies of a segment of the at least one first target gene, or to segments of multiple first target genes, or to any combination of these. Multiple sense DNA segments can be fused into a chimera, that is, can include DNA sequences corresponding to multiple segments of one or more first target genes and fused together.

The sense DNA sequence that corresponds to at least a segment of the target gene has at least about 80%, or at least about 85%, or at least about 90%, or at least about 95% sequence identity to at least a segment of the target gene. In one embodiment, the DNA sequence that corresponds to at least a segment of the target gene has between about 95% to about 100% sequence identity to at least a segment of the target gene. Where the at least one sense DNA segment includes multiple sense DNA segments, the degree of sequence identity can be, but need not be, identical for all of the multiple sense DNA segments.

Multiple Copies: Where the gene suppression element includes multiple copies of anti-sense or multiple copies of sense DNA sequence, these multiple copies can be arranged serially in tandem repeats. In some embodiments, these multiple copies can be arranged serially end-to-end, that is, in directly connected tandem repeats. In some embodiments, these multiple copies can be arranged serially in interrupted tandem repeats, where one or more spacer DNA segment can be located adjacent to one or more of the multiple copies. Tandem repeats, whether directly connected or interrupted or a combination of both, can include multiple copies of a single anti-sense or multiple copies of a single sense DNA sequence in a serial arrangement or can include multiple copies of more than one anti-sense DNA sequence or of more than one sense DNA sequence in a serial arrangement.

Double-stranded RNA: In those embodiments wherein the gene suppression element includes either at least one anti-sense DNA segment that is anti-sense to at least one segment of the at least one target gene or at least one sense DNA segment that is at least one segment of the at least one target gene, RNA transcribed from either the at least one anti-sense or at least one sense DNA may become double-stranded by the action of an RNA-dependent RNA polymerase. See, for example, U.S. Pat. No. 5,283,184, which is incorporated by reference herein.

In yet other embodiments, the gene suppression element can include DNA that transcribes to RNA for suppressing the at least one first target gene by forming double-stranded RNA and includes at least one anti-sense DNA segment that is anti-sense to at least one segment of the at least one target gene (as described above under the heading “Anti-sense DNA Segments”) and at least one sense DNA segment that is at least one segment of the at least one first target gene (as described above under the heading “Sense DNA Segments”). Such a gene suppression element can further include spacer DNA segments. Each at least one anti-sense DNA segment is complementary to at least part of a sense DNA segment in order to permit formation of double-stranded RNA by intramolecular hybridization of the at least one anti-sense DNA segment and the at least one sense DNA segment. Such complementarity between an anti-sense DNA segment and a sense DNA segment can be, but need not be, 100% complementary; in some embodiments, this complementarity can be preferably at least about 80%, or at least about 85%, or at least about 90%, or at least about 95% complementary.

The double-stranded RNA can be in the form of a single dsRNA “stem” (region of base-pairing between sense and anti-sense strands), or can have multiple dsRNA “stems.” In one embodiment, the gene suppression element can include DNA that transcribes to RNA for suppressing the at least one first target gene by forming essentially a single double-stranded RNA and includes multiple serial anti-sense DNA segments that are anti-sense to at least one segment of the at least one first target gene and multiple serial sense DNA segments that are at least one segment of the at least one first target gene; the multiple serial anti-sense and multiple serial sense segments can form a single double-stranded RNA “stem” or multiple “stems” in a serial arrangement (with or without non-base paired spacer DNA separating the multiple “stems”). In another embodiment, the gene suppression element includes DNA that transcribes to RNA for suppressing the at least one first target gene by forming multiple dsRNA “stems” of RNA and includes multiple anti-sense DNA segments that are anti-sense to at least one segment of the at least one first target gene and multiple sense DNA segments that are at least one segment of the at least one first target gene, and wherein the multiple anti-sense DNA segments and the multiple sense DNA segments are arranged in a series of dsRNA “stems” (such as, but not limited to “inverted repeats”). Such multiple dsRNA “stems” can further be arranged in series or clusters to form tandem inverted repeats, or structures resembling “hammerhead” or “cloverleaf” shapes. Any of these gene suppression elements can further include spacer DNA segments found within a dsRNA “stem” (for example, as a spacer between multiple anti-sense or sense DNA segments or as a spacer between a base-pairing anti-sense DNA segment and a sense DNA segment) or outside of a double-stranded RNA “stem” (for example, as a loop region separating a pair of inverted repeats). In cases where base-pairing anti-sense and sense DNA segments are of unequal length, the longer segment can act as a spacer.

miRNAs: In a further embodiment, the gene suppression element can include DNA that includes nucleotides derived from a miRNA (microRNA), that is, a DNA sequence that corresponds to a miRNA native to a virus or a eukaryote of interest (including plants and animals, especially invertebrates), or a DNA sequence derived from such a native miRNA but modified to include nucleotide sequences that do not correspond to the native miRNA. While miRNAs have not to date been reported in fungi, fungal miRNAs, should they exist, are also suitable for use in the invention. An embodiment includes a gene suppression element containing DNA that includes nucleotides derived from a viral or plant miRNA.

In a non-limiting example, the nucleotides derived from a miRNA can include DNA that includes nucleotides corresponding to the loop region of a native miRNA and nucleotides that are selected from a target gene sequence. In another non-limiting example, the nucleotides derived from a miRNA can include DNA derived from a miRNA precursor sequence, such as a native pri-miRNA or pre-miRNA sequence, or nucleotides corresponding to the regions of a native miRNA and nucleotides that are selected from a target gene sequence number such that the overall structure (e.g., the placement of mismatches in the stem structure of the pre-miRNA) is preserved to permit the pre-miRNA to be processed into a mature miRNA. In yet another embodiment, the gene suppression element can include DNA that includes nucleotides derived from a miRNA and capable of inducing or guiding in-phase cleavage of an endogenous transcript into trans-acting siRNAs, as described by Allen et al. (2005) Cell, 121:207-221. Thus, the DNA that includes nucleotides derived from a miRNA can include sequence naturally occurring in a miRNA or a miRNA precursor molecule, synthetic sequence, or both.

siRNAs: In yet another embodiment, the gene suppression element can include DNA that includes nucleotides of a small interfering RNA (siRNA). The siRNA can be one or more native siRNAs (such as siRNAs isolated from a non-transgenic eukaryote or from a transgenic eukaryote), or can be one or more DNA sequences predicted to have siRNA activity (such as by use of predictive tools known in the art, see, for example, Reynolds et al. (2004) Nature Biotechnol., 22:326-330). Multiple native or predicted siRNA sequences can be joined in a chimeric siRNA sequence for gene suppression. Such a DNA that includes nucleotides of a siRNA includes at least 19 nucleotides, and in some embodiments includes at least 20, at least 21, at least 22, at least 23, or at least 24 nucleotides. In other embodiments, the DNA that includes nucleotides of a siRNA can contain substantially more than 21 nucleotides, for example, more than about 50, about 100, about 300, about 500, about 1000, about 3000, or about 5000 nucleotides or greater.

Engineered miRNAs and trans-acting siRNAs (ta-siRNAs) are useful for gene suppression with increased specificity. The invention provides recombinant DNA constructs, each including a transcribable engineered miRNA precursor designed to suppress a target sequence, wherein the transcribable engineered miRNA precursor is derived from the fold-back structure of a MIR gene, preferably a plant MIR sequence. These miRNA precursors are also useful for directing in-phase production of siRNAs (e.g., heterologous sequence designed to be processed in a trans-acting siRNA suppression mechanism in planta). The invention further provides a method to suppress expression of a target sequence in a plant cell, including transcribing in a plant cell a recombinant DNA including a transcribable engineered miRNA precursor designed to suppress a target sequence, wherein the transcribable engineered miRNA precursor is derived from the fold-back structure of a MIR gene, preferably a plant MIR sequence, whereby expression of the target sequence is suppressed relative to its expression in the absence of transcription of the recombinant DNA construct.

The mature miRNAs produced, or predicted to be produced, from these miRNA precursors may be engineered for use in suppression of a target gene, e.g., in transcriptional suppression by the miRNA, or to direct in-phase production of siRNAs in a trans-acting siRNA suppression mechanism (see Allen et al. (2005) Cell, 121:207-221, Vaucheret (2005) Science STKE, 2005:pe43, and Yoshikawa et al. (2005) Genes Dev., 19:2164-2175). Plant miRNAs generally have near-perfect complementarity to their target sequences (see, for example, Llave et al. (2002) Science, 297:2053-2056, Rhoades et al. (2002) Cell, 110:513-520, Jones-Rhoades and Bartel (2004) Mol. Cell, 14:787-799). Thus, the mature miRNAs can be engineered to serve as sequences useful for gene suppression of a target sequence, by replacing nucleotides of the mature miRNA sequence with nucleotides of the sequence that is targeted for suppression; see, for example, methods disclosed by Parizotto et al. (2004) Genes Dev., 18:2237-2242 and especially U.S. Patent Application Publications US2004/0053411A1, US2004/0268441A1, US2005/0144669, and US2005/0037988, all of which are incorporated by reference herein. When engineering a novel miRNA to target a specific sequence, one strategy is to select within the target sequence a region with sequence that is as similar as possible to the native miRNA sequence. Alternatively, the native miRNA sequence can be replaced with a region of the target sequence, preferably a region that meets structural and thermodynamic criteria believed to be important for miRNA function (see, for example, U.S. Patent Application Publication US2005/0037988). Sequences are preferably engineered such that the number and placement of mismatches in the stem structure of the fold-back region or pre-miRNA is preserved. Thus, an engineered miRNA or engineered miRNA precursor can be derived from any of the mature miRNA sequences, or their corresponding miRNA precursors (including the fold-back portions of the corresponding MIR genes) disclosed herein. The engineered miRNA precursor can be cloned and expressed (transiently or stably) in a plant cell or tissue or intact plant.

The construction and description of recombinant DNA constructs to modulate small non-coding RNA activities are disclosed in U.S. Patent Application Publication US 2009/0070898 A1, US2011/0296555 A1, US2011/0035839 A1, all of which are incorporated herein by reference in their entirety. In particular, with respect to US2011/0035839 A1, see e.g., sections under the headings “Gene Suppression Elements” in paragraphs 122 to 135, and “Engineered Heterologous miRNA for Controlling Gene Expression in paragraphs 188 to 190.

As used herein a “plant” includes a whole plant, a transgenic plant, meristematic tissue, a shoot organ/structure (for example, leaf, stem and tuber), a root, a flower, a floral organ/structure (for example, a bract, a sepal, a petal, a stamen, a carpel, an anther and an ovule), a seed (including an embryo, endosperm, and a seed coat) and a fruit (the mature ovary), plant tissue (for example, vascular tissue, ground tissue, and the like) and a cell (for example, guard cell, egg cell, pollen, mesophyll cell, and the like), and progeny of same. The classes of plants that can be used in the disclosed methods are generally as broad as the classes of higher and lower plants amenable to transformation and breeding techniques, including angiosperms (monocotyledonous and dicotyledonous plants), gymnosperms, ferns, horsetails, psilophytes, lycophytes, bryophytes, and multicellular algae.

As used herein a “transgenic plant cell” means a plant cell that is transformed with stably-integrated, recombinant DNA, for example, by Agrobacterium-mediated transformation, by bombardment using microparticles coated with recombinant DNA, or by other means, such as site-directed integration. A plant cell of this disclosure can be an originally-transformed plant cell that exists as a microorganism or as a progeny plant cell that is regenerated into differentiated tissue, for example, into a transgenic plant with stably-integrated, recombinant DNA, or seed or pollen derived from a progeny transgenic plant.

As used herein a “control plant” means a plant that does not contain the recombinant DNA of the present disclosure that imparts an enhanced trait or altered phenotype. A control plant is used to identify and select a transgenic plant that has an enhanced trait or altered phenotype. A suitable control plant can be a non-transgenic plant of the parental line used to generate a transgenic plant, for example, a wild type plant devoid of a recombinant DNA. A suitable control plant can also be a transgenic plant that contains recombinant DNA that imparts other traits, for example, a transgenic plant having enhanced herbicide tolerance. A suitable control plant can in some cases be a progeny of a hemizygous transgenic plant line that does not contain the recombinant DNA, known as a negative segregant, or a negative isogenic line.

As used herein a “propagule” includes all products of meiosis and mitosis, including but not limited to, plant, seed and part of a plant able to propagate a new plant. Propagules include whole plants, cells, pollen, ovules, flowers, embryos, leaves, roots, stems, shoots, meristems, grains or seeds, or any plant part that is capable of growing into an entire plant. Propagule also includes graft where one portion of a plant is grafted to another portion of a different plant (even one of a different species) to create a living organism. Propagule also includes all plants and seeds produced by cloning or by bringing together meiotic products, or allowing meiotic products to come together to form an embryo or a fertilized egg (naturally or with human intervention).

As used herein a “progeny” includes any plant, seed, plant cell, and/or regenerable plant part comprising a recombinant DNA of the present disclosure derived from an ancestor plant. A progeny can be homozygous or heterozygous for the transgene. Progeny can be grown from seeds produced by a transgenic plant comprising a recombinant DNA of the present disclosure, and/or from seeds produced by a plant fertilized with pollen or ovule from a transgenic plant comprising a recombinant DNA of the present disclosure.

As used herein a “trait” is a physiological, morphological, biochemical, or physical characteristic of a plant or particular plant material or cell. In some instances, this characteristic is visible to the human eye and can be measured mechanically, such as seed or plant size, weight, shape, form, length, height, growth rate and development stage, or can be measured by biochemical techniques, such as detecting the protein, starch, certain metabolites, or oil content of seed or leaves, or by observation of a metabolic or physiological process, for example, by measuring tolerance to water deprivation or particular salt or sugar concentrations, or by the measurement of the expression level of a gene or genes, for example, by employing Northern analysis, RT-PCR, microarray gene expression assays, or reporter gene expression systems, or by agricultural observations such as hyperosmotic stress tolerance or yield. Any technique can be used to measure the amount of, comparative level of, or difference in any selected chemical compound or macromolecule in the transgenic plants, however.

As used herein an “enhanced trait” means a characteristic of a transgenic plant as a result of stable integration and expression of a recombinant DNA in the transgenic plant. Such traits include, but are not limited to, an enhanced agronomic trait characterized by enhanced plant morphology, physiology, growth and development, yield, nutritional enhancement, disease or pest resistance, or environmental or chemical tolerance. In some specific aspects of this disclosure an enhanced trait is selected from the group consisting of decreased days from planting to maturity, increased stalk size, increased number of leaves, increased plant height growth rate in vegetative stage, increased ear size, increased ear dry weight per plant, increased number of kernels per ear, increased weight per kernel, increased number of kernels per plant, decreased ear void, extended grain fill period, reduced plant height, increased number of root branches, increased total root length, drought tolerance, increased water use efficiency, cold tolerance, increased nitrogen use efficiency and increased yield as shown in Tables 7-10, and altered phenotypes as shown in Tables 3-5. In another aspect of the disclosure the trait is increased yield under non-stress conditions or increased yield under environmental stress conditions. Stress conditions can include both biotic and abiotic stress, for example, drought, shade, fungal disease, viral disease, bacterial disease, insect infestation, nematode infestation, cold temperature exposure, heat exposure, osmotic stress, reduced nitrogen nutrient availability, reduced phosphorus nutrient availability and high plant density. “Yield” can be affected by many properties including without limitation, plant height, plant biomass, pod number, pod position on the plant, number of internodes, incidence of pod shatter, grain size, ear size, ear tip filling, kernel abortion, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Yield can also be affected by efficiency of germination (including germination in stressed conditions), growth rate (including growth rate in stressed conditions), flowering time and duration, ear number, ear size, ear weight, seed number per ear or pod, seed size, composition of seed (starch, oil, protein) and characteristics of seed fill.

Also used herein, the term “trait modification” encompasses altering the naturally occurring trait by producing a detectable difference in a characteristic in a plant comprising a recombinant DNA of the present disclosure relative to a plant not comprising the recombinant DNA, such as a wild-type plant, or a negative segregant. In some cases, the trait modification can be evaluated quantitatively. For example, the trait modification can entail an increase or decrease, in an observed trait characteristics or phenotype as compared to a control plant. It is known that there can be natural variations in a modified trait. Therefore, the trait modification observed entails a change of the normal distribution and magnitude of the trait characteristics or phenotype in the plants as compared to a control plant.

The present disclosure relates to a plant with improved economically important characteristics, more specifically increased yield. More specifically the present disclosure relates to a plant comprising a polynucleotide of this disclosure, wherein the plant has increased yield as compared to a control plant. Many plants of this disclosure exhibited increased yield as compared to a control plant. In an embodiment, a plant of the present disclosure exhibited an improved trait that is related to yield, including but not limited to increased nitrogen use efficiency, increased nitrogen stress tolerance, increased water use efficiency and increased drought tolerance, as defined and discussed infra.

Yield can be defined as the measurable produce of economic value from a crop. Yield can be defined in the scope of quantity and/or quality. Yield can be directly dependent on several factors, for example, the number and size of organs, plant architecture (such as the number of branches, plant biomass, etc.), flowering time and duration, grain fill period. Root architecture and development, photosynthetic efficiency, nutrient uptake, stress tolerance, early vigor, delayed senescence and functional stay green phenotypes can be important factors in determining yield. Optimizing the above mentioned factors can therefore contribute to increasing crop yield.

Reference herein to an increase in yield-related traits can also be taken to mean an increase in biomass (weight) of one or more parts of a plant, which can include above ground and/or below ground (harvestable) plant parts. In particular, such harvestable parts are seeds, and performance of the methods of the disclosure results in plants with increased yield and in particular increased seed yield relative to the seed yield of suitable control plants. The term “yield” of a plant can relate to vegetative biomass (root and/or shoot biomass), to reproductive organs, and/or to propagules (such as seeds) of that plant.

Increased yield of a plant of the present disclosure can be measured in a number of ways, including test weight, seed number per plant, seed weight, seed number per unit area (for example, seeds, or weight of seeds, per acre), bushels per acre, tons per acre, or kilo per hectare. For example, corn yield can be measured as production of shelled corn kernels per unit of production area, for example in bushels per acre or metric tons per hectare. This is often also reported on a moisture adjusted basis, for example at 15.5 percent moisture. Increased yield can result from improved utilization of key biochemical compounds, such as nitrogen, phosphorous and carbohydrate, or from improved responses to environmental stresses, such as cold, heat, drought, salt, shade, high plant density, and attack by pests or pathogens. This disclosure can also be used to provide plants with improved growth and development, and ultimately increased yield, as the result of modified expression of plant growth regulators or modification of cell cycle or photosynthesis pathways. Also of interest is the generation of plants that demonstrate increased yield with respect to a seed component that may or may not correspond to an increase in overall plant yield.

In an embodiment, “alfalfa yield” can also be measured in forage yield, the amount of above ground biomass at harvest. Factors leading contributing to increased biomass include increased vegetative growth, branches, nodes and internodes, leaf area, and leaf area index.

In another embodiment, “canola yield” can also be measured in pod number, number of pods per plant, number of pods per node, number of internodes, incidence of pod shatter, seeds per silique, seed weight per silique, improved seed, oil, or protein composition.

Additionally, “corn or maize yield” can also be measured as production of shelled corn kernels per unit of production area, ears per acre, number of kernel rows per ear and number of kernels per row, kernel number or weight per ear, weight per kernel, ear number, ear weight, fresh or dry ear biomass (weight)

In yet another embodiment, “cotton yield” can be measured as bolls per plant, size of bolls, fiber quality, seed cotton yield in g/plant, seed cotton yield in lb/acre, lint yield in lb/acre, and number of bales.

Specific embodiment for “rice yield” can also include panicles per hill, grain per hill, and filled grains per panicle.

Still further embodiment for “soybean yield” can also include pods per plant, pods per acre, seeds per plant, seeds per pod, weight per seed, weight per pod, pods per node, number of nodes, and the number of internodes per plant.

In still further embodiment, “sugarcane yield” can be measured as cane yield (tons per acre; kg/hectare), total recoverable sugar (pounds per ton), and sugar yield (tons/acre).

In yet still further embodiment, “wheat yield” can include: cereal per unit area, grain number, grain weight, grain size, grains per head, seeds per head, seeds per plant, heads per acre, number of viable tillers per plant, composition of seed (for example, carbohydrates, starch, oil, and protein) and characteristics of seed fill.

The terms “yield”, “seed yield” are defined above for a number of core crops. The terms “increased”, “improved”, “enhanced” are interchangeable and are defined herein.

In another embodiment, the present disclosure provides a method for the production of plants having altered phenotype, enhanced trait, or increased yield; performance of the method gives plants altered phenotype, enhanced trait, or increased yield.

“Increased yield” can manifest as one or more of the following: (i) increased plant biomass (weight) of one or more parts of a plant, particularly aboveground (harvestable) parts, of a plant, increased root biomass (increased number of roots, increased root thickness, increased root length) or increased biomass of any other harvestable part; or (ii) increased early vigor, defined herein as an improved seedling aboveground area approximately three weeks post-germination. “Early vigor” refers to active healthy plant growth especially during early stages of plant growth, and can result from increased plant fitness due to, for example, the plants being better adapted to their environment (for example, optimizing the use of energy resources, uptake of nutrients and partitioning carbon allocation between shoot and root). Early vigor in corn, for example, is a combination of the ability of corn seeds to germinate and emerge after planting and the ability of the young corn plants to grow and develop after emergence. Plants having early vigor also show increased seedling survival and better establishment of the crop, which often results in highly uniform fields with the majority of the plants reaching the various stages of development at substantially the same time, which often results in increased yield. Therefore early vigor can be determined by measuring various factors, such as kernel weight, percentage germination, percentage emergence, seedling growth, seedling height, root length, root and shoot biomass, canopy size and color and others.

Further, increased yield can also manifest as (iii) increased total seed yield, which may result from one or more of an increase in seed biomass (seed weight) due to an increase in the seed weight on a per plant and/or on an individual seed basis an increased number of panicles per plant; an increased number of pods; an increased number of nodes; an increased number of flowers (“florets”) per panicle/plant; increased seed fill rate; an increased number of filled seeds; increased seed size (length, width, area, perimeter), which can also influence the composition of seeds; and/or increased seed volume, which can also influence the composition of seeds.

Increased yield can also (iv) result in modified architecture, or can occur because of modified plant architecture.

Increased yield can also manifest as (v) increased harvest index, which is expressed as a ratio of the yield of harvestable parts, such as seeds, over the total biomass

Increased yield can also manifest as (vi) increased kernel weight, which is extrapolated from the number of filled seeds counted and their total weight. An increased kernel weight can result from an increased seed size and/or seed weight, an increase in embryo size, increased endosperm size, aleurone and/or scutellum, or an increase with respect to other parts of the seed that result in increased kernel weight.

Increased yield can also manifest as (vii) increased ear biomass, which is the weight of the ear and can be represented on a per ear, per plant or per plot basis.

In one embodiment, increased yield can be increased seed yield, and is selected from one of the following: (i) increased seed weight; (ii) increased number of filled seeds; and (iii) increased harvest index.

The disclosure also extends to harvestable parts of a plant such as, but not limited to, seeds, leaves, fruits, flowers, bolls, stems, rhizomes, tubers and bulbs. The disclosure furthermore relates to products derived from a harvestable part of such a plant, such as dry pellets, powders, oil, fat and fatty acids, starch or proteins.

The present disclosure provides a method for increasing “yield” of a plant or “broad acre yield” of a plant or plant part defined as the harvestable plant parts per unit area, for example seeds, or weight of seeds, per acre, pounds per acre, bushels per acre, tones per acre, tons per acre, kilo per hectare.

This disclosure further provides a method of altering phenotype, enhancing trait, or increasing yield in a plant by producing a plant comprising a polynucleotide sequence of this disclosure where the plant can be crossed with itself, a second plant from the same plant line, a wild type plant, or a plant from a different line of plants to produce a seed. The seed of the resultant plant can be harvested from fertile plants and be used to grow progeny generations of plant(s) of this disclosure. In addition to direct transformation of a plant with a recombinant DNA construct, transgenic plants can be prepared by crossing a first plant having a stably integrated recombinant DNA construct with a second plant lacking the DNA. For example, recombinant DNA can be introduced into a first plant line that is amenable to transformation to produce a transgenic plant which can be crossed with a second plant line to introgress the recombinant DNA into the second plant line.

Selected transgenic plants transformed with a recombinant DNA construct and having the polynucleotide of this disclosure provides the altered phenotype, enhanced trait, or increased yield compared to a control plant. Use of genetic markers associated with the recombinant DNA can facilitate production of transgenic progeny that is homozygous for the desired recombinant DNA. Progeny plants carrying DNA for both parental traits can be back-crossed into a parent line multiple times, for example usually 6 to 8 generations, to produce a progeny plant with substantially the same genotype as the one reoccurring original transgenic parental line but having the recombinant DNA of the other transgenic parental line. The term “progeny” denotes the offspring of any generation of a parent plant prepared by the methods of this disclosure containing the recombinant polynucleotides as described herein.

As used herein “nitrogen use efficiency” refers to the processes which lead to an increase in the plant's yield, biomass, vigor, and growth rate per nitrogen unit applied. The processes can include the uptake, assimilation, accumulation, signaling, sensing, retranslocation (within the plant) and use of nitrogen by the plant.

As used herein “nitrogen limiting conditions” refers to growth conditions or environments that provide less than optimal amounts of nitrogen needed for adequate or successful plant metabolism, growth, reproductive success and/or viability.

As used herein the “increased nitrogen stress tolerance” refers to the ability of plants to grow, develop, or yield normally, or grow, develop, or yield faster or better when subjected to less than optimal amounts of available/applied nitrogen, or under nitrogen limiting conditions.

As used herein “increased nitrogen use efficiency” refers to the ability of plants to grow, develop, or yield faster or better than normal when subjected to the same amount of available/applied nitrogen as under normal or standard conditions; ability of plants to grow, develop, or yield normally, or grow, develop, or yield faster or better when subjected to less than optimal amounts of available/applied nitrogen, or under nitrogen limiting conditions.

Increased plant nitrogen use efficiency can be translated in the field into either harvesting similar quantities of yield, while supplying less nitrogen, or increased yield gained by supplying optimal/sufficient amounts of nitrogen. The increased nitrogen use efficiency can improve plant nitrogen stress tolerance, and can also improve crop quality and biochemical constituents of the seed such as protein yield and oil yield. The terms “increased nitrogen use efficiency”, “enhanced nitrogen use efficiency”, and “nitrogen stress tolerance” are used inter-changeably in the present disclosure to refer to plants with improved productivity under nitrogen limiting conditions.

As used herein “water use efficiency” refers to the amount of carbon dioxide assimilated by leaves per unit of water vapor transpired. It constitutes one of the most important traits controlling plant productivity in dry environments. “Drought tolerance” refers to the degree to which a plant is adapted to arid or drought conditions. The physiological responses of plants to a deficit of water include leaf wilting, a reduction in leaf area, leaf abscission, and the stimulation of root growth by directing nutrients to the underground parts of the plants. Plants are more susceptible to drought during flowering and seed development (the reproductive stages), as plant's resources are deviated to support root growth. In addition, abscisic acid (ABA), a plant stress hormone, induces the closure of leaf stomata (microscopic pores involved in gas exchange), thereby reducing water loss through transpiration, and decreasing the rate of photosynthesis. These responses improve the water-use efficiency of the plant on the short term. The terms “increased water use efficiency”, “enhanced water use efficiency”, and “increased drought tolerance” are used inter-changeably in the present disclosure to refer to plants with improved productivity under water-limiting conditions.

As used herein “increased water use efficiency” refers to the ability of plants to grow, develop, or yield faster or better than normal when subjected to the same amount of available/applied water as under normal or standard conditions; ability of plants to grow, develop, or yield normally, or grow, develop, or yield faster or better when subjected to reduced amounts of available/applied water (water input) or under conditions of water stress or water deficit stress.

As used herein “increased drought tolerance” refers to the ability of plants to grow, develop, or yield normally, or grow, develop, or yield faster or better than normal when subjected to reduced amounts of available/applied water and/or under conditions of acute or chronic drought; ability of plants to grow, develop, or yield normally when subjected to reduced amounts of available/applied water (water input) or under conditions of water deficit stress or under conditions of acute or chronic drought.

As used herein “drought stress” refers to a period of dryness (acute or chronic/prolonged) that results in water deficit and subjects plants to stress and/or damage to plant tissues and/or negatively affects grain/crop yield; a period of dryness (acute or chronic/prolonged) that results in water deficit and/or higher temperatures and subjects plants to stress and/or damage to plant tissues and/or negatively affects grain/crop yield.

As used herein “water deficit” refers to the conditions or environments that provide less than optimal amounts of water needed for adequate/successful growth and development of plants.

As used herein “water stress” refers to the conditions or environments that provide improper (either less/insufficient or more/excessive) amounts of water than that needed for adequate/successful growth and development of plants/crops thereby subjecting the plants to stress and/or damage to plant tissues and/or negatively affecting grain/crop yield.

As used herein “water deficit stress” refers to the conditions or environments that provide less/insufficient amounts of water than that needed for adequate/successful growth and development of plants/crops thereby subjecting the plants to stress and/or damage to plant tissues and/or negatively affecting grain yield.

As used herein a “polynucleotide” is a nucleic acid molecule comprising a plurality of polymerized nucleotides. A polynucleotide may be referred to as a nucleic acid, a oligonucleotide, or any fragment thereof. In many instances, a polynucleotide encodes a polypeptide (or protein) or a domain or a fragment thereof. Additionally, a polynucleotide can comprise a promoter, an intron, an enhancer region, a polyadenylation site, a translation initiation site, 5′ or 3′ untranslated regions, a reporter gene, a selectable marker, a scorable marker, or the like. A polynucleotide can be single-stranded or double-stranded DNA or RNA. A polynucleotide optionally comprises modified bases or a modified backbone. A polynucleotide can be, for example, genomic DNA or RNA, a transcript (such as an mRNA), a cDNA, a PCR product, a cloned DNA, a synthetic DNA or RNA, or the like. A polynucleotide can be combined with carbohydrate(s), lipid(s), protein(s), or other materials to perform a particular activity, such as transformation, or form a composition such as a peptide nucleic acid (PNA). A polynucleotide can comprise a sequence in either sense or antisense orientations. “Oligonucleotide” is substantially equivalent to the terms amplimer, primer, oligomer, element, target, and probe and is preferably single-stranded.

As used herein a “recombinant polynucleotide” or “recombinant DNA” is a polynucleotide that is not in its native state, for example, a polynucleotide comprises a series of nucleotides (represented as a nucleotide sequence) not found in nature, or a polynucleotide is in a context other than that in which it is naturally found; for example, separated from polynucleotides with which it typically is in proximity in nature, or adjacent (or contiguous with) polynucleotides with which it typically is not in proximity The “recombinant polynucleotide” or “recombinant DNA” refers to polynucleotide or DNA which has been genetically engineered and constructed outside of a cell including DNA containing naturally occurring DNA or cDNA or synthetic DNA. For example, the polynucleotide at issue can be cloned into a vector, or otherwise recombined with one or more additional nucleic acids.

As used herein a “polypeptide” comprises a plurality of consecutive polymerized amino acid residues for example, at least about 15 consecutive polymerized amino acid residues. In many instances, a polypeptide comprises a series of polymerized amino acid residues that is a transcriptional regulator or a domain or portion or fragment thereof. Additionally, the polypeptide can comprise: (i) a localization domain; (ii) an activation domain; (iii) a repression domain; (iv) an oligomerization domain; (v) a protein-protein interaction domain; (vi) a DNA-binding domain; or the like. The polypeptide optionally comprises modified amino acid residues, naturally occurring amino acid residues not encoded by a codon, non-naturally occurring amino acid residues.

As used herein “protein” refers to a series of amino acids, oligopeptide, peptide, polypeptide or portions thereof whether naturally occurring or synthetic.

As used herein a “recombinant polypeptide” is a polypeptide produced by translation of a recombinant polynucleotide.

A “synthetic polypeptide” is a polypeptide created by consecutive polymerization of isolated amino acid residues using methods known in the art.

An “isolated polypeptide”, whether a naturally occurring or a recombinant polypeptide, is more enriched in (or out of) a cell than the polypeptide in its natural state in a wild-type cell, for example, more than about 5% enriched, more than about 10% enriched, or more than about 20%, or more than about 50%, or more, enriched, for example, alternatively denoted: 105%, 110%, 120%, 150% or more, enriched relative to wild type standardized at 100%. Such enrichment is not the result of a natural response of a wild-type plant. Alternatively, or additionally, the isolated polypeptide is separated from other cellular components, with which it is typically associated, for example, by any of the various protein purification methods.

As used herein, a “functional fragment” refers to a portion of a polypeptide provided herein which retains full or partial molecular, physiological or biochemical function of the full length polypeptide. A functional fragment often contains the domain(s), such as Pfam domains (see below), identified in the polypeptide provided in the sequence listing.

A “recombinant DNA construct” as used in the present disclosure comprises at least one expression cassette having a promoter operable in plant cells and a polynucleotide of the present disclosure. DNA constructs can be used as a means of delivering recombinant DNA constructs to a plant cell in order to effect stable integration of the recombinant molecule into the plant cell genome. In one embodiment, the polynucleotide can encode a protein or variant of a protein or fragment of a protein that is functionally defined to maintain activity in transgenic host cells including plant cells, plant parts, explants and whole plants. In another embodiment, the polynucleotide can encode a non-coding RNA that interferes with the functioning of endogenous classes of small RNAs that regulate expression, including but not limited to taRNAs, siRNAs and miRNAs. Recombinant DNA constructs are assembled using methods known to persons of ordinary skill in the art and typically comprise a promoter operably linked to DNA, the expression of which provides the enhanced agronomic trait.

Other construct components can include additional regulatory elements, such as 5′ leaders and introns for enhancing transcription, 3′ untranslated regions (such as polyadenylation signals and sites), and DNA for transit or targeting or signal peptides.

Percent identity describes the extent to which polynucleotides or protein segments are invariant in an alignment of sequences, for example nucleotide sequences or amino acid sequences. An alignment of sequences is created by manually aligning two sequences, for example, a stated sequence, as provided herein, as a reference, and another sequence, to produce the highest number of matching elements, for example, individual nucleotides or amino acids, while allowing for the introduction of gaps into either sequence. An “identity fraction” for a sequence aligned with a reference sequence is the number of matching elements, divided by the full length of the reference sequence, not including gaps introduced by the alignment process into the reference sequence. “Percent identity” (“% identity”) as used herein is the identity fraction times 100.

As used herein, a “homolog” or “homologues” means a protein in a group of proteins that perform the same biological function, for example, proteins that belong to the same Pfam protein family and that provide a common enhanced trait in transgenic plants of this disclosure. Homologs are expressed by homologous genes. With reference to homologous genes, homologs include orthologs, for example, genes expressed in different species that evolved from common ancestral genes by speciation and encode proteins retain the same function, but do not include paralogs, i.e., genes that are related by duplication but have evolved to encode proteins with different functions. Homologous genes include naturally occurring alleles and artificially-created variants.

Degeneracy of the genetic code provides the possibility to substitute at least one base of the protein encoding sequence of a gene with a different base without causing the amino acid sequence of the polypeptide produced from the gene to be changed. When optimally aligned, homolog proteins, or their corresponding nucleotide sequences, have typically at least about 60% identity, in some instances at least about 70%, at least about 75%, at least about 80%, at least about 85%, at least about 90%, at least about 92%, at least about 93%, at least about 94%, at least about 95%, at least about 96%, at least about 97%, at least about 98%, at least about 99%, or even at least about 99.5% identity over the full length of a protein or its corresponding nucleotide sequence identified as being associated with imparting an enhanced trait or altered phenotype when expressed in plant cells. In one aspect of the disclosure homolog proteins have at least about 80%, at least about 85%, at least about 90%, at least about 92%, at least about 93%, at least about 94%, at least about 95%, at least about 96%, at least about 97%, at least about 98%, at least about 99%, or at least about 99.5% identity to a consensus amino acid sequence of proteins and homologs that can be built from sequences disclosed herein.

Homologs are inferred from sequence similarity, by comparison of protein sequences, for example, manually or by use of a computer-based tool using known sequence comparison algorithms such as BLAST and FASTA. A sequence search and local alignment program, for example, BLAST, can be used to search query protein sequences of a base organism against a database of protein sequences of various organisms, to find similar sequences, and the summary Expectation value (E-value) can be used to measure the level of sequence similarity. Because a protein hit with the lowest E-value for a particular organism may not necessarily be an ortholog or be the only ortholog, a reciprocal query is used to filter hit sequences with significant E-values for ortholog identification. The reciprocal query entails search of the significant hits against a database of protein sequences of the base organism. A hit can be identified as an ortholog, when the reciprocal query's best hit is the query protein itself or a paralog of the query protein. With the reciprocal query process orthologs are further differentiated from paralogs among all the homologs, which allows for the inference of functional equivalence of genes. A further aspect of the homologs encoded by DNA useful in the transgenic plants of the invention are those proteins that differ from a disclosed protein as the result of deletion or insertion of one or more amino acids in a native sequence.

Other functional homolog proteins differ in one or more amino acids from those of a trait-improving protein disclosed herein as the result of one or more of known conservative amino acid substitutions, for example, valine is a conservative substitute for alanine and threonine is a conservative substitute for serine. Conservative substitutions for an amino acid within the native sequence can be selected from other members of a class to which the naturally occurring amino acid belongs. Representative amino acids within these various classes include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, tyrosine, asparagine, and glutamine; and (4) neutral nonpolar (hydrophobic) amino acids such as alanine, leucine, isoleucine, valine, proline, phenylalanine, tryptophan, and methionine. Conserved substitutes for an amino acid within a native protein or polypeptide can be selected from other members of the group to which the naturally occurring amino acid belongs. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side 30 chains is cysteine and methionine. Naturally conservative amino acids substitution groups are: valine-leucine, valine-isoleucine, phenylalanine-tyrosine, lysine-arginine, alaninevaline, aspartic acid-glutamic acid, and asparagine-glutamine. A further aspect of the disclosure includes proteins that differ in one or more amino acids from those of a described protein sequence as the result of deletion or insertion of one or more amino acids in a native sequence.

In general, the term “variant” refers to molecules with some differences, generated synthetically or naturally, in their nucleotide or amino acid sequences as compared to a reference (native) polynucleotides or polypeptides, respectively. These differences include substitutions, insertions, deletions or any desired combinations of such changes in a native polynucleotide or amino acid sequence.

With regard to polynucleotide variants, differences between presently disclosed polynucleotides and polynucleotide variants are limited so that the nucleotide sequences of the former and the latter are similar overall and, in many regions, identical. Due to the degeneracy of the genetic code, differences between the former and the latter nucleotide sequences may be silent (for example, the amino acids encoded by the polynucleotide are the same, and the variant polynucleotide sequence encodes the same amino acid sequence as the presently disclosed polynucleotide). Variant nucleotide sequences can encode different amino acid sequences, in which case such nucleotide differences will result in amino acid substitutions, additions, deletions, insertions, truncations or fusions with respect to the similarly disclosed polynucleotide sequences. These variations can result in polynucleotide variants encoding polypeptides that share at least one functional characteristic. The degeneracy of the genetic code also dictates that many different variant polynucleotides can encode identical and/or substantially similar polypeptides.

As used herein “gene” or “gene sequence” refers to the partial or complete coding sequence of a gene, its complement, and its 5′ and/or 3′ untranslated regions (UTRs) and their complements. A gene is also a functional unit of inheritance, and in physical terms is a particular segment or sequence of nucleotides along a molecule of DNA (or RNA, in the case of RNA viruses) involved in producing a polypeptide chain. The latter can be subjected to subsequent processing such as chemical modification or folding to obtain a functional protein or polypeptide. By way of example, a transcriptional regulator gene encodes a transcriptional regulator polypeptide, which can be functional or require processing to function as an initiator of transcription.

As used herein, the term “promoter” refers generally to a DNA molecule that is involved in recognition and binding of RNA polymerase II and other proteins (trans-acting transcription factors) to initiate transcription. A promoter can be initially isolated from the 5′ untranslated region (5′ UTR) of a genomic copy of a gene. Alternately, promoters can be synthetically produced or manipulated or engineered DNA molecules. Promoters can also be chimeric, that is a promoter produced through the fusion of two or more heterologous DNA molecules. Plant promoters include promoter DNA obtained from plants, plant viruses, fungi and bacteria such as Agrobacterium and Bradyrhizobium bacteria. A promoter may also be heterologous. As used herein, a promoter or other regulatory sequence operably linked to a transcribable DNA sequence, such as a coding sequence, is considered “heterologous” if in nature the promoter or regulatory sequence is not operably linked to the transcribable DNA sequence and/or is not present in the plant host cell to be transformed with the promoter or regulatory sequence. Two or more promoter or regulatory sequences may also be heterologous with respect to each other.

Promoters which initiate transcription in all or most tissues of the plant are referred to as “constitutive” promoters. Promoters which initiate transcription during certain periods or stages of development are referred to as “developmental” promoters. Promoters whose expression is enhanced in certain tissues of the plant relative to other plant tissues are referred to as “tissue enhanced” or “tissue preferred” promoters. Promoters which express within a specific tissue of the plant, with little or no expression in other plant tissues are referred to as “tissue specific” promoters. A promoter that expresses in a certain cell type of the plant, for example a microspore mother cell, is referred to as a “cell type specific” promoter. An “inducible” promoter is a promoter in which transcription is initiated in response to an environmental stimulus such as cold, drought or light; or other stimuli such as wounding or chemical application. Many physiological and biochemical processes in plants exhibit endogenous rhythms with a period of about 24 hours. A “diurnal promoter” is a promoter which exhibits altered expression profiles under the control of a circadian oscillator. Diurnal regulation is subject to environmental inputs such as light and temperature and coordination by the circadian clock. Many examples of plant expressible promoters are known in the art.

Sufficient expression in plant seed tissues is desired to affect improvements in seed composition. Exemplary promoters for use for seed composition modification include promoters from seed genes such as napin as disclosed in U.S. Pat. No. 5,420,034, maize L3 oleosin as disclosed in U.S. Pat. No. 6,433,252, zein Z27 as disclosed by Russell et al. (1997) Transgenic Res. 6 (2):157-166, globulin 1 as disclosed by Belanger et al (1991) Genetics 129:863-872, glutelin 1 as disclosed by Russell (1997) supra, and peroxiredoxin antioxidant (Perl) as disclosed by Stacy et al. (1996) Plant Mol Biol. 31 (6):1205-1216.

As used herein, the term “leader” refers to a DNA molecule isolated from the untranslated 5′ region (5′ UTR) of a genomic copy of a gene and is defined generally as a nucleotide segment between the transcription start site (TSS) and the protein coding sequence start site. Alternately, leaders can be synthetically produced or manipulated DNA elements. A leader can be used as a 5′ regulatory element for modulating expression of an operably linked transcribable polynucleotide molecule. As used herein, the term “intron” refers to a DNA molecule that can be isolated or identified from the genomic copy of a gene and can be defined generally as a region spliced out during mRNA processing prior to translation. Alternately, an intron can be a synthetically produced or manipulated DNA element. An intron can contain enhancer elements that effect the transcription of operably linked genes. An intron can be used as a regulatory element for modulating expression of an operably linked transcribable polynucleotide molecule. A DNA construct can comprise an intron, and the intron may or may not be with respect to the transcribable polynucleotide molecule.

As used herein, the term “enhancer” or “enhancer element” refers to a cis-acting transcriptional regulatory element, a.k.a. cis-element, which confers an aspect of the overall expression pattern, but is usually insufficient alone to drive transcription, of an operably linked polynucleotide. Unlike promoters, enhancer elements do not usually include a transcription start site (TSS) or TATA box or equivalent sequence. A promoter can naturally comprise one or more enhancer elements that affect the transcription of an operably linked polynucleotide. An isolated enhancer element can also be fused to a promoter to produce a chimeric promoter cis-element, which confers an aspect of the overall modulation of gene expression. A promoter or promoter fragment can comprise one or more enhancer elements that effect the transcription of operably linked genes. Many promoter enhancer elements are believed to bind DNA-binding proteins and/or affect DNA topology, producing local conformations that selectively allow or restrict access of RNA polymerase to the DNA template or that facilitate selective opening of the double helix at the site of transcriptional initiation. An enhancer element can function to bind transcription factors that regulate transcription. Some enhancer elements bind more than one transcription factor, and transcription factors can interact with different affinities with more than one enhancer domain.

Expression cassettes of this disclosure can include a “transit peptide” or “targeting peptide” or “signal peptide” molecule located either 5′ or 3′ to or within the gene(s). These terms generally refer to peptide molecules that when linked to a protein of interest directs the protein to a particular tissue, cell, subcellular location, or cell organelle. Examples include, but are not limited to, chloroplast transit peptides (CTPs), chloroplast targeting peptides, mitochondrial targeting peptides, nuclear targeting signals, nuclear exporting signals, vacuolar targeting peptides, and vacuolar sorting peptides. For description of the use of chloroplast transit peptides see U.S. Pat. Nos. 5,188,642 and 5,728,925. For description of the transit peptide region of an Arabidopsis EPSPS gene in the present disclosure, see Klee, H. J. Et al (MGG (1987) 210:437-442. Expression cassettes of this disclosure can also include an intron or introns. Expression cassettes of this disclosure can contain a DNA near the 3′ end of the cassette that acts as a signal to terminate transcription from a heterologous nucleic acid and that directs polyadenylation of the resultant mRNA. These are commonly referred to as “3′-untranslated regions” or “3′-non-coding sequences” or “3′-UTRs”. The “3′ non-translated sequences” means DNA sequences located downstream of a structural nucleotide sequence and include sequences encoding polyadenylation and other regulatory signals capable of affecting mRNA processing or gene expression. The polyadenylation signal functions in plants to cause the addition of polyadenylate nucleotides to the 3′ end of the mRNA precursor. The polyadenylation signal can be derived from a natural gene, from a variety of plant genes, or from T-DNA. An example of a polyadenylation sequence is the nopaline synthase 3′ sequence (nos 3′; Fraley et al., Proc. Natl. Acad. Sci. USA 80: 4803-4807, 1983). The use of different 3′ non-translated sequences is exemplified by Ingelbrecht et al., Plant Cell 1:671-680, 1989.

Expression cassettes of this disclosure can also contain one or more genes that encode selectable markers and confer resistance to a selective agent such as an antibiotic or an herbicide. A number of selectable marker genes are known in the art and can be used in the present disclosure: selectable marker genes conferring tolerance to antibiotics like kanamycin and paromomycin (nptII), hygromycin B (aph IV), spectinomycin (aadA), U.S. Patent Publication 2009/0138985A1 and gentamycin (aac3 and aacC4) or tolerance to herbicides like glyphosate (for example, 5-enolpyruvylshikimate-3-phosphate synthase (EPSPS), U.S. Pat. Nos. 5,627,061; 5,633,435; 6,040,497; 5,094,945), sulfonyl herbicides (for example, acetohydroxyacid synthase or acetolactate synthase conferring tolerance to acetolactate synthase inhibitors such as sulfonylurea, imidazolinone, triazolopyrimidine, pyrimidyloxybenzoates and phthalide (U.S. Pat. Nos. 6,225,105; 5,767,366; 4,761,373; 5,633,437; 6,613,963; 5,013,659; 5,141,870; 5,378,824; 5,605,011)), bialaphos or phosphinothricin or derivatives (e. g., phosphinothricin acetyltransferase (bar) tolerance to phosphinothricin or glufosinate (U.S. Pat. Nos. 5,646,024; 5,561,236; 5,276,268; 5,637,489; 5,273,894); dicamba (dicamba monooxygenase, Patent Application Publications US2003/0115626A1), or sethoxydim (modified acetyl-coenzyme A carboxylase for conferring tolerance to cyclohexanedione), and aryloxyphenoxypropionate (haloxyfop, U.S. Pat. No. 6,414,222).

Transformation vectors of this disclosure can contain one or more “expression cassettes”, each comprising a native or non-native plant promoter operably linked to a polynucleotide sequence of interest, which is operably linked to a 3′ UTR sequence and termination signal, for expression in an appropriate host cell. It also typically comprises sequences required for proper translation of the polynucleotide or transgene. As used herein, the term “transgene” refers to a polynucleotide molecule artificially incorporated into a host cell's genome. Such a transgene can be heterologous to the host cell. The term “transgenic plant” refers to a plant comprising such a transgene. The coding region usually codes for a protein of interest but can also code for a functional RNA of interest, for example an antisense RNA, a nontranslated RNA, in the sense or antisense direction, a miRNA, a noncoding RNA, or a synthetic RNA used in either suppression or over expression of target gene sequences. The expression cassette comprising the nucleotide sequence of interest can be chimeric, meaning that at least one of its components is heterologous with respect to at least one of its other components. As used herein the term “chimeric” refers to a DNA molecule that is created from two or more genetically diverse sources, for example a first molecule from one gene or organism and a second molecule from another gene or organism.

Recombinant DNA constructs in this disclosure generally include a 3′ element that typically contains a polyadenylation signal and site. Known 3′ elements include those from Agrobacterium tumefaciens genes such as nos 3′, tml 3′, tmr 3′, tins 3′, ocs 3′, tr7 3′, for example disclosed in U.S. Pat. No. 6,090,627; 3′ elements from plant genes such as wheat (Triticum aesevitum) heat shock protein 17 (Hsp17 3′), a wheat ubiquitin gene, a wheat fructose-1,6-biphosphatase gene, a rice glutelin gene, a rice lactate dehydrogenase gene and a rice beta-tubulin gene, all of which are disclosed in U.S. Patent Application Publication 2002/0192813 A1; and the pea (Pisum sativum) ribulose biphosphate carboxylase gene (rbs 3′), and 3′ elements from the genes within the host plant.

As an alternative to traditional transformation methods, a DNA sequence, such as a transgene, expression cassette(s), etc., may be inserted or integrated into a specific site or locus within the genome of a plant or plant cell via site-directed integration. Recombinant DNA construct(s) and molecule(s) of this disclosure may thus include a donor template sequence comprising at least one transgene, expression cassette, or other DNA sequence for insertion into the genome of the plant or plant cell. Such donor template for site-directed integration may further include one or two homology arms flanking an insertion sequence (i.e., the sequence, transgene, cassette, etc., to be inserted into the plant genome). The recombinant DNA construct(s) of this disclosure may further comprise an expression cassette(s) encoding a site-specific nuclease and/or any associated protein(s) to carry out site-directed integration. These nuclease expressing cassette(s) may be present in the same molecule or vector as the donor template (in cis) or on a separate molecule or vector (in trans). Several methods for site-directed integration are known in the art involving different proteins (or complexes of proteins and/or guide RNA) that cut the genomic DNA to produce a double strand break (DSB) or nick at a desired genomic site or locus. Briefly as understood in the art, during the process of repairing the DSB or nick introduced by the nuclease enzyme, the donor template DNA may become integrated into the genome at the site of the DSB or nick. The presence of the homology arm(s) in the donor template may promote the adoption and targeting of the insertion sequence into the plant genome during the repair process through homologous recombination, although an insertion event may occur through non-homologous end joining (NHEJ). Examples of site-specific nucleases that may be used include zinc-finger nucleases, engineered or native meganucleases, TALE-endonucleases, and RNA-guided endonucleases (e.g., Cas9 or Cpf1). For methods using RNA-guided site-specific nucleases (e.g., Cas9 or Cpf1), the recombinant DNA construct(s) will also comprise a sequence encoding one or more guide RNAs to direct the nuclease to the desired site within the plant genome.

As used herein, the term “homology arm” refers to a polynucleotide sequence that has at least 80%, at least 85%, at least 90%, at least 91%, at least 92%, at least 93%, at least 94%, at least 95%, at least 96%, at least 97%, at least 98%, at least 99% or 100% sequence identity to a target sequence in a plant or plant cell that is being transformed. A homology arm can comprise at least 15, at least 20, at least 25, at least 30, at least 40, at least 50, at least 100, at least 250, at least 500, or at least 1000 nucleotides.

As used herein “operably linked” means the association of two or more DNA fragments in a recombinant DNA construct so that the function of one, for example, protein-encoding DNA, is controlled by the other, for example, a promoter.

Transgenic plants can comprise a stack of one or more polynucleotides disclosed herein resulting in the production of multiple polypeptide sequences. Transgenic plants comprising stacks of polynucleotides can be obtained by either or both of traditional breeding methods or through genetic engineering methods. These methods include, but are not limited to, crossing individual transgenic lines each comprising a polynucleotide of interest, transforming a transgenic plant comprising a first gene disclosed herein with a second gene, and co-transformation of genes into a single plant cell. Co-transformation of genes can be carried out using single transformation vectors comprising multiple genes or genes carried separately on multiple vectors.

Transgenic plants comprising or derived from plant cells of this disclosure transformed with recombinant DNA can be further enhanced with stacked traits, for example, a crop plant having an enhanced trait resulting from expression of DNA disclosed herein in combination with herbicide and/or pest resistance traits. For example, genes of the current disclosure can be stacked with other traits of agronomic interest, such as a trait providing herbicide resistance, or insect resistance, such as using a gene from Bacillus thuringensis to provide resistance against lepidopteran, coliopteran, homopteran, hemiopteran, and other insects, or improved quality traits such as improved nutritional value. Herbicides for which transgenic plant tolerance has been demonstrated and the method of the present disclosure can be applied include, but are not limited to, glyphosate, dicamba, glufosinate, sulfonylurea, bromoxynil, norflurazon, 2,4-D (2,4-dichlorophenoxy) acetic acid, aryloxyphenoxy propionates, p-hydroxyphenyl pyruvate dioxygenase inhibitors (HPPD), and protoporphyrinogen oxidase inhibitors (PPO) herbicides. Polynucleotide molecules encoding proteins involved in herbicide tolerance known in the art and include, but are not limited to, a polynucleotide molecule encoding 5-enolpyruvylshikimate-3-phosphate synthase (EPSPS) disclosed in U.S. Pat. Nos. 5,094,945; 5,627,061; 5,633,435 and 6,040,497 for imparting glyphosate tolerance; polynucleotide molecules encoding a glyphosate oxidoreductase (GOX) disclosed in U.S. Pat. No. 5,463,175 and a glyphosate-N-acetyl transferase (GAT) disclosed in U.S. Patent No. Application Publication 2003/0083480 A1 also for imparting glyphosate tolerance; dicamba monooxygenase disclosed in U.S. Patent Application Publication 2003/0135879 A1 for imparting dicamba tolerance; a polynucleotide molecule encoding bromoxynil nitrilase (Bxn) disclosed in U.S. Pat. No. 4,810,648 for imparting bromoxynil tolerance; a polynucleotide molecule encoding phytoene desaturase (crtI) described in Misawa et al, (1993) Plant J. 4:833-840 and in Misawa et al, (1994) Plant J. 6:481-489 for norflurazon tolerance; a polynucleotide molecule encoding acetohydroxyacid synthase (AHAS, aka ALS) described in Sathasiivan et al. (1990) Nucl. Acids Res. 18:2188-2193 for imparting tolerance to sulfonylurea herbicides; polynucleotide molecules known as bar genes disclosed in DeBlock, et al. (1987) EMBO J. 6:2513-2519 for imparting glufosinate and bialaphos tolerance; polynucleotide molecules disclosed in U.S. Patent Application Publication 2003/010609 A1 for imparting N-amino methyl phosphonic acid tolerance; polynucleotide molecules disclosed in U.S. Pat. No. 6,107,549 for imparting pyridine herbicide resistance; molecules and methods for imparting tolerance to multiple herbicides such as glyphosate, atrazine, ALS inhibitors, isoxoflutole and glufosinate herbicides are disclosed in U.S. Pat. No. 6,376,754 and U.S. Patent Application Publication 2002/0112260. Molecules and methods for imparting insect/nematode/virus resistance are disclosed in U.S. Pat. Nos. 5,250,515; 5,880,275; 6,506,599; 5,986,175 and U.S. Patent Application Publication 2003/0150017 A1.

### Plant Cell Transformation Methods

Numerous methods for transforming chromosomes and plastids in a plant cell with recombinant DNA are known in the art that may be used in methods of producing a transgenic plant cell and plant. Two effective methods for transformation are Agrobacterium-mediated transformation and microprojectile bombardment-mediated transformation. Microprojectile bombardment methods are illustrated, for example, in U.S. Pat. No. 5,015,580 (soybean); U.S. Pat. No. 5,550,318 (corn); U.S. Pat. No. 5,538,880 (corn); U.S. Pat. No. 5,914,451 (soybean); U.S. Pat. No. 6,160,208 (corn); U.S. Pat. No. 6,399,861 (corn); U.S. Pat. No. 6,153,812 (wheat) and U.S. Pat. No. 6,365,807 (rice). Agrobacterium-mediated transformation methods are described, for example, in U.S. Pat. No. 5,159,135 (cotton); U.S. Pat. No. 5,824,877 (soybean); U.S. Pat. No. 5,463,174 (canola); U.S. Pat. No. 5,591,616 (corn); U.S. Pat. No. 5,846,797 (cotton); U.S. Pat. No. 8,044,260 (cotton); U.S. Pat. No. 6,384,301 (soybean), U.S. Pat. No. 7,026,528 (wheat) and U.S. Pat. No. 6,329,571 (rice), U.S. Patent Application Publication No. 2004/0087030 A1 (cotton), and U.S. Patent Application Publication No. 2001/0042257 A1 (sugar beet), all of which are incorporated herein by reference in their entirety. Transformation of plant material is practiced in tissue culture on nutrient media, for example a mixture of nutrients that allow cells to grow in vitro. Recipient cell targets include, but are not limited to, meristem cells, shoot tips, hypocotyls, calli, immature or mature embryos, and gametic cells such as microspores, pollen, sperm and egg cells. Callus can be initiated from tissue sources including, but not limited to, immature or mature embryos, hypocotyls, seedling apical meristems, microspores and the like. Cells containing a transgenic nucleus are grown into transgenic plants.

As introduced above, another method for transforming chromosomes in a plant cell is via insertion of a DNA sequence using a recombinant DNA donor template at a pre-determined site of the genome by methods of site-directed integration. Site-directed integration may be accomplished by any method known in the art, for example, by use of zinc-finger nucleases, engineered or native meganucleases, TALE-endonucleases, or an RNA-guided endonuclease (for example Cas9 or Cpf1). The recombinant DNA construct may be inserted at the pre-determined site by homologous recombination (HR) or by non-homologous end joining (NHEJ). In addition to insertion of a recombinant DNA construct into a plant chromosome at a pre-determined site, genome editing can be achieved through oligonucleotide-directed mutagenesis (ODM) (Oh and May, 2001; U.S. Pat. No. 8,268,622) or by introduction of a double-strand break (DSB) or nick with a site specific nuclease, followed by NHEJ or repair. The repair of the DSB or nick may be used to introduce insertions or deletions at the site of the DSB or nick, and these mutations may result in the introduction of frame-shifts, amino acid substitutions, and/or an early termination codon of protein translation or alteration of a regulatory sequence of a gene. Genome editing may be achieved with or without a donor template molecule.

In addition to direct transformation of a plant material with a recombinant DNA construct, a transgenic plant can be prepared by crossing a first plant comprising a recombinant DNA with a second plant lacking the recombinant DNA. For example, recombinant DNA can be introduced into a first plant line that is amenable to transformation, which can be crossed with a second plant line to introgress the recombinant DNA into the second plant line. A transgenic plant with recombinant DNA providing an enhanced trait, for example, enhanced yield, can be crossed with a transgenic plant line having other recombinant DNA that confers another trait, for example herbicide resistance or pest resistance, to produce progeny plants having recombinant DNA that confers both traits. Typically, in such breeding for combining traits the transgenic plant donating the additional trait is a male line and the transgenic plant carrying the base traits is the female line. The progeny of this cross will segregate such that some of the plants will carry the DNA for both parental traits and some will carry DNA for one parental trait; such plants can be identified by markers associated with parental recombinant DNA, for example, marker identification by analysis for recombinant DNA or, in the case where a selectable marker is linked to the recombinant, by application of the selecting agent such as a herbicide for use with a herbicide tolerance marker, or by selection for the enhanced trait. Progeny plants carrying DNA for both parental traits can be crossed back into the female parent line multiple times, for example usually 6 to 8 generations, to produce a progeny plant with substantially the same genotype as the original transgenic parental line but for the recombinant DNA of the other transgenic parental line.

For transformation, DNA is typically introduced into only a small percentage of target plant cells in any one transformation experiment. Marker genes are used to provide an efficient system for identification of those cells that are stably transformed by receiving and integrating a recombinant DNA construct into their genomes. Preferred marker genes provide selective markers which confer resistance to a selective agent, such as an antibiotic or an herbicide. Any of the herbicides to which plants of this disclosure can be resistant is an agent for selective markers. Potentially transformed cells are exposed to the selective agent. In the population of surviving cells are those cells where, generally, the resistance-conferring gene is integrated and expressed at sufficient levels to permit cell survival. Cells can be tested further to confirm stable integration of the exogenous DNA. Commonly used selective marker genes include those conferring resistance to antibiotics such as kanamycin and paromomycin (nptII), hygromycin B (aph IV), spectinomycin (aadA) and gentamycin (aac3 and aacC4) or resistance to herbicides such as glufosinate (bar or pat), dicamba (DMO) and glyphosate (aroA or EPSPS). Examples of such selectable markers are illustrated in U.S. Pat. Nos. 5,550,318; 5,633,435; 5,780,708; 6,118,047 and 8,030,544. Markers which provide an ability to visually screen transformants can also be employed, for example, a gene expressing a colored or fluorescent protein such as a luciferase or green fluorescent protein (GFP) or a gene expressing a beta-glucuronidase or uidA gene (GUS) for which various chromogenic substrates are known.

Plant cells that survive exposure to a selective agent, or plant cells that have been scored positive in a screening assay, may be cultured in vitro to develop or regenerate plantlets. Developing plantlets regenerated from transformed plant cells can be transferred to plant growth mix, and hardened off, for example, in an environmentally controlled chamber at about 85% relative humidity, 600 ppm CO₂, and 25-250 microeinsteins m⁻² s⁻¹ of light, prior to transfer to a greenhouse or growth chamber for maturation. Plants may be regenerated from about 6 weeks to 10 months after a transformant is identified, depending on the initial tissue, and plant species. Plants can be pollinated using conventional plant breeding methods known to those of skill in the art to produce seeds, for example self-pollination is commonly used with transgenic corn. The regenerated transformed plant or its progeny seed or plants can be tested for expression of the recombinant DNA and selected for the presence of an altered phenotype or an enhanced agronomic trait.

### Transgenic Plants and Seeds

Transgenic plants derived from transgenic plant cells having a transgenic nucleus of this disclosure are grown to generate transgenic plants having an altered phenotype or an enhanced trait as compared to a control plant, and produce transgenic seed and haploid pollen of this disclosure. Such plants with enhanced traits are identified by selection of transformed plants or progeny seed for the enhanced trait. For efficiency a selection method is designed to evaluate multiple transgenic plants (events) comprising the recombinant DNA, for example multiple plants from 2 to 20 or more transgenic events. Transgenic plants grown from transgenic seeds provided herein demonstrate improved agronomic traits that contribute to increased yield or other traits that provide increased plant value, including, for example, improved seed quality. Of particular interest are plants having increased water use efficiency or drought tolerance, enhanced high temperature or cold tolerance, increased yield, and increased nitrogen use efficiency.

Table 1 provides a list of sequences of protein-encoding genes as recombinant DNA for production of transgenic plants with enhanced traits. The elements of Table 1 are described by reference to: “NUC SEQ ID NO.” which identifies a DNA sequence; “PEP SEQ ID NO.” which identifies an amino acid sequence; “Gene ID” which refers to an arbitrary identifier; and “Gene Name and Description” which is a common name and functional description of the gene.

TABLE 1
Sequences for Protein-Coding Genes

| NUC | PEP | | |
|---|---|---|---|
| SEQ ID | SEQ ID | | |
| NO. | NO. | Gene ID | Gene Name and Description |
| 1 | 30 | T5MON01 | Agrobacterium ornithine carbamoyl transferase (argF) |
| 2 | 31 | T5MON02 | Arabidopsis MADS-box protein (AGL20) |
| 3 | 32 | T5MON03 | Arabidopsis containing Pleckstrin homology domain, 6 regulator of |
| | | | chromosome condensation (RCC1) domains, and Zinc finger domain |
| 4 | 33 | T5MON04 | Arabidopsis Glycyl-tRNA synthetase |
| 5 | 34 | T5MON05 | Arabidopsis L-ascorbate oxidase |
| 6 | 35 | T5MON06 | Arabidopsis glutamate decarboxylase 4 (GAD4) |
| 7 | 36 | T5MON07 | Arabidopsis growth factor like protein |
| 8 | 37 | T5MON08 | Arabidopsis Aromatic and neutral amino acid transporter 1-like |
| | | | (ANT1-like) |
| 9 | 38 | T5MON09 | Arabidopsis putative AP2/EREBP transcription factor |
| | | | (with A33V mutation) |
| 10 | 39 | T5MON10 | Arabidopsis dark inducible 11 (DIN11) |
| 11 | 40 | T5MON11 | Arabidopsis strictosidine synthase family protein, mucin-like (AtSSL7) |
| 12 | 41 | T5MON12 | Arabidopsis purple acid phosphatase precursor (AtPAP) |
| 13 | 42 | T5MON13 | Arabidopsis serine/threonine protein kinase, root hair specific 3 (RHS3) |
| 14 | 43 | T5MON14 | Chlamydomonas reinhardtii PHE0023587_Ferredoxin-dependent |
| | | | glutamate synthase (GSF1) |
| 15 | 44 | T5MON15 | Chlorella sorokiniana NADP-specific glutamate dehydrogenase |
| | | | (NADP-GDH), N terminus residues 1 to 74 truncated |
| 16 | 45 | T5MON16 | E coli glutamate dehydrogenase, NADP-specific (gdhA) |
| 17 | 46 | T5MON17 | Homeobox fusion protein, Arabidopsis HB-17 N terminus residues 1 |
| | | | to 91 fused to soybean HB-17 C terminus residues 20-213 |
| 18 | 47 | T5MON18 | Soybean AP2-EREBP transcription factor (ERF3) |
| 19 | 48 | T5MON19 | rice Ghd7 |
| 20 | 49 | T5MON20 | Corn pyruvate orthophosphate dikinase 1 (PPDK1) |
| 21 | 50 | T5MON21 | Corn proliferating cell nuclear antigen (PCNA2) |
| 22 | 51 | T5MON23 | Corn Roothairless 1 (RTH1) |
| 23 | 52 | T5MON24 | Corn orphans transcription factor (Orphan46) |
| 24 | 53 | T5MON25 | Corn MADS transcription factor (MADS12) |
| 25 | 54 | T5MON26 | Corn remorin like DNA-binding protein 2 |
| 26 | 55 | T5MON27 | Corn CAAT-box DNA binding protein (NFBa) |
| 27 | 56 | T5MON28 | Corn homeobox-leucine zipper protien, HB transcription factor |
| 28 | 57 | T5MON29 | Corn putative glutamine synthetase (with C299A mutation) |
| 29 | 58 | T5MON30 | Corn putative nucleostemin |

### Selecting and Testing Transgenic Plants for Enhanced Traits

Within a population of transgenic plants each developed or regenerated from a plant cell with recombinant DNA many plants that survive to fertile transgenic plants that produce seeds and progeny plants will not exhibit an enhanced agronomic trait. Selection from the population is necessary to identify one or more transgenic plants with an enhanced trait. Further evaluation with vigorous testing is essential for understanding the contributing components to a trait, supporting trait advancement decisions and generating mode of action hypotheses. Transgenic plants having enhanced traits are selected and tested from populations of plants developed, regenerated or derived from plant cells transformed as described herein by evaluating the plants in a variety of assays to detect an enhanced trait, for example, increased water use efficiency or drought tolerance, enhanced high temperature or cold tolerance, increased yield or yield components, desirable architecture, optimum life cycle, increased nitrogen use efficiency, enhanced seed composition such as enhanced seed protein and enhanced seed oil.

These assays can take many forms including, but not limited to, direct screening for the trait in a greenhouse or field trial or by screening for a surrogate trait. Such analyses can be directed to detecting changes in the chemical composition, biomass, yield components, physiological property, root architecture, morphology, or life cycle of the plant. Changes in chemical compositions such as nutritional composition of grain can be detected by analysis of the seed composition and content of protein, free amino acids, oil, free fatty acids, starch or tocopherols. Changes in chemical compositions can also be detected by analysis of contents in leaves, such as chlorophyll or carotenoid contents. Changes in biomass characteristics can be evaluated on greenhouse or field grown plants and can include plant height, stem diameter, root and shoot dry weights, canopy size; and, for corn plants, ear length and diameter. Changes in yield components can be measured by total number of kernels per unit area and its individual weight. Changes in physiological properties can be identified by evaluating responses to stress conditions, for example assays using imposed stress conditions such as water deficit, nitrogen deficiency, cold growing conditions, pathogen or insect attack or light deficiency, or increased plant density. Changes in root architecture can be evaluated by root length and branch number. Changes in morphology can be measured by visual observation of tendency of a transformed plant to appear to be a normal plant as compared to changes toward bushy, taller, thicker, narrower leaves, striped leaves, knotted trait, chlorosis, albino, anthocyanin production, or altered tassels, ears or roots. Changes in morphology can also be measured with morphometric analysis based on shape parameters, using dimensional measurement such as ear diameter, ear length, kernel row number, internode length, plant height, or stem volume. Changes in life cycle can be measured by macro or microscopic morphological changes partitioned into developmental stages, such as days to pollen shed, days to silking, leaf extension rate. Other selection and testing properties include days to pollen shed, days to silking, leaf extension rate, chlorophyll content, leaf temperature, stand, seedling vigor, internode length, plant height, leaf number, leaf area, tillering, brace roots, stay green or delayed senescence, stalk lodging, root lodging, plant health, bareness/prolificacy, green snap, and pest resistance. In addition, phenotypic characteristics of harvested grain can be evaluated, including number of kernels per row on the ear, number of rows of kernels on the ear, kernel abortion, kernel weight, kernel size, kernel density and physical grain quality.

Assays for screening for a desired trait are readily designed by those practicing in the art. The following illustrates screening assays for corn traits using hybrid corn plants. The assays can be adapted for screening other plants such as canola, wheat, cotton and soybean either as hybrids or inbreds.

Transgenic corn plants having increased nitrogen use efficiency can be identified by screening transgenic plants in the field under the same and sufficient amount of nitrogen supply as compared to control plants, where such plants provide higher yield as compared to control plants. Transgenic corn plants having increased nitrogen use efficiency can also be identified by screening transgenic plants in the field under reduced amount of nitrogen supply as compared to control plants, where such plants provide the same or similar yield as compared to control plants.

Transgenic corn plants having increased yield are identified by screening using progenies of the transgenic plants over multiple locations for several years with plants grown under optimal production management practices and maximum weed and pest control or standard agronomic practices (SAP). Selection methods can be applied in multiple and diverse geographic locations, for example up to 16 or more locations, over one or more planting seasons, for example at least two planting seasons, to statistically distinguish yield improvement from natural environmental effects.

Transgenic corn plants having increased water use efficiency or drought tolerance are identified by screening plants in an assay where water is withheld for a period to induce stress followed by watering to revive the plants. For example, a selection process imposes 3 drought/re-water cycles on plants over a total period of 15 days after an initial stress free growth period of 11 days. Each cycle consists of 5 days, with no water being applied for the first four days and a water quenching on the 5th day of the cycle. The primary phenotypes analyzed by the selection method are the changes in plant growth rate as determined by height and biomass during a vegetative drought treatment.

Although the plant cells and methods of this disclosure can be applied to any plant cell, plant, seed or pollen, for example, any fruit, vegetable, grass, tree or ornamental plant, the various aspects of the disclosure are applied to corn, soybean, cotton, canola, rice, barley, oat, wheat, turf grass, alfalfa, sugar beet, sunflower, quinoa and sugar cane plants.

EXAMPLES

### Example 1. Corn Transformation

This example illustrates transformation methods to produce a transgenic corn plant cell, seed, and plant having altered phenotypes as shown in Tables 3-5, and enhanced traits, increased water use efficiency, increased nitrogen use efficiency, and increased yield and altered traits and phenology as shown in Tables 7, 8 and 10.

For Agrobacterium-mediated transformation of corn embryo cells, ears from corn plants were harvested and surface-sterilized by spraying or soaking the ears in ethanol, followed by air drying. Embryos were isolated from individual kernels of surface-sterilized ears. After excision, maize embryos were inoculated with Agrobacterium cells containing plasmid DNA with the gene of interest cassette and a plant selectable marker cassette, and then co-cultured with Agrobacterium for several days. Co-cultured embryos were transferred to various selection and regeneration media, and transformed R0 plants were recovered 6 to 8 weeks after initiation of selection, which were transplanted into potting soil. Regenerated R0 plants were selfed, and R1 and subsequent progeny generations were obtained.

The above process can be repeated to produce multiple events of transgenic corn plants from cells that were transformed with recombinant DNA having the genes identified in Table 1. Progeny transgenic plants and seeds of the transformed plants were screened for the presence and single copy of the inserted gene, and for various altered or enhanced traits and phenotypes, such as increased water use efficiency, increased yield, and increased nitrogen use efficiency as shown in Tables 3-5, 7, 8 and 10. From each group of multiple events of transgenic plants with a specific recombinant DNA from Table 1, the event(s) that showed increased yield, increased water use efficiency, increased nitrogen use efficiency, and altered phenotypes and traits were identified.

### Example 2. Soybean Transformation

This example illustrates plant transformation in producing a transgenic soybean plant cell, seed, and plant having an altered phenotype or an enhanced trait, such as increased water use efficiency, drought tolerance and increased yield as shown in Tables 9 and 10.

For Agrobacterium mediated transformation, soybean seeds were imbibed overnight and the meristem explants excised. Soybean explants were mixed with induced Agrobacterium cells containing plasmid DNA with the gene of interest cassette and a plant selectable marker cassette no later than 14 hours from the time of initiation of seed imbibition, and wounded using sonication. Following wounding, explants were placed in co-culture for 2-5 days at which point they were transferred to selection media to allow selection and growth of transgenic shoots. Resistant shoots were harvested in approximately 6-8 weeks and placed into selective rooting media for 2-3 weeks. Shoots producing roots were transferred to the greenhouse and potted in soil. Shoots that remained healthy on selection, but did not produce roots were transferred to non-selective rooting media for an additional two weeks. Roots from any shoots that produced roots off selection were tested for expression of the plant selectable marker before they were transferred to the greenhouse and potted in soil.

The above process can be repeated to produce multiple events of transgenic soybean plants from cells that were transformed with recombinant DNA having the genes identified in Table 1. Progeny transgenic plants and seed of the transformed plants were screened for the presence and single copy of the inserted gene, and tested for various altered or enhanced phenotypes and traits as shown in Tables 9 and 10.

### Example 3. Identification of Altered Phenotypes in Automated Greenhouse

This example illustrates screening and identification of transgenic corn plants for altered phenotypes in an automated greenhouse (AGH). The apparatus and the methods for automated phenotypic screening of plants are disclosed, for example, in U.S. Patent Publication No. 2011/0135161, which is incorporated herein by reference in its entirety.

Corn plants were tested in three screens in the AGH under different conditions including non-stress, nitrogen deficit, and water deficit stress conditions. All screens began with non-stress conditions during days 0-5 germination phase, after which the plants were grown for 22 days under the screen-specific conditions shown in Table 2.

TABLE 2
Description of the three AGH screens for corn plants

| | | | Screen |
|---|---|---|---|
| | | Germination | specific |
| | | phase | phase |
| Screen | Description | (5 days) | (22 days) |
| Non-stress | well watered | 55% VWC | 55% VWC |
| | sufficient nitrogen | water | 8 mM nitrogen |
| Water deficit | limited watered | 55% VWC | 30% VWC |
| | sufficient nitrogen | water | 8 mM nitrogen |
| Nitrogen deficit | well watered | 55% VWC | 55% VWC |
| | low nitrogen | water | 2 mM nitrogen |

Water deficit is defined as a specific Volumetric Water Content (VWC) that is lower than the VWC of a non-stressed plant. For example, a non-stressed plant might be maintained at 55% VWC, and the VWC for a water-deficit assay might be defined around 30% VWC. Data were collected using visible light and hyperspectral imaging as well as direct measurement of pot weight and amount of water and nutrient applied to individual plants on a daily basis.

Nitrogen deficit is defined (in part) as a specific mM concentration of nitrogen that is lower than the nitrogen concentration of a non-stressed plant. For example, a non-stressed plant might be maintained at 8 mM nitrogen, while the nitrogen concentration applied in a nitrogen-deficit assay might be maintained at a concentration of 2 mM.

Up to ten parameters were measured for each screen. The visible light color imaging based measurements are: biomass, canopy area, and plant height. Biomass (Bmass) is defined as the estimated shoot fresh weight (g) of the plant obtained from images acquired from multiple angles of view. Canopy Area (Cnop) is defined as leaf area as seen in a top-down image (mm²). Plant Height (PlntH) refers to the distance from the top of the pot to the highest point of the plant derived from a side image (mm). Anthocyanin score and area, chlorophyll score and concentration, and water content score are hyperspectral imaging-based parameters. Anthocyanin Score (AntS) is an estimate of anthocyanin in the leaf canopy obtained from a top-down hyperspectral image. Anthocyanin Area (AntA) is an estimate of anthocyanin in the stem obtained from a side-view hyperspectral image. Chlorophyll Score (ClrpS) and Chlorophyll Concentration (ClrpC) are both measurements of chlorophyll in the leaf canopy obtained from a top-down hyperspectral image, where Chlorophyll Score measures in relative units, and Chlorophyll Concentration is measured in parts per million (ppm) units. Water Content Score (WtrCt) is a measurement of water in the leaf canopy obtained from a top-down hyperspectral image. Water Use Efficiency (WUE) is derived from the grams of plant biomass per liter of water added. Water Applied (WtrAp) is a direct measurement of water added to a pot (pot with no hole) during the course of an experiment.

These physiological screen runs were set up so that tested transgenic lines were compared to a control line. The collected data were analyzed against the control using % delta and certain p-value cutoff. Tables 3-5 are summaries of transgenic corn plants comprising the disclosed recombinant DNA constructs with altered phenotypes under non stress, nitrogen deficit, and water deficit conditions, respectively.

The test results are represented by three numbers: the first number before letter “p” denotes number of events with an increase in the tested parameter at p≤0.1; the second number before letter “n” denotes number of events with a decrease in the tested parameter at p≤0.1; the third number before letter “t” denotes total number of transgenic events tested for a given parameter in a specific screen. The increase or decrease is measured in comparison to non-transgenic control plants. A designation of “-” indicates that it has not been tested. For example, 2p1n5t indicates that 5 transgenic plant events were screened, of which 2 events showed an increase, and 1 showed a decrease of the measured parameter.

TABLE 3
Summary of transgenic corn plants with altered phenotypes in AGH non-stress screens

| GeneID | AntS | Bmass | Cnop | ClrpS | PlntH | WtrAp | WtrCt | WUE | ClrpC | AntA |
|---|---|---|---|---|---|---|---|---|---|---|
| T5MON15 | 0p1n5t | 0p2n5t | 0p1n5t | 0p0n5t | 0p1n5t | 0p1n5t | 0p0n5t | 0p2n5t | — | — |
| T5MON02 | 0p0n5t | 1p0n5t | 0p0n5t | 1p0n5t | 1p0n5t | 0p0n5t | — | 1p0n5t | — | — |
| T5MON23 | 1p0n5t | 0p0n5t | 0p0n5t | 0p2n5t | 0p1n5t | 1p1n5t | — | 0p0n5t | — | — |
| T5MON17 | 0p0n5t | 0p1n5t | 0p4n5t | 1p0n5t | 0p0n5t | 0p0n5t | 1p1n5t | 0p1n5t | — | — |
| T5MON03 | 0p0n5t | 0p0n5t | 1p0n5t | — | 0p1n5t | 1p0n5t | — | 0p0n5t | 0p0n5t | 0p0n5t |
| T5MON05 | 1p1n10t | 0p2n10t | 0p2n10t | — | 1p3n10t | 0p2n10t | — | 0p1n10t | 2p0n10t | 0p1n10t |
| T5MON16 | 0p1n5t | 0p1n5t | 0p1n5t | — | 1p0n5t | 0p1n5t | — | 0p2n5t | 0p0n5t | — |
| T5MON26 | 0p0n5t | 1p2n5t | 0p1n5t | — | 0p1n5t | 0p0n5t | — | 1p1n5t | 0p0n5t | — |
| T5MON27 | 1p1n7t | 0p0n7t | 3p0n7t | 0p1n7t | 0p1n7t | 0p1n7t | 0p0n7t | 0p0n7t | — | — |
| T5MON06 | 0p0n8t | 1p0n8t | 1p0n8t | 0p0n8t | 0p0n8t | 0p0n8t | 0p0n8t | 0p0n8t | — | — |
| T5MON28 | 0p0n5t | 0p1n5t | 0p1n5t | — | 0p2n5t | 1p2n5t | — | 0p2n5t | 0p0n5t | — |
| T5MON19 | 0p1n5t | 0p1n5t | 0p1n5t | — | 0p2n5t | 0p1n5t | — | 0p2n5t | 0p0n5t | 0p1n5t |
| T5MON29 | 2p1n5t | 0p2n5t | 0p1n5t | — | 0p2n5t | 0p0n5t | — | 0p2n5t | 0p0n5t | 0p0n5t |
| T5MON08 | 0p1n5t | 0p1n5t | 0p0n5t | — | 0p1n5t | 0p0n5t | — | 0p1n5t | 0p1n5t | — |
| T5MON14 | 0p0n5t | 1p0n5t | 0p0n5t | — | 2p0n5t | 1p0n5t | — | 1p0n5t | 0p0n5t | — |

TABLE 4
Summary of transgenic corn plants with altered phenotypes in AGH nitrogen-deficit screens

| GeneID | AntS | Bmass | Cnop | ClrpS | PlntH | WtrAp | WtrCt | WUE | ClrpC | AntA |
|---|---|---|---|---|---|---|---|---|---|---|
| T5MON01 | — | 3p0n5t | 3p0n5t | — | 2p0n5t | 3p0n5t | — | 1p0n5t | — | 1p0n5t |
| T5MON02 | 0p0n5t | 0p0n5t | 0p0n5t | 3p0n5t | 1p0n5t | 1p2n5t | 0p0n5t | 1p0n5t | — | — |
| T5MON03 | 0p0n5t | 0p0n5t | 1p0n5t | — | 0p1n5t | 0p0n5t | — | 0p0n5t | 0p1n5t | 0p0n5t |
| T5MON04 | 0p0n10t | 0p0n10t | 0p1n10t | — | 1p1n10t | 0p2n10t | — | 0p0n10t | 0p0n10t | 1p0n10t |
| T5MON05 | 1p0n10t | 0p0n10t | 0p3n10t | — | 0p3n10t | 1p1n10t | — | 1p1n10t | 1p0n10t | 0p2n10t |
| T5MON06 | 0p1n8t | 0p1n8t | 0p1n8t | 0p1n8t | 0p5n8t | 1p1n8t | 0p1n8t | 0p3n8t | — | — |
| T5MON07 | 0p0n5t | 0p0n5t | 0p0n5t | — | 0p0n5t | 1p0n5t | — | 0p0n5t | 0p1n5t | 1p0n5t |
| T5MON08 | 0p2n5t | 4p0n5t | 1p0n5t | — | 0p0n5t | 3p0n5t | — | 4p0n5t | 2p0n5t | — |
| T5MON14 | 2p0n5t | 0p3n5t | 0p3n5t | — | 0p1n5t | 0p3n5t | — | 0p2n5t | 0p1n5t | — |
| T5MON15 | 0p1n5t | 0p0n5t | 0p2n5t | 0p0n5t | 0p4n5t | 0p3n5t | 0p0n5t | 0p0n5t | — | — |
| T5MON16 | 1p0n5t | 0p1n5t | 0p1n5t | — | 0p2n5t | 0p1n5t | — | 0p1n5t | 1p0n5t | — |
| T5MON17 | 0p1n5t | 0p3n5t | 1p1n5t | 2p0n5t | 0p4n5t | 1p1n5t | 0p3n5t | 0p4n5t | — | — |
| T5MON19 | 0p1n5t | 0p0n5t | 0p2n5t | — | 1p0n5t | 0p5n5t | — | 0p0n5t | 0p0n5t | 0p3n5t |
| T5MON23 | 0p0n5t | 4p0n5t | 2p0n5t | 0p1n5t | 2p0n5t | 4p0n5t | 1p0n5t | 3p0n5t | — | — |
| T5MON25 | 0p1n8t | 2p0n8t | 0p1n8t | — | 2p1n8t | 0p4n8t | — | 3p0n8t | 3p1n8t | 0p0n8t |
| T5MON26 | 0p0n5t | 0p2n5t | 0p3n5t | — | 0p2n5t | 0p1n5t | — | 0p2n5t | 0p0n5t | — |
| T5MON27 | 1p0n7t | 0p1n7t | 0p0n7t | 0p0n7t | 0p2n7t | 0p0n7t | 0p0n7t | 0p1n7t | — | — |
| T5MON28 | 0p0n5t | 0p2n5t | 0p0n5t | — | 0p3n5t | 0p1n5t | — | 0p1n5t | 1p0n5t | — |
| T5MON29 | 0p0n5t | 0p0n5t | 0p2n5t | — | 0p1n5t | 0p2n5t | — | 1p0n5t | 1p0n5t | 0p0n5t |

TABLE 5
Summary of transgenic corn plants with altered phenotypes in AGH water-deficit screens

| GeneID | AntS | Bmass | Cnop | ClrpS | PlntH | WtrAp | WtrCt | WUE | ClrpC | AntA |
|---|---|---|---|---|---|---|---|---|---|---|
| T5MON01 | 0p0n5t | 0p2n5t | 0p3n5t | — | 1p1n5t | 0p3n5t | — | 0p0n5t | 0p1n5t | 2p0n5t |
| T5MON02 | 0p0n5t | 1p1n5t | 0p1n5t | 0p3n5t | 0p1n5t | 1p2n5t | 0p1n5t | 0p0n5t | — | — |
| T5MON03 | 0p0n5t | 0p1n5t | 0p0n5t | — | 0p0n5t | 0p3n5t | — | 2p0n5t | 0p0n5t | 1p0n5t |
| T5MON04 | 0p1n10t | 3p1n10t | 4p0n10t | — | 1p1n10t | 1p1n10t | — | 3p0n10t | 3p0n10t | 0p0n10t |
| T5MON05 | 0p1n10t | 0p0n10t | 1p0n10t | — | 1p1n10t | 2p2n10t | — | 0p0n10t | 3p0n10t | 0p4n10t |
| T5MON06 | 0p0n8t | 0p1n8t | 0p0n8t | 0p2n8t | 0p1n8t | 0p3n8t | 0p2n8t | 0p1n8t | — | — |
| T5MON07 | 1p0n5t | 0p0n5t | 0p1n5t | — | 0p1n5t | 1p1n5t | — | 0p0n5t | 1p1n5t | 1p0n5t |
| T5MON08 | 1p0n5t | 0p0n5t | 0p0n5t | — | 0p0n5t | 0p0n5t | — | 0p0n5t | 0p0n5t | — |
| T5MON14 | 0p0n5t | 2p1n5t | 3p1n5t | — | 0p1n5t | 0p1n5t | — | 2p0n5t | 0p0n5t | — |
| T5MON15 | 0p3n5t | 2p0n5t | 3p0n5t | 1p0n5t | 2p0n5t | 3p0n5t | 0p0n5t | 2p0n5t | — | — |
| T5MON16 | 0p0n5t | 0p1n5t | 0p1n5t | — | 0p0n5t | 0p2n5t | — | 0p1n5t | 0p1n5t | — |
| T5MON17 | 0p0n5t | 0p0n5t | 0p0n5t | 1p0n5t | 0p0n5t | 1p0n5t | 0p1n5t | 0p0n5t | — | — |
| T5MON19 | 1p0n5t | 1p0n5t | 2p0n5t | — | 1p0n5t | 3p0n5t | — | 0p0n5t | 0p2n5t | 0p1n5t |
| T5MON23 | 0p0n5t | 4p0n5t | 3p0n5t | 1p0n5t | 4p0n5t | 5p0n5t | 0p0n5t | 0p0n5t | — | — |
| T5MON25 | 0p1n8t | 3p0n8t | 1p0n8t | — | 2p0n8t | 0p0n8t | — | 3p0n8t | 2p0n8t | 0p1n8t |
| T5MON26 | 0p1n5t | 0p2n5t | 0p2n5t | — | 0p1n5t | 0p4n5t | — | 0p0n5t | 0p0n5t | — |
| T5MON27 | 0p2n7t | 1p0n7t | 2p0n7t | 0p0n7t | 0p1n7t | 3p0n7t | 1p0n7t | 0p0n7t | — | — |
| T5MON28 | 1p0n5t | 2p1n5t | 2p0n5t | — | 0p1n5t | 3p0n5t | — | 0p1n5t | 1p0n5t | — |
| T5MON29 | 2p0n5t | 3p1n5t | 1p1n5t | — | 1p1n5t | 2p1n5t | — | 2p1n5t | 0p0n5t | 0p0n5t |

### Example 4. Evaluation of Transgenic Plants for Trait Characteristics

Trait assays were conducted to evaluate trait characteristics and phenotypic changes in transgenic plants as compared to non-transgenic controls. Corn and soybean plants were grown in field and greenhouse conditions. Up to 18 parameters were measured for corn in phenology, morphometrics, biomass, and yield component studies at certain plant developmental stages. For root assays, soybean plants were grown in the greenhouse in transparent nutrient medium to allow the root system to be imaged and analyzed.

Corn developmental stages are defined by the following development criteria:
Developed leaf: leaf with a visible leaf collar;
V-Stages: Number of developed leaves on a corn plant corresponds to the plant's vegetative growth stage—i.e., a V6 stage corn plant has 6 developed (fully unfolded) leaves;
R1 (Silking): Plants defined as R1 must have one or more silks extending outside the husk leaves. Determining the reproductive stage of the crop plant at R1 or later is based solely on the development of the primary ear;
R3 (Milk): Typically occurs 18-22 days after silking depending on temperature and relative maturity. Kernels are usually yellow in color and the fluid inside each kernel is milky white;
R6 (Physiological maturity): Typically occurs 55-65 days after silking (depending on temperature and relative maturity group of the germplasm being observed). Kernels have reached their maximum dry matter accumulation at this point, and kernel moisture is approximately 35%.

Soybean developmental stages are defined by criteria as following:
Fully developed trifoliate leaf node: A leaf is considered completely developed when the leaf at the node immediately above it has unrolled sufficiently so the two edges of each leaflet are no longer touching. At the terminal node on the main stem, the leaf is considered completely developed when the leaflets are flat and similar in appearance to older leaves on the plant;
VC: Cotyledons and Unifoliolates are fully expanded;
R1: Beginning of flowering—i.e., one open flower at any node on the main stem.
   Table 6 describes the trait assays. TraitRefID is the reference ID of each trait assay. Trait Name is the descriptive name of the assay. Trait Description describes what the assay measures, and how the measurement is conducted. Direction For Positive Call indicates whether an increase or decrease in the measurement quantity corresponds to a “positive” call in the assay results.

TABLE 6
Description of Trait Assays.

| | | | Direction |
|---|---|---|---|
| | | | For |
| | | | Positive |
| TraitRefID | Trait Assay Name | Description | Call |
| DOV12 | Days from | number of days from the date of planting to the | decrease |
| | Planting to V12 | date when 50% of the plants in a plot reaches V12 stage. | |
| P50DR1 | Days to 50% | number of days from the date of planting to the | decrease |
| | Pollen Shedding | date when 50% of the plants in a plot reaches | |
| | | Pollen Shed stage. | |
| S50DR1 | Days to 50% | number of days from the date of planting to the | decrease |
| | Visible Silk | date when 50% of the plants in a plot reaches | |
| | | visible Silking (R1) stage. | |
| STDIR3 | Stalk Diameter | plot average of the stalk diameter of a plant. It | increase |
| | at R3 | measures maximal “long” axis in the middle of the | |
| | | internode above first visible node. Measurement is | |
| | | taken at R3 stage. | |
| KRNR6 | Kernel Row | plot average of the number of rows of kernels on | increase |
| | Number at R6 | an ear, by counting around the circumference of | |
| | | the ear. Measurement is taken at R6 stage. | |
| KRLR6 | Kernels | (also known as rank number) the plot average of | increase |
| | per Row | the number of kernels per row longitudinally. It is | |
| | Longitudinally | calculated as the ratio of (total kernel count per ear)/ | |
| | at R6 | (kernel row number). | |
| | | Measurement is taken at R6 stage. | |
| LFTNR3 | Leaf Tip | plot average of the number of leaves per plant, by | increase |
| | Number at R3 | counting the number of leaf tips. Measurement is | |
| | | taken at R3 stage. | |
| PLTHGR | Plant Height | plot average of growth rate of a plant from V6 to | increase |
| | Growth | V12 stage. It is calculated as (Plant Height | |
| | Rate from | measured at V12-Plant Height measured at V6)/ | |
| | V6 to V12 | Days between measurements. | |
| PHTR3 | Plant Height | plot average of plant height. It measures from soil | decrease |
| | at R3 | line to base of highest collared leaf. Measurement | |
| | | is taken at R3 stage. | |
| EAR6 | Ear Area at R6 | plot average of size of area of a ear from a 2- | increase |
| | | dimentional view. The measurement is done | |
| | | through imaging of ear, including kernels and | |
| | | void. Typically 10 representative ears are | |
| | | measured per plot. Measurement is taken at R6 stage. | |
| EDR6 | Ear Diameter | plot average of the ear diameter. It measures | increase |
| | at R6 | maximal “wide” axis over the ear on the largest | |
| | | section of the ear. Measurement is taken at R6 stage. | |
| EDWR1 | Ear Dry | plot average of the ear dry weight of a plant. | increase |
| | Weight at R6 | Measurement is taken at R6 stage. | |
| ELR6 | Ear Length | plot average of the length of ear. It measures from | increase |
| | at R6 | tip of ear in a straight line to the base at the ear node. | |
| | | Measurement is taken at R6 stage. | |
| ETVR6 | Ear Tip | plot average of area percentage of void at the top | decrease |
| | Void | 30% area of a ear, from a 2-dimentional view. | |
| | Percentage | The measurement is done through imaging of ear, | |
| | at R6 | including kernels and void. Typically 10 | |
| | | representative ears are measured per plot. | |
| | | Measurement is taken at R6 stage. | |
| EVR6 | Ear Void | plot average of area percentage of void on a ear, | decrease |
| | Percentage | from a 2-dimentional view. The measurement is | |
| | at R6 | done through imaging of ear, including kernels | |
| | | and void. Typically 10 representative ears are | |
| | | measured per plot. Measurement is taken at R6 stage. | |
| KPER6 | Kernels per | plot average of the number of kernels per ear. It is | increase |
| | Ear at R6 | calculated as (total kernel weight/(Single Kernel | |
| | | Weight * total ear count), where total kernel | |
| | | weight and total ear count are measured from ear | |
| | | samples from an area between 0.19 to 10 square | |
| | | meters, and Single Kernel Weight (SKWTR6) is | |
| | | described below. Measurement is taken at R6 stage. | |
| SKWTR6 | Single Kernel | plot average of weight per kernel. It is calculated | increase |
| | Weight at R6 | as the ratio of (sample kernel weight adjusted to | |
| | | 15.5% moisture)/(sample kernel number). The | |
| | | sample kernel number ranges from 350 to 850. | |
| | | Measurement is taken at R6 stage. | |
| RBPN | Root Branch | number of root branch tip points of a plant. The | increase |
| | Point Number | measurement is done through imaging of the root | |
| | at VC or V2 | system of a plant grown in a transparent | |
| | | Gelzan(TM) gum gel nutrient medium to VC stage | |
| | | for soybean, or to V2 stage for corn. The root | |
| | | system image is skeletonized for the root length | |
| | | measurement. Up to 40 images are taken at various | |
| | | angles around the root vertical axis and | |
| | | measurement is averaged over the images. Gelzan | |
| | | is a trademark of CP Kelco U.S., Inc. | |
| RTL | Root Total | cumulative length of roots of a plant, as if the roots | increase |
| | Length at | were all lined up in a row. The measurement is | |
| | VC or V2 | done through imaging of the root system of a plant | |
| | | grown in a transparent Gelzan(TM) gum gel | |
| | | nutrient medium to VC stage for soybean, or to V2 | |
| | | stage for corn. The root system image is | |
| | | skeletonized for the root length measurement. Up | |
| | | to 40 images are taken at various angles around the | |
| | | root vertical axis and measurement is averaged over the | |
| | | images. Gelzan is a trademark of CP Kelco U.S., Inc. | |

These trait assays were set up so that the tested transgenic lines were compared to a control line. The collected data were analyzed against the control, and positives were assigned if there was a p-value of 0.2 or less. Tables 7, 8 and 9 are summaries of transgenic plants comprising the disclosed recombinant DNA constructs for corn phenology and morphometrics assays, corn yield/trait component assays, and soybean root assays, respectively.

The test results are represented by three numbers: the first number before letter “p” denotes number of tests of events with a “positive” change as defined in Table 6; the second number before letter “n” denotes number of tests of events with a “negative” change which is in the opposite direction of “positive” as defined in Table 6; the third number before letter “t” denotes total number of tests of transgenic events for a specific assay for a given gene. The “positive” or “negative” change is measured in comparison to non-transgenic control plants. A designation “-” indicates that it has not been tested. For example, 2p1n5t indicates that 5 transgenic plant events were tested, of which 2 events showed a “positive” change and 1 showed a “negative” change of the measured parameter. The assay is indicated with its TraitRefID as in Table 6. Note that two constructs of gene T5MON21 were tested in some assays, and the results are listed as T5MON21 and T5MON21x.

TABLE 7
Summary of assay results for corn phenology and morphometrics assays

| Gene_ID | P50DR1 | S50DR1 | KRNR6 | KRLR6 | LFTNR3 | DOV12 | PHTR3 | STDIR3 | PLTHGR |
|---|---|---|---|---|---|---|---|---|---|
| T5MON01 | 2p0n4t | 0p0n4t | — | — | — | 2p0n4t | — | — | 5p0n12t |
| T5MON02 | 6p0n11t | 2p0n11t | 0p1n6t | 3p0n6t | 0p0n3t | — | — | — | — |
| T5MON03 | — | — | 1p0n4t | 1p0n4t | — | — | — | — | — |
| T5MON06 | 1p0n6t | 0p0n5t | — | — | — | — | 2p0n5t | 1p0n5t | — |
| T5MON08 | 1p0n4t | 0p0n4t | 2p0n3t | 1p1n3t | — | — | — | — | — |
| T5MON16 | 0p0n8t | 4p2n8t | 0p0n4t | 0p1n4t | 0p0n4t | — | — | — | — |
| T5MON17 | — | — | 0p3n4t | 0p1n4t | — | — | — | — | — |
| T5MON19 | 2p1n4t | 3p0n4t | — | — | 4p0n4t | — | — | — | — |
| T5MON20 | 2p0n4t | 0p0n4t | 0p1n2t | 0p0n2t | 0p1n2t | — | — | — | — |
| T5MON21 | 0p0n4t | 0p0n4t | 0p1n3t | 1p0n3t | — | — | — | — | — |
| T5MON21x | 0p3n5t | 0p4n5t | 0p2n3t | 0p0n3t | — | — | — | — | — |
| T5MON23 | 2p1n7t | 1p0n6t | — | — | 0p0n1t | — | — | — | — |
| T5MON26 | 2p0n11t | 2p0n11t | 2p0n6t | 0p0n6t | 0p0n3t | — | — | — | — |
| T5MON27 | 0p0n4t | 1p0n5t | 0p0n2t | 0p0n2t | 0p0n2t | — | — | — | — |
| T5MON28 | 2p0n11t | 2p3n11t | 0p3n7t | 0p2n4t | 0p2n4t | 0p2n3t | — | — | — |
| T5MON29 | — | — | 0p0n4t | 0p0n4t | — | — | — | — | — |

TABLE 8
Summary of assay results for corn trait component assays

| GeneID | EAR6 | EDR6 | ELR6 | ETVR6 | EVR6 | KPER6 | SKWTR6 | EDWR1 |
|---|---|---|---|---|---|---|---|---|
| T5MON02 | 1p0n3t | 0p0n3t | 2p0n3t | 1p0n3t | 1p0n3t | 2p0n6t | 0p0n6t | 1p0n3t |
| T5MON03 | — | — | — | — | — | 1p0n4t | 0p1n4t | — |
| T5MON08 | 1p0n6t | 1p0n6t | 1p0n6t | 0p1n6t | 0p1n6t | 1p1n6t | 1p2n6t | — |
| T5MON16 | 2p0n3t | 0p0n3t | 2p0n3t | 0p1n3t | 0p1n3t | 0p0n7t | 2p0n7t | 4p0n7t |
| T5MON17 | — | — | — | — | — | 0p1n4t | 0p0n4t | — |
| T5MON19 | — | — | — | — | — | 0p0n4t | 0p0n4t | — |
| T5MON20 | 0p1n2t | 0p2n2t | 0p1n2t | 1p0n2t | 1p0n2t | 0p0n4t | 0p1n4t | 0p0n2t |
| T5MON21 | 1p0n3t | 1p0n3t | 1p0n3t | 0p1n3t | 0p0n3t | 1p1n3t | 1p0n3t | — |
| T5MON21x | 0p0n3t | 1p1n3t | 0p0n3t | 0p3n3t | 0p3n3t | 0p0n3t | 0p0n3t | — |
| T5MON26 | 2p0n3t | 2p0n3t | 2p0n3t | 0p0n3t | 0p0n3t | 1p0n6t | 0p1n6t | 0p0n3t |
| T5MON27 | 0p0n2t | 0p0n2t | 1p0n2t | 0p0n2t | 0p0n2t | 0p1n2t | 0p0n2t | — |
| T5MON28 | 0p2n3t | 0p1n3t | 0p2n3t | 0p0n3t | 0p0n3t | 0p5n4t | 5p0n7t | 0p1n7t |
| T5MON29 | — | — | — | — | — | 0p0n4t | 0p2n4t | — |

TABLE 9
Summary of assay results
for soybean root assays

| Gene_ID | RBPN | RTL |
|---|---|---|
| T5MON12 | 0p0n4t | 1p0n4t |
| T5MON13 | 3p0n4t | 3p0n4t |

### Example 5. Phenotypic Evaluation of Transgenic Plants in Field Trials for Increased Nitrogen use Efficiency, Increased Water use Efficiency, and Increased Yield

Corn field trials were conducted to identify genes that can improve nitrogen use efficiency (NUE) under nitrogen limiting conditions leading to increased yield performance as compared to non transgenic controls. For the Nitrogen field trial results shown in Table 10, each field was planted under nitrogen limiting condition (60 lbs/acre), and corn ear weight or yield was compared to non-transgenic control plants.

Corn field trials were conducted to identify genes that can improve water use efficiency (WUE) under water limiting conditions leading to increased yield performance as compared to non transgenic controls. Results of the water use efficiency trials conducted under managed water limiting conditions are shown in Table 10, and the corn ear weight or yield was compared to non-transgenic control plants.

Corn and soybean field trials were conducted to identify genes that can improve broad-acre yield (BAY) under standard agronomic practice. Results of the broad-acre yield trials conducted under standard agronomic practice are shown in Table 10, and the corn or soybean yield was compared to non-transgenic control plants.

Table 10 provides a list of genes that produce transgenic plants having increased nitrogen use efficiency (NUE), increased water use efficiency (WUE), and/or increased broad-acre yield (BAY) as compared to a control plant. Polynucleotide sequences in constructs with at least one event showing significant yield or ear weight increase across multiple locations at p≤0.2 are included. The genes were expressed with constitutive promoters unless noted otherwise under the “Specific Expression Pattern” column. A promoter of a specific expression pattern was chosen over a constitutive promoter, based on the understanding of the gene function, or based on the observed lack of significant yield increase when the gene was expressed with constitutive promoter. The elements of Table 10 are described as follows: “Crop” refers to the crop in trial, which is either corn or soybean; “Condition” refers to the type of field trial, which is BAY for broad acre yield trial under standard agronomic practice (SAP), WUE for water use efficiency trial, and NUE for nitrogen use efficiency trial; “Specific Expression Pattern” refers to the expected expression pattern or promoter type, instead of constitutive; “Gene ID” refers to the gene identifier as defined in Table 1; “Yield results” refers to the recombinant DNA in a construct with at least one event showing significant yield increase at p≤0.2 across locations. The first number refers to the number of tests of events with significant yield or ear weight increase, whereas the second number refers to the total number of tests of events for each recombinant DNA in the construct. Typically 4 to 8 distinct events per construct are tested.

TABLE 10
Recombinant DNA with protein-coding genes for increased nitrogen
use efficiency, increased water use efficiency and increased yield

| | | Specific | | |
|---|---|---|---|---|
| | | Expression | | Yield |
| Crop | Condition | Pattern | Gene ID | results |
| corn | BAY | | T5MON01 | 1/7 |
| corn | BAY | | T5MON04 | 3/8 |
| corn | BAY | root specific | T5MON05 | 4/28 |
| corn | BAY | root specific | T5MON06 | 7/44 |
| corn | WUE | root specific | T5MON06 | 1/6 |
| corn | BAY | seed specific | T5MON08 | 1/18 |
| corn | WUE | seed specific | T5MON08 | 3/5 |
| corn | BAY | | T5MON14 | 1/15 |
| corn | WUE | | T5MON14 | 2/6 |
| corn | BAY | root specific | T5MON15 | 2/8 |
| corn | BAY | | T5MON16 | 2/20 |
| corn | NUE | | T5MON16 | 5/11 |
| corn | BAY | | T5MON17 | 4/16 |
| corn | BAY | | T5MON19 | 8/19 |
| corn | WUE | meristem & | T5MON21 | 1/7 |
| | | cob enhanced | | |
| corn | BAY | seed preferred | T5MON23 | 3/27 |
| corn | NUE | seed preferred | T5MON23 | 1/14 |
| corn | BAY | meristem & | T5MON27 | 2/24 |
| | | endosperm preferred | | |
| corn | BAY | | T5MON28 | 1/14 |
| soybean | BAY | | T5MON09 | 3/12 |
| soybean | BAY | | T5MON10 | 3/13 |
| soybean | BAY | | T5MON11 | 6/23 |
| soybean | BAY | | T5MON18 | 2/28 |
| soybean | BAY | seed preferred | T5MON24 | 3/14 |

Table 11 provides a list of polynucleotide sequences of promoters with specific expression patterns. To convey the specific expression patterns, choices of promoters are not limited to those listed in Table 11.

TABLE 11
Promoter sequences and expression patterns

| Nucleotide | |
|---|---|
| SEQ ID NO. | Promoter Expression Pattern |
| 93 | seed preferred |
| 94 | root preferred |
| 95 | nneristenn & endosperm preferred |
| 96 | root specific |
| 97 | endosperm specific |
| 98 | endosperm specific |
| 99 | seed specific |
| 100 | seed preferred |
| 101 | nneristenn & cob enhanced |

### Example 6. Homolog Identification

This example illustrates the identification of homologs of proteins encoded by the DNA sequences identified in Table 1, which were used to provide transgenic seed and plants having enhanced agronomic traits. From the sequences of the homolog proteins, corresponding homologous DNA sequences can be identified for preparing additional transgenic seeds and plants with enhanced agronomic traits.

An “All Protein Database” was constructed of known protein sequences using a proprietary sequence database and the National Center for Biotechnology Information (NCBI) non-redundant amino acid database (nr.aa). For each organism from which a polynucleotide sequence provided herein was obtained, an “Organism Protein Database” was constructed of known protein sequences of the organism; it is a subset of the All Protein Database based on the NCBI taxonomy ID for the organism.

The All Protein Database was queried using amino acid sequences provided in Table 1 using NCBI “blastp” program with E-value cutoff of 1e−8. Up to 1000 top hits were kept, and separated by organism names For each organism other than that of the query sequence, a list was kept for hits from the query organism itself with a more significant E-value than the best hit of the organism. The list contains likely duplicated genes of the polynucleotides provided herein, and is referred to as the Core List. Another list was kept for all the hits from each organism, sorted by E-value, and referred to as the Hit List.

The Organism Protein Database was queried using polypeptide sequences provided in Table 1 using NCBI “blastp” program with E-value cutoff of 1e−4. Up to 1000 top hits were kept. A BLAST searchable database was constructed based on these hits, and is referred to as “SubDB”. SubDB is queried with each sequence in the Hit List using NCBI “blastp” program with E-value cutoff of 1e−8. The hit with the best E-value was compared with the Core List from the corresponding organism. The hit is deemed a likely ortholog if it belongs to the Core List, otherwise it is deemed not a likely ortholog and there is no further search of sequences in the Hit List for the same organism. Homologs with at least 95% identity over 95% of the length of the polypeptide sequences provided in Table 1 are reported below in Tables 12 and 13.

Table 12 provides a list of homolog genes, the elements of which are described as follows: “PEP SEQ ID NO.” identifies an amino acid sequence. “Homolog ID” refers to an alphanumeric identifier, the numeric part of which is the NCBI Genbank GI number; and “Gene Name and Description” is a common name and functional description of the gene. Table 13 describes the correspondence between the protein-coding genes in Table 1 and their homologs, and the level of protein sequence alignment between the gene and its homolog.

TABLE 12
Homologous gene information

| PEP SEQ | | |
|---|---|---|
| ID NO. | Homolog ID | Gene Name and Description |
| 59 | gi_194698766 | gi\|194698766\|gb\|ACF83467.1\| unknown [Zea mays] |
| 60 | gi_285013667 | gi\|285013667\|gb\|ADC32810.1\| pyruvate orthophosphate |
| | | dikinase [Zea mays] |
| 61 | gi_242063426 | gi\|242063426\|ref\|XP_002453002.1\| hypothetical protein |
| | | SORBIDRAFT_04g036440 [Sorghum bicolor] |
| 62 | gi_26248016 | gi\|26248016\|ref\|NP_754056.1\| glutamate dehydrogenase |
| | | [Escherichia coli CFT073] |
| 63 | gi_223975805 | gi\|223975805\|gb\|ACN32090.1\| unknown [Zea mays] |
| 64 | gi_162457981 | gi\|162457981\|ref\|NP_001105435.1\| nuclear transcription |
| | | factor Y subunit B [Zea mays] |
| 65 | gi_15802172 | gi\|15802172\|ref\|NP_288194.1\| glutamate dehydrogenase |
| | | [Escherichia coli O157:H7 EDL9331 |
| 66 | gi_146126 | gi\|146126\|gb\|AAA23868.1\| glutamate dehydrogenase |
| | | [Escherichia coli] |
| 67 | gi_193806357 | gi\|193806357\|sp\|P11155\|Pyruvate, phosphate dikinase 1 |
| | | [Zea mays] |
| 68 | gi_115840 | gi\|115840\|sp\|P25209\| Nuclear transcription factor Y subunit B |
| | | [Zea mays] |
| 69 | gi_284921680 | gi\|284921680\|emb\|CBG34752.1\| NADP-specific glutamate |
| | | dehydrogenase [Escherichia coli 042] |
| 70 | gi_188039906 | gi\|l88039906\|gb\|ACD47129.1\| EREBP/AP2 transcription |
| | | factor [Glycine max] |
| 71 | gi_162462936 | gi\|162462936\|ref\|NP_001106052.1\| transcription factor |
| | | subunit NF-YB2 [Zea mays] |
| 72 | gi_19423910 | gi\|19423910\|gb\|AAL87324.1\| unknown protein |
| | | [Arabidopsis thaliana] |
| 73 | gi_165874954 | gi\|165874954\|gb\|ABY68378.1\| At1g04370 |
| | | [Arabidopsis thaliana] |
| 74 | gi_22093879 | gi\|22093879\|dbj\|BAC07164.1\| hypothetical protein |
| | | [Oryza sativa Japonica Group] |
| 75 | gi_226508884 | gi\|226508884\|ref\|NP_001150756.\| homeobox-leucine zipper |
| | | protein ATHB-4 [Zea mays] |
| 76 | gi_226492268 | gi\|226492268\|ref\|NP_001142481.1\| hypothetical protein |
| | | LOC100274701 [Zea mays] |
| 77 | gi_15219717 | gi\|15219717\|ref\|NP_171932.1\| ATERF14 (Ethylene- |
| | | responsive element binding factor 14); DNA binding/ |
| | | transcription factor [Arabidopsis thaliana] |
| 78 | gi_4966365 | gi\|4966365\|gb\|AAD34696.1\| Eukaryotic protein kinase domain |
| | | [Arabidopsis thaliana] |
| 79 | gi_l65874952 | gi\|l 65874952\|gb\|ABY68377.1\| At1g04370 |
| | | [Arabidopsis thalianal |
| 80 | gi_21592545 | gi\|21592545\|gb\|AAM64494.1\| glycyl tRNA synthetase, |
| | | putative [Arabidopsis thaliana] |
| 81 | gi_222424295 | gi\|222424295\|dbj\|BAH20104.1\| AT3G30390 |
| | | [Arabidopsis thaliana] |
| 82 | gi_226503589 | gi\|226503589\|ref\|NP_001141333.1\| CAAT box binding |
| | | protein1 [Zea mays] |
| 83 | gi_219885341 | gi\|219885341\|gb\|ACL53045.1\| unknown [Zea mays] |
| 84 | gi_3980050 | gi\|3980050\|emb\|CAA41635.1\| glutamate dehydrogenase |
| | | (NADP+) [Chlorella sorokiniana] |
| 85 | gi_168586 | gi\|168586\|gb\|AAA33498.1\| pyruvate,orthophosphate dikinase |
| | | [Zea mays] |
| 86 | gi_162460730 | gi\|162460730\|ref\|NP_001105738.1\| pyruvate, phosphate |
| | | dikinase 1, chloroplastic precursor [Zea mays] |
| 87 | gi_21553511 | gi\|21553511\|gb\|AAM62604.1\| growth factor like protein |
| | | [Arabidopsis thaliana] |
| 88 | gi_2208903 | gi\|2208903\|dbj\|BAA20519.1\| ascorbate oxidase |
| | | [Arabidopsis thaliana] |
| 89 | gi_118546 | gi\|118546\|sp\|P28998\|NADP-specific glutamate dehydrogenase |
| | | [Chlorella sorokiniana] |
| 90 | gi_165874950 | gi\|165874950\|gb\|ABY68376.1\| At1g04370 |
| | | [Arabidopsis thaliana] |
| 91 | gi_162459403 | gi\|162459403\|ref\|NP_001105725.1\| glutamine synthetase, |
| | | chloroplastic precursor [Zea mays] |
| 92 | gi_162463830 | gi\|162463830\|ref\|NP_001105461.1\| proliferating cell nuclear |
| | | antigen [Zea mays] |

TABLE 13
Correspondence of Genes and Homologs

| | | Percent | Percent | |
|---|---|---|---|---|
| | | Gene | Homolog | Percent |
| Gene ID | Homolog ID | Coverage | Coverage | Identity |
| T5MON04 | gi_21592545 | 95 | 100 | 99 |
| T5MON05 | gi_2208903 | 96 | 100 | 99 |
| T5MON07 | gi_21553511 | 100 | 100 | 99 |
| T5MON08 | gi_222424295 | 100 | 100 | 99 |
| T5MON09 | gi_15219717 | 100 | 100 | 99 |
| T5MON09 | gi_165874950 | 98 | 100 | 99 |
| T5MON09 | gi_165874952 | 98 | 100 | 98 |
| T5MON09 | gi_165874954 | 98 | 100 | 98 |
| T5MON10 | gi_19423910 | 99 | 100 | 100 |
| T5MON13 | gi_4966365 | 98 | 99 | 99 |
| T5MON15 | gi_118546 | 100 | 86 | 99 |
| T5MON15 | gi_3980050 | 100 | 86 | 99 |
| T5MON16 | gi_146126 | 100 | 100 | 99 |
| T5MON16 | gi_26248016 | 100 | 100 | 99 |
| T5MON16 | gi_15802172 | 100 | 100 | 99 |
| T5MON16 | gi_284921680 | 100 | 100 | 99 |
| T5MON18 | gi_188039906 | 100 | 100 | 99 |
| T5MON19 | gi_22093879 | 100 | 90 | 98 |
| T5MON20 | gi_219885341 | 100 | 100 | 99 |
| T5MON20 | gi_285013667 | 100 | 100 | 99 |
| T5MON20 | gi_168586 | 98 | 100 | 99 |
| T5MON20 | gi_193806357 | 98 | 100 | 99 |
| T5MON20 | gi_162460730 | 98 | 100 | 97 |
| T5MON21 | gi_162463830 | 100 | 100 | 98 |
| T5MON21 | gi_242063426 | 100 | 100 | 98 |
| T5MON22 | gi_162463830 | 100 | 100 | 98 |
| T5MON22 | gi_242063426 | 100 | 100 | 98 |
| T5MON27 | gi_162457981 | 100 | 100 | 99 |
| T5MON27 | gi_115840 | 100 | 99 | 99 |
| T5MON27 | gi_226503589 | 100 | 100 | 96 |
| T5MON27 | gi_162462936 | 100 | 100 | 96 |
| T5MON28 | gi_226508884 | 96 | 75 | 100 |
| T5MON29 | gi_194698766 | 100 | 100 | 99 |
| T5MON29 | gi_162459403 | 100 | 100 | 99 |
| T5MON30 | gi_223975805 | 100 | 100 | 99 |
| T5MON30 | gi_226492268 | 100 | 100 | 98 |

### Example 7. Use of Site-Directed Integration to Introduce Transgenes or Modulate Expression of Endogenous Genes in Plants

As introduced above, a DNA sequence comprising a transgene(s), expression cassette(s), etc., such as one or more coding sequences of genes identified in Tables 1 and 12, or homologs thereof, may be inserted or integrated into a specific site or locus within the genome of a plant or plant cell via site-directed integration. A DNA sequence comprising one or more coding sequences of genes identified in Tables 1 and 12, or homologs thereof, may be operably linked to a promoter and/or other regulatory elements, such as a 5′ and/or 3′ untranslated region (UTR), enhancer, intron, and/or terminator region, which may each be native, non-native, synthetic and/or heterologous to the coding sequence or plant host cell. Recombinant DNA constructs and molecules of this disclosure may thus include a donor template having an insertion sequence comprising at least one transgene, expression cassette, or other DNA sequence for insertion into the genome of the plant or plant cell. Such donor template for site-directed integration may further include one or two homology arms flanking the insertion sequence to promote insertion of the insertion sequence at the desired site or locus. Any site or locus within the genome of a plant may be chosen for site-directed integration of the insertion sequence.

Several methods for site-directed integration are known in the art involving different proteins (or complexes of proteins and/or guide RNA) that cut the genomic DNA to produce a double strand break (DSB) or nick at a desired genomic site or locus. Examples of site-specific nucleases that may be used include zinc-finger nucleases, engineered or native meganucleases, TALE-endonucleases, and RNA-guided endonucleases (e.g., Cas9 or Cpf1). For methods using RNA-guided site-specific nucleases (e.g., Cas9 or Cpf1), the recombinant DNA construct(s) will also comprise a sequence encoding one or more guide RNAs to direct the nuclease to the desired site within the plant genome. The recombinant DNA molecules or constructs of this disclosure may further comprise an expression cassette(s) encoding a site-specific nuclease, a guide RNA, and/or any associated protein(s) to carry out the desired site-directed integration event.

The endogenous genomic loci of a plant or plant cell corresponding to the genes identified in Tables 1 and 12, or a homolog thereof, may be selected for site-specific insertion of a recombinant DNA molecule or sequence capable of modulating expression of the corresponding endogenous genes. As described above, the recombinant DNA molecule or sequence serves as a donor template for integration of an insertion sequence into the plant genome. The donor template may also have one or two homology arms flanking the insertion sequence to promote the targeted insertion event. Although a transgene, expression cassette, or other DNA sequence may be inserted into a desired locus or site of the plant genome via site-directed integration, a donor template may instead be used to replace, insert, or modify a 5′ untranslated region (UTR), promoter, enhancer, intron, 3′ UTR and/or terminator region of an endogenous gene, or any portion thereof, to modulate the expression level of the endogenous gene. Another method for modifying expression of an endogenous gene is by genome editing of an endogenous gene locus. For example, a targeted genome editing event may be made to disrupt or abolish a regulatory binding site for a transcriptional repressor of an endogenous gene to increase or modify expression of the endogenous gene.

For genome editing or site-specific integration of an insertion sequence of a donor template, a double-strand break (DSB) or nick is made in the selected genomic locus. The DSB or nick may be made with a site-specific nuclease, for example a zinc-finger nuclease, an engineered or native meganuclease, a TALE-endonuclease, or an RNA-guided endonuclease (for example Cas9 or Cpf1). In the presence of a donor template, the DSB or nick may be repaired by homologous recombination between the homology arms of the donor template and the plant genome, resulting in site-directed integration of the insertion sequence to make the targeted genomic modification or insertion at the site of the DSB or nick. For genes shown herein to cause or produce a desired phenotype or trait in a plant, an expression construct or transgene comprising the coding sequence of the gene may be inserted at a desired or selected site within the genome of the plant via site-directed integration as discussed above. Alternatively, the sequence of a corresponding endogenous gene may be modified via site-directed integration to augment or alter the expression level of the endogenous gene, such as by adding a promoter or intron sequence, or by modifying or replacing a 5′ UTR sequence, promoter, enhancer, intron, 3′ UTR sequence, and/or terminator region, or any portion thereof, of the endogenous gene.

Following transformation of a plant cell with the recombinant molecule(s) or construct(s), the resulting events are screened for site-directed insertion of the donor template insertion sequence or genome modification. Plants containing these confirmed events or modifications may then be tested for modulation of an endogenous gene, expression of an integrated transgene, and/or modification of yield traits or other phenotypes.

1. A recombinant DNA construct comprising:
a) a polynucleotide sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 1-20 and 22-29; or
b) a polynucleotide sequence that encodes a polypeptide having an amino acid sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 30-49 and 51-92.

2. The recombinant DNA construct of claim 1 comprising a promoter functional in a plant cell and operably linked to the polynucleotide sequence.

3. A vector or plasmid comprising the recombinant DNA construct of claim 1.

4. A plant comprising the recombinant DNA construct of claim 1.

5. The plant of claim 4, wherein the plant is a field crop.

6. The plant of claim 4, wherein the plant is a field crop plant selected from the group consisting of corn, soybean, cotton, canola, rice, barley, oat, wheat, turf grass, alfalfa, sugar beet, sunflower, quinoa and sugarcane.

7. The plant of claim 4, wherein the plant has an altered phenotype or an enhanced trait as compared to a control plant.

8. The plant of claim 7, wherein the enhanced trait is selected from the group consisting of: decreased days from planting to maturity, increased stalk size, increased number of leaves, increased plant height growth rate in vegetative stage, increased ear size, increased ear dry weight per plant, increased number of kernels per ear, increased weight per kernel, increased number of kernels per plant, decreased ear void, extended grain fill period, reduced plant height, increased number of root branches, increased total root length, increased yield, increased nitrogen use efficiency, and increased water use efficiency as compared to a control plant.

9. The plant of claim 7, wherein the altered phenotype is selected from the group consisting of plant height, biomass, canopy area, anthocyanin content, chlorophyll content, water applied, water content, and water use efficiency.

10. A propagule comprising the recombinant DNA construct of claim 1, wherein the propagule is selected from the group consisting of cells, pollen, ovule, flower, embryo, leaf, root, stem, shoot, meristem, grain and seed.

11. A method for altering a phenotype, enhancing a trait, increasing yield, increasing nitrogen use efficiency, or increasing water use efficiency in a plant comprising producing a transgenic plant comprising a recombinant DNA construct, wherein the recombinant DNA construct comprises:
a) a polynucleotide sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 1-20 and 22-29; or
b) a polynucleotide sequence that encodes a polypeptide having an amino acid sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 30-49 and 51-92.

12. The method of claim 11, wherein the recombinant DNA construct comprises a promoter functional in a plant cell and operably linked to the polynucleotide sequence.

13. The method of claim 11, wherein the plant is produced by transforming a plant cell or tissue with the recombinant DNA construct, and regenerating or developing the transgenic plant from the plant cell or tissue comprising the recombinant DNA construct.

14. The method of claim 11, comprising:
producing a progeny plant comprising the recombinant DNA construct by crossing the transgenic plant with:
a) itself;
b) a second plant from the same plant line;
c) a wild type plant; or
d) a second plant from a different plant line, to produce a seed, growing the seed to produce a progeny plant; and
selecting a progeny plant with increased yield, increased nitrogen use efficiency, or increased water use efficiency as compared to a control plant.

15. The method of claim 11, wherein the transgenic plant is produced by site-directed integration of the recombinant DNA construct into the genome of a plant cell or tissue using a donor template comprising the recombinant DNA construct, and regenerating or developing the transgenic plant from the plant cell or tissue comprising the recombinant DNA construct.

16. A plant produced by the method of claim 11.

17. A recombinant DNA molecule for use as a donor template in site-directed integration, wherein the recombinant DNA molecule comprises an insertion sequence comprising:
a) a polynucleotide sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 1-20 and 22-29; or
b) a polynucleotide sequence that encodes a polypeptide having an amino acid sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 30-49 and 51-92.

18. The recombinant DNA molecule of claim 17, wherein the insertion sequence further comprises a promoter functional in a plant cell and operably linked to the polynucleotide sequence.

19. The recombinant DNA molecule of claim 17, further comprising at least one homology arm flanking the insertion sequence.

20. The recombinant DNA molecule of claim 17, wherein the recombinant DNA molecule further comprises at least one cassette encoding site-specific nuclease, wherein the site specific nuclease is selected from the group comprising zinc-finger nuclease, an engineered or native meganuclease, a TALE-endonuclease, or an RNA-guided endonuclease.

21. The recombinant DNA molecule of claim 17, wherein the recombinant DNA molecule further comprises at least one cassette encoding one or more guide RNAs.

22. A recombinant DNA molecule for use as a donor template in site-directed integration, wherein the recombinant DNA molecule comprises an insertion sequence for modulation of expression of an endogenous gene, wherein the endogenous gene comprises:
a) a polynucleotide sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 1-20 and 22-29, or a portion thereof; or
b) a polynucleotide sequence that encodes a polypeptide having an amino acid sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 30-49 and 51-92.

23. The recombinant DNA construct of claim 22, wherein the insertion sequence comprises a promoter, an enhancer, an intron, or a terminator region.

24. The recombinant DNA construct of claim 22, wherein the recombinant DNA molecule further comprises at least one cassette encoding site-specific nuclease, wherein the site specific nuclease is selected from the group comprising zinc-finger nuclease, an engineered or native meganuclease, a TALE-endonuclease, or an RNA-guided endonuclease.

25. The recombinant DNA construct of claim 22, wherein the recombinant DNA molecule further comprises at least one cassette encoding one or more guide RNAs.

26. A method for altering a phenotype, enhancing a trait, increasing yield, increasing nitrogen use efficiency, or increasing water use efficiency in a plant comprising:
a) modifying the genome of a plant cell by:
i) identifying an endogenous gene of the plant corresponding to a gene selected from the list of genes in Tables 1 and 12, and their homologs, and
ii) modifying a sequence of the endogenous gene in the plant cell via site-directed integration to modify the expression level of the endogenous gene; and
b) regenerating or developing a plant from the plant cell.
